SEQUENCE LISTING (114 sequences)

SEQ ID NO: 1 - DNA, 900 bp, Oryza sativa
  variation (309): /cultivar=“ksh, krr, tgr, mnk, yma, tkh, akk, mtb, hit, mmm, fom, don, or ssk”/note=“ ”c“ replaced with ”t“ ”
tattcttcacgtgattcagcgaagataacactctttaaacactgcaattgccactggaagaattagcacgaatttgagatgttttttcaccggaagataagttcataactaaggtgtttcttcgtttcaacaaacaagatataaagttcaaccagattttacatttttgaaaaccttttatctttacatatatcagtggtggagttgaaatgggagatacatcaactctaaattagagaaatttttaggatacaactaaacaagtttaaccaaatttcccttgtcctaaacagcaaatgattcagtgacacattgggttgatttagcgacttcaaacctattgtcttctttttcatttttcaaatttctagctctacaactaattcaatgactactcagtttaaaacaaaacaaatggaagattggttgggagatttaagaagaaacttgccaggtggtggcttggtccgtggaggaaagagggctcaggggctaaccacctcgcaacttagggctctggcctccgtctcccgccttttcgccgagagcccgcaaggtgacagagtgcggcgaggtcgacacttcggccgttcggggtcgccgcgtcgggcgtccgggcggcgtcgtggttcgggggactgagggcagctactcagctagaccgctggagcccaaaggaatctaaggttacatgctgtcttgttgagcctattttatgggcctgcgactttgcagttagccgaggcatattggaataaatttaatttaggtctctcaatttgtcgtcgagcctgaaattcatcactggaccgcaaaactagatacatcgcattccccaacttagttaaggtagcagtggtggtgggcagccagcgaggagggccgtggtggtccgtgatg

SEQ ID NO: 2 - DNA, 960 bp, Oryza sativa
  variation (304): /cultivar=“yki, krr, tgr, ghm, mnk, yma, hez, ksh, akk, hnh, hit, mmm, fom, or ssk”/note=“ ”a“ replaced with ”t“ ”
tttaactttattgttagtattagtactagctctggttgtctatcactctgctgcctctgcacatactgatctagaacacacatgttctctacttctctgcagtcactgctactgacatgtgggccctactctctttgggccagcatgtcagtgtcagcagaggatctcattcctacagtcaatccatgtgtgctactccgttaaaaaaacgaattccaagctacaaacctaaacacgtttttttggacggaggggtatatataaacaaagaaaaagcactgtaggtacataatatagtactagatcagtcttattacagctgttcaaaaacagttcagtatatagtgaatctagttggtctgttgctactgcagttaattggctctggttgcttttgttgatctgttgctactgcagttaattagctccggttgcttagttgatcaagttaattagctctggctgtgccctaatcaaaattcatatatagtagcttcaagcacgacataccacctttcctaccttctggtggatactcctctcttttataatttctgcagtaagcttgaaacataagtagacactgccattaattaaacaagcacagtgaattaacccagatatgtgtaatctgcatactaattaaattaggttcgtgccagttcaaggcagccacaaccacatacaggcgatccatatattgatttatatatctgatccgtttgttgaggttggtgcatcaatcccccctgaagcagctatgtcgagcctaattgcgatttgattaatcaatttttctcatccaacgatttaattatgcgtgattttaatgattcgatcggtacagttttttttctctttcttcagtgctagtgcttctactagtattcgtgacaataacctgtcggatttggaatatatgattgctgatgtggtgtcgtttttattaacaagcccttg

SEQ ID NO: 3 - DNA, 840 bp, Oryza sativa
  variation (201): /cultivar=“ksh, hts, mth, yki, krr, tgr, ghm, mnk, yma, hez, akk, ash, ank, mtb, hnh, ymt, hit, mmm, fom, don, knh, or ssk”/note=“ ”a“ replaced with ”t“ ”
tagcattaggataaaaggggcgctccttcaaaactttaaaatatcaaagaacacccttttgagattgaattgcttcttctggtctttgcctcttctttcgcttttcagcaccagaatcgtacttcctatttctagctgacataatgaggattgaggaaataagtgtcttcctatttcataaacaaaagaaaagtaatttgagtcaaacagtcacatcactatttaagttttgattcaatcgatagtttgattcatattacatctactatttgatacgagactcaatgtctcaactcttaagtctaaaagtaactttccaatgctgcacaaaggtagtagtcagggacacgaagataagtggatgagaggcactgacaaaggtagccggccaaccgcttggcattgatggcgcttgcccgttggtcgctcgctgcctcgtgttgggctggggtcgggactttgcaggcatcgtcatttcatcgtcgaatttgaaatcgagattgactccagtcacacgacatgactacacaacagtgtgacttgatctcgttcgcctctcagcctccaatgcacctgatggcagatgggcctctctaatcgattcacaggtagaagcaggattgtggctcggctatgcattaatgtgcgcctctccgattaacttgggtgccccaaaaaaattgggggacactctatcatcgccaatgtcgcacacaaccttcgacaggcttgcccattagtgtgacactcctgcccacatcactgctccattgtcatccatcaccttgtcgaccattgtggtgtgccaaaccgcggctgtcgtctgtttgtgattttgta

SEQ ID NO: 4 - DNA, 960 bp, Oryza sativa
  variation (377): /cultivar=“ksh, ymt, don, or knh”/note=“ ”t“ replaced with ”c“ ”
tccaaaatccacatgcaatgtgccattccataggaatttcatgggatttgaaaatcgtcaatcctttgaatcaaatggccaaataggaaaatttcgtataggatttgaatcctatgaaaatcctatataaatcctttgattcaaagggccctaagtttcgtacgtgtgcaactgtgcatccagcacgtactactacgtactcctatgtacttgtagtggtgtagcctatacatatgcatgaagcgttccaggaaaaataggagtctcagtaatttgtgcaggcatgcggcccatggagtaatagaccatgctgaataatttcagttcaaatttcatactccaactgtaataccatacgcaaaccatcaacttacaatactgatatacttgacatttcaaaataacatagcctttggttttagctgacgtagcgactgagtaagctagcacgaggctcatatgggtcccacatgtcagcggcccaatcttcttccacatctctcctctcttcccaatcctaatctctctccatgcctctagggtggtgggggtgggaagaagctcgacagcggtggccaacacatacgcaaggagaagctcgaggactgcaagacgactttcttttcgcctaccactggaaggcaacaccttgtttccctgccttctagttgagcgaggacactgaatgcatggaggtgttgtgacccaaatctacggcagaatccctcgccggaagttcgccggagatctagcagagaagaggcgagaagaacagggtagaaaggggaaacacgaggaagcagctggggaggaggatatttttcatttctttcatgaattgttttctcaatacagctggagtatatatactcacgcactccacccctcttgcccttagggcccaccttgtcatagacacttctatctataaagagtagaagatgtattcacttctgaaaa

SEQ ID NO: 5 - DNA, 840 bp, Oryza sativa
  variation (163): /cultivar=“hts, mth, yki, krr, yma, hez, ank, mmm, fom, or don”/note=“ ”t“ replaced with ”c“ ”
tgctgcatgtttcagtccaagctaggcgccacagaacggacaaaagtaagaaaatcgctacgtacaactcacgtctgaccagcacttagctgctaattgccctagccacatggagagaagttggtctgcgtgaagcgtaacgattggcagataaagttggattggatgggaatcggacgagggagtcgaacgtatagcaacactccagaagtaaagcagtaaaccgaaaaagttttgctatcacttgtatgaccgtctccacaagtggcgactggcacgacatggccactcgacagaaccgcacaacaaatgctgatcctttgcccctattccatgcgaagttgcgacttgcgagtctttgggcagggcatgcactactgacaagatgaaagaagaaaatcaaccgtaattcgggcgtgcactgctgcagaatagtccttgtgatcatgtccatgtgaccatgttcgttacgttctgaggcgtcgatagcgagcgatgctggtaatcgtgaccaatctcgttcacgtccaccttgttgacgcgcacgtacgtcgctatatatgacaacgtcctgctacatatagccttgctcactttcggactttgacgtatgtgaagagagcacgactaggagccactaatcatatggtttggtacatgagaggatatgcatgtttcactttgcacccaacatgtactgtactcatctagtcatcctactagtattttccatcggtgtccctttctcctgtgatctctcgctttgcacacaaacctcgcaacaaaacggtctcgctgtcgcctttcagcgcttcggcaaaggattggtcggttcatgag

SEQ ID NO: 6 - DNA, 900 bp, Oryza sativa
  variation (515): /cultivar=“mth, krr, tgr, ghm, mnk, yma, ksh, akk, ank, hnh, ymt, hit, mmm, fom, don, or knh”/note=“ ”a“ replaced with ”g“ ”
aattattattattattatttccaatcagccatatatatggcttgccaaccgatggcagcacaaatctttcgcgtccgtttgaacccattcttcaaacttgaagttggatttggacgtaatagaaggtgcagttgttcacttgttcctggcatcaacgtgtacggttgaacaaatcggtggatctcttatggttaacgtgccctgttgatgtctgaaaacccatctgttctttgttctaactcctatcttatcctctcattttttttcgcttggtctcaacttcgtgttctactagttttgaacgagtcactcactcggactcgagagctctgaacttctgaacaagccaaaatgctgtctgaaccgagatcttcttggcgctgtcagcctgtcacaaactcgcaatccaattgcacttccagcggttgagcaggttcaattcaacatgactttcatcaggagatggtaagttaggaacagattactgtcacaactcacaacagttattactactatcgcaacaaatgctagctgtccttatcctcatcgactggatacttcagaaacaagcataacagtagcattggagcaaaggacacagcatggctagaagtagatgctgctgcctagagatatcatctcgaattcatggcatgaacaaacgtcgttcatgcagccatgcaggaataataagctcagaacaggattcaggacaaattcaagctatctacaagcttgccagcatcatcatattataataattgctttaatagtcagcaaactcgtacagaatagccagatccaaatttccacaaactatatatcatcatcaggaattttaaaaagagaactcggaatcgatttcgcatgatattcgaggacacccaagccaaactgacg

SEQ ID NO: 7 - DNA, 900 bp, Oryza sativa
  variation (247): /cultivar=“hts, mth, yki, krr, mnk, hez, ksh, akk, ank, hnh, ymt, hit, mmm, fom, or knh”/note=“ ”a“ replaced with ”c“ ”
taaccacttgcttcagttgctgcatgcttagtacatcagtactgtcatgccattggttggtgtggctgtgagtgaacattgtgcagcagagaagcaagcaacaatagcattggacccccaagaaccagtacattatctctatctgtgacagagaacacaagaatgcaaatgctgataaagaatcaagaaagcattgtgcaagcagcaaggtgagtagagagtgatggaagcagagagaagctgcagactagtgatgaaaatgattggtgagtacagtgtaacaactaacaacaagtctctatgaagaagcaggtactaagcatgcatgtgtgtgtgtgtgtgatggcatgtggtatcaatgcttctggggttgttcacttgtccaccagagcaaccaggacaagtcttctcactctaccattccggtgtcattttctctctcaacccctcctcttgttgctttagcaagcctgcagcttaaactagattatgttttctttcctcaataaagattaatagtattgttaatcatgacatctttccctcacctgtttctctctcaagagagaggaggaggtgcacaggcacagacagctcacacaaacattgtgttgttcatgtctctttcttgcctacctttgttgaactggtttgccttgggagacacacaggacactcgaggctgcctggctggcctctttgtcagggagaaacctgctaatctgctataatagtgttgcttataattctatgattctatccatcacaaaggacacagtatagctgcatcctttaactgcagcttgcaggcctttttcatcgtttacttgttagcttatcagccgcgaccaaaatttttagtactaaaactcaatattagagttgatgttagggtttttt

SEQ ID NO: 8 - DNA, 900 bp, Oryza sativa
  variation (247): /cultivar=“mth, yki, krr, tgr, ghm, mnk, yma, hez, ksh, tkh, akk, ash, ank, mtb, hnh, ymt, hit, mmm, fom, don, knh, or ssk”/note=“ ”a“ replaced with ”t“ ”
tgtatagtgtcaaatttactcataggttgtttgtttttgcgatggagggagtattggtttgactggatgggtcatggaaaactggaaaagcaacagcggtatgcatggcaaaagagggacaaaagaacaagacgaaacataggtaggattgcaggatgctcaagtgagaacttgtagttgtagatgaagtgaagtgacaagccgaagtcccgtgaacgaagcaacaaaaaattgtgggagttttccatttgttgtatgtgtattatttgcgatttgaaatccaggctgtgtttagttccttccaaagttagaagtttgggttgaaattgataccatgtgactgaaaagttgtgtgtgtatgacaggttgatgtgatggaaaaagtttgaagtttgaattcaaagtttggatctaaacacagccccaatgtttaaagagaactttaacgattaaatttggccacgaccggtaagccgataaacaaaagatgagaataaagtactgtatatacaacttccagcctcatcttttcacttatgcttatgtttatcaactaaaatttaaattttcaaccttaaatttagagttgattttagggttttttttatcgaagtttattttttagcctttacttttagatcgtaggaacacgtatatgaaaaaattatttttcatttgcaattataccgtttgtcttattccctatataagcgaaacgagggaccttccctgtcttgcttgtgatcatcagtcatctcatctatccgctggatgtgaagttacgacagaaatgatccatcgttcaacttgaattacacttgtactactagcggtgtacgctcgcatgtcagcgtaacgaaacgatgacatcgccatcacagtaggagtattggtactaat

SEQ ID NO: 9 - DNA, 900 bp, Oryza sativa
  variation (475): /cultivar=“yki, yma, or ssk”/note=“ ”t“ replaced with ”g“ ”
attacaaaaattatatgaacatgcttaaacaatttacaagaataattctggttgatggcttgaggcacgtcaaaatgtttcataaagtgtgaagtgagtgtaactcaactggttatgtttttttatggaatcagtctacctaagttgaagtcctagacttacgccgatgcttgtatttattgttaatttttttttcgtgctagacgcctatcatgacttcgttaatctcaagatatgctcgcacagtctttcggaggtgctcatatgagtaagatgtgcgtgtgtacgttcatatgagtgagtatacgtgtgctacgagagtctgcgtatacagtgtgcttctaccaaaaaatgtttcagagtaaatttcacaaaactgcaggtactttgatcaaattattataaaactacagatttaatgtgatgtattacaaaactacatatttaaccatgaaattattacagaactacagatttaagattaagtatcacaaaactacaaatttaataataaaattatcacaaagatataggttttggggtttaaattcttagcactaatatgttatgattgagttataaatatctaagttttgtaattaaattgatgctaaacatatagttccacgataattttgttactaaatctgtagttttataatattctaccttaaatctataattttatgagaaattcagtgttaaatctgtagttttgtaatatattatcttaaatatatagttttgtgaaatttaatcaatgtttcacaggagacgtggcatatatgtatactccacagagcgtgtaattaaacgtaattaaaatatgaccaacatgaacgacggaagactacgtgtgaaccagccagctaattggccctggaatccgtgatgaccaag

SEQ ID NO: 10 - DNA, 1020 bp, Oryza sativa
  variation (209): /cultivar=“krr”/note=“ ”g“ replaced with ”t“ ”
aaaagtcccacggaaacagccaaaagtttattaaaccttaccattgaatgcagcattggtgaccttgctgcccttgaaagcttagttagttcattggtatcaaaaggagaaatttcatccaacacggtgcactcttgtttcccttcaaagtttgctcatagcaacctttcaaatgctattttttacagtttagttacagtaatgctaagtactccccccattccaaaatatagggcacaacgattttttcccctaatgttgcataatacgaggttcgcatgcatgcgtgcatgctattgactagcacctccccctcctctaagttctatttttaaagcctctaccctcaagatctctgatctctaatcccattgggtgcatgcattttatttattgggatgatccaaattagaaggtgataataattttttcttggtttttgcgtaagagatagttgctcattatattttggaatgtaggggagtactcatttattctagcacaccaatctcctgtgcaccaaaagtgattctgcacatagattgagaatgcaaggtagtactaacttgcaattaagtgagtgcattaattgctgaatatgcataaattaagaacttaagatgcatgcaaagaatattgctcccagtttctccactttctgatgtgaacttcccttatctagatcctacagtgggaacttttttctgttcatcttgaagtatcttttgttagctgctcatcaaaataatttatattgcctataacataacttataccattttgtcgaatgttatttatctaacttcagtgacacctattatcttttgttggggaagttcacacttgttaaatcccattgtcttttgcagataacagccctgtgggattatttttgctttcacatcaatggtgtgaaaccagtgcaaagccgtggagctttatcgattctttgcatggcagcaaagtcatctcccagcattttgggtactcatttgcaagatattattgatattgggtttg

SEQ ID NO: 11 - DNA, 900 bp, Oryza sativa
  variation (612)..(613): /cultivar=“krr, tgr, yma, ksh, tkh, akk, ash, mtb, ymt, don, or knh”/note=“ ”ca“ replaced with ”ag“ ”
cttaatcccttaatcttcatgtttagaagattcgaacgggggatccagtcttcaacttggcacgcacacgtctcaatctgcctctccattaatagccaaacaagctgtgtggcttttctcttgcaacttgcagctgtgctgatgttgctgcattctggtgaactaggctaaaaggcattttgtggtcaggccctgtttagttttacggtgaaaagttttggcgtgtcacatcggatatacggacacacatttaaatattaaatatagtctaataacaaaataaattacatattccgtctgtaaattgcgagacgaatttattaagcctaattaatacttttatcaaatcatggcgcaattaggcttaaaagattcgtcttacaatttacacgcaatctgtgtaattagtttttttatttatatttaatactaaatacatgtgtccaaatattcgatgtgacatgatgaaaagtttttgcgtgaaaactaaacaggacctcatccacattgccatggatacatatcattcatgccatggctagctacctcttgataatagtagaattgtcacgccccgaactagtcccgaccggaactagcccgtgacgctccaatttaacctgttaatcgataccagtcccaggaaatagtgctggtatgacagggagacagaatatcacagcaacagaggtctctttattatagagtagaggtacagtcatgttgggctgcggacagatcccgagctcacaactgcattacagaagggaaacggaagccaggacttggaccaaacaacacaggcgcgacttgggaactaggccgaaaccctaaaactcatcatagccggcttgctcctggaagaactcctcatcagcaggatccgcttcatcttcttca

SEQ ID NO: 12 - DNA, 960 bp, Oryza sativa
  variation (765): /cultivar=“ghm, ksh, ymt, hit, mmm, fom, don, or knh”/note=“ ”g“ replaced with ”t“ ”
acggaaaatgatgtaatcttggaccactctctgtgacctgtgttatgacttatgactgtgctgcaaaggggagtatgaattattgttctcaaaactagagatcactcatgctccaggaagccttgaatttgtcttgatttatactgaaagtaacctggattcataaaattcttgtgttcgaagcgaatggttgaggaatattatcgtttcattgagagagagatttcatctcagctagaaaagttaatacataaaaaaatgttgctagatacctcattgaagacagtttaacaccaatggaaaaaaaatgttgcaacatatacctcattttatttcaacattgcagtattaaaagaaatcttttatatatgctccttttaaaaaagcatcaagatgtacaagtttttaggggtttaacttggtcaggaagaggatgtgcatcattgtcaggaagacaacggtgtgaaacctgtcatgaatggtagcctcccgagacttgagctagaggacctagtaacaccgaggcatcaactagccagggatgcaagtaggcaatcaatcgaccatctctatgagagcacgcgtgctaatttagtttaacgagtttcagatactacctatgtcctaaaataagttaatttttcatccatcacacatataccaatacaaacatcaaaagattagaataccagtcactagatgaatagaagtcggggtactcaaaatcgttcatatgcttgacaagagagtcgagatgatccaaagtaaaacaaatacaagattattcgattcagattgaaaacattggttaaaagatagttcaaagcaaaacatcggtaataaaagatgatttaaagtgaaatttgctcattattatgataatagctcgtttgatttacgaggttggcgactaattaaggcttatttagttcccaaaataaaaaatttcacgcagtcata

SEQ ID NO: 13 - DNA, 668 bp, Oryza sativa
  variation (571): /cultivar=“mth, krr, tgr, ghm, mnk, yma, tkh, akk, mtb, hnh, or don”/note=“ ”g“ replaced with ”t“ ”
gaagcatcttaattccagacaagtcaaatttcatagcaagggacgatgttatgattatttattgaattgtacagtactattcaacctgacaaacattgtgcaatcacatggaaatggagcgttcatttatcaaatttgcacgaattcggcgatctcaacctcaagaggagcaactaccgtattcatcctcaatgttaatttctccccgaacatattatcctactaccgtattcatcctcaatgttaatttctccccgaacatattatcctttcgtgcttgatctaattttaggcatagctcaaaattagtgcaactaatctacaaactgtgaatggacaaaaatatacagcttcagcttctcaaaaccacttcccccattcgaacctgaacaaaacccaactctgatggcacagtaaataactaactagggcaagaaccatcgcgcgacacgggcgcgggctagatcgatcgatcggtcgatcaagcccctcccccaagagggaaacacgaccagcgacagcgatccatccaacgccgtcgcatcattcacagctatagcctagcttggaggaatcaaaccatggattttggccttgacgttgtcgatgtgtcgctgctctccacctcgagaaacntggccccgtcaggtcttaaatacgggtgtctt

SEQ ID NO: 14 - DNA, 900 bp, Oryza sativa
  variation (660): /cultivar=“hts, mth, tgr, yma, akk, or ank”/note=“ ”a“ replaced with ”g“ ”
ttataccacaggtgctgacattaatatgcttttacttcagtttgtgttttgttctctgtttaatcctgcatatgcctgttaaatttattacaaagactatattaaactagttttacctggcgaaaatattaaactagctttgatagttcttgttgcaacaacagaaccgtatttgttttatttcaaatattatgttccattagcggaaagagcttggttgttttgttacctcttttttttggcaaatgaagaatgctatatacaagctagattgcaatcgtatatcaggaaattgactgatcatgtatgtcgacatgtcgtcttttatgggagatgaagttttaacttcccccataactctgtttaggctaaatgtagttttgcagaaattttctgcctaaatctattttgtactttgttgatctaacattccttacacttagtttctccatttattgattgattatttttctctgtttgttgaggccttagcatgttttgcttcctccttttgctggcaggtgctgcgtgaggttaccagtgatactattggagcttgtatatgatgtgtcctttgggaccattcttctgcatagctgtgcagaagctgctactagtttgttggagaacctgttggaagacagcttctgcttgtttgtataataagatcagcttctagttagtattacttataagttgctgcagaattttgtcgtttggcagcaccgcagaattttttactgtgtagaagctgtagaacatctatatatcacttttcaatttgaagaattgtaaagagaggcaatggccgcattctaagcaggtgctctatggaaaatccctagttgcgcatgtcatatagttagccatactagtatatagtagtatgttggtaataaa

SEQ ID NO: 15 - DNA, 490 bp, Oryza sativa
  variation (223): /cultivar=“ksh, hts, mth, yki, krr, tgr, ghm, mnk, yma, hez, tkh, akk, ash, ank, mtb, hnh, ymt, hit, mmm, fom, don, knh, or ssk”/note=“ ”g“ replaced with ”a“ ”
ccttgtggtcacacttgcggcggttgcgagggcggcccgcccagaagaaaccaggccgggcttggcccgccgcgggtcagcatcctcaccgacgactcacctgcacttctacttccacgacaaggtgagcaagccatcaccgacggcagtgcgggtggtggacccggtggacccgtcatcgcggtccttctttgggatgatcaacgtcatggacgatccgctgacggaggggcccgagcccgagtccaagcccatgggccgggcccaggggctgtacatgggctcagaccaggccaagctgggcttcctccaggcaatgaacctggtgttcaccgacggcacctacaacggcagcgtggtcaccgtgctcggccgcaactgccccttcgacgacgtccgggagatgccggtgatcggcggcaccggcgccttccgcttcgcccgcggctacgcccaggccaggacgcacaccctggacctcaagaccgga

SEQ ID NO: 16 - DNA, 460 bp, Oryza sativa
  variation (55): /cultivar=“krr, ghm, ksh, tkh, ymt, hit, mmm, fom, don, or knh”/note=“ ”t“ replaced with ”g“ ”
tggcatctttgcatgttgagctttaagatgtagtgggctttaactttatagaaatataggattaattcctatagaatgtcatgatgcaggatgtcattaataatcctccaagctgttcccttttaacttttttccctgttacttgaaacttgactaaggattctcttcgtattaatgtggattgtgtcactgaccatatggttgtatctttctttcagcgcttcgctgggacttggaatgtttgttgtttttcagtgctttcatggccatggaactcagaatgtctccaacgtgcaaattcttggttgtgatctagaagatggttatttgtttgaaacaatggaagcacttgatgttcccttagcatatacacttgtgagcttgtgttgatagaattgtaaagcttacatatgttttagttctactattattttgaagagggaaatgtgcagctggatgc

SEQ ID NO: 17 - DNA, 314 bp, Oryza sativa
  variation (163): /cultivar=“ksh, tgr, mnk, yma, hez, akk, hnh, ymt, hit, mmm, fom, don, or knh”/note=“ ”g“ replaced with ”a“ ”
ttgaatcggttgcaggagagggcggtggcgatggcggagttggttgggccgcgggtgtacagctgctgctgctgccattgccggaaccacgtctgcactccacgacgacatcatctccaaggcctttcaggtgaagaagaacttgagttcttggggatttgtggggctgattgctcaagtgacaaatactaatcttaggtcatgtactgacaatctagattgaattggatttaatcactaggcttctgatgtgcgtagtgccggattgatttggtatattatgctaaagaaggtaaaaacatggcatagccgca

SEQ ID NO: 18 - DNA, 644 bp, Oryza sativa
  variation (421): /cultivar=“ksh, krr, tgr, mnk, yma, hez, tkh, akk, ash, ank, mtb, hnh, hit, mmm, fom, or ssk”/note=“ ”a“ replaced with ”c“ ”
cgaccccatgaagcttttgcctctctcacgcttcttgccacagccaaagtatgatgctagcctaatttatagcttactgtttccggtgttaaatttgcttgtagattcgggttcacgtgcaaacttgaattgataacaccatgtcatgccaactgctatctttctcccaacaagtatttctaaaactcaattgaacattgataattctcaagaaagctaataagtgttacaaatactagcagctctaagaaatatattcaaattctaatgtatgcctattaagcccaaagattccactattgtagtctgcattgtttggaattaattgatgaatctactgcaggttctgactacagaaatagtgcagcttctctgtcctatatgactatacgaaatgttacaagcaaagcatgaggaatgaatataaaaaactaaacaaatagtgaaatatctatctaattaacaccaaggagttgcgtaactctgtttgccttctctgcaggggccaaagtcaaggagggggcagaggtggtggccgtggtggtggaagaggcggtttccgtggccgtggtggtggtggcttccgtggaagaggtgcgccaaggggccgtggtggacctcctaggggtggaggtcgtggattt

SEQ ID NO: 19 - DNA, 549 bp, Oryza sativa
  misc_feature (3): n= a, t, g, or c
atntccccctatcggatcggtcatggagatgctactgccaccaccgatgaactcctctcccagtcgcggcgaatcaagcgaccccgatgaaaaatcgagctccccggcgacggatcgacctgccacgatggcggattgagcggctcacctctcctcaccggatccagccagtgtcgtggccatcttcgagctcgagctgcatgcctccgtgcgacagcggcggtggatcggacaagggtgacgcggatctgtcggcctccaccccagatgagcgattttccactctaccggattgagtgtatatttggctttgtcttttatctgactggatttctcttctttttcttcttaattaggattcaattgttcttaccataaagatgtttttaggcccgatttggttaggttttgggggaatttgggttaaactctatcggttttctataggagagacggggatagattcggtcggtttctttaggagggacggacagaggaagtgcggagggcggaggggatcgtgaacaaaggggacgctccaccaaaata

SEQ ID NO: 20 - DNA, 900 bp, Oryza sativa
  variation (141): /cultivar=“yki, krr, or ghm”/note=“ ”a“ replaced with ”g“ ”
gctagcttggccagcagtacgtgagtctgacgatgcatgcatggatgaccctgctaattaattatacttcctccatactcatagaaaaagtcctttagaacaatatttaaatcaaacatttaaaatataaatcatgaataactcttaaattgttgagtttaaaaatgtaaaaattatatgaatagatttgtcttgaaaaatactttcataaaagtgcacatatattacttttcaataaatatttttatagaaaaaagaagtcaaaattgtgttttgtaaaccgtgtcgctgtccaaaacgacttcctttacgagtatcaaccaatcgaattgccctccctctcaaaagtcaacctcctccaaattaaaggcatgcaagacgccaaaggcggcagatctgtattcttcccgtggacggtgtgcgcatgcatgcgtacaaacttttttttttgttggatttggtacgagcgtagctgataaagatagctagctcatcagcttccttcacagaatcacaagaactagtggcatatgaatcctacatacttctatccaattcgatcgatcattcaccttgtgcctatgcaataggcaatatctgagctagcgaacacagtaactctcccctcccccctcctcacgcgccgatcataaattaatactccctccgtaatgtatgacgccgttaactttttaagatgcgtttgatcgttcatcttatataaaaaaatatataatttttattatttatttaaagtatgatttaactattatatttgtatttgcacaaaaatttgaataagataaatagtcaaacatcgatcaaaaagtcaacggcatcatacattaaaaaataaaggtagtattcaatattttgtaaaatattgatcggacttgtaaa

SEQ ID NO: 21 - DNA, 960 bp, Oryza sativa
  variation (480): /cultivar=“mth, ghm, hez, or don”/note=“ ”t“ replaced with ”c“ ”
aacagtccttgtatgcactgaacgtactggtgcggcttgcttgggcaatcgtcaacggtcaagacgtactaaagagtggattaacaaaatgaatgttttactaactgtatagtgaacacaagcgggcacctatagtcgtaacgaccgccgccaaattgcccagttgcgtacgcgagaatcgatcgatcgagccgatccgatcagctagaatattcgaacggaataaagagaacatctccatgtcctgatacgtgtgtacacacacgtacgtacgtgtatacgtatacgcgcgtgtgtatgtgtacatatgtatatatatatatatatatacatatacatatgtatgtatgtatatatatatatacatatgtatgtatgtatgtatatatatatacatatgtatgtatgtatgtatatatatatatacatatgtatgtgacggagctcgtggctcctcaccgggagaccgcgcaggcccctctttgccggttcggccgggggcttagggtgagatctcaagctctctctctctgtgtgtggaaagatcgtctgctagcaagaaacgcgagacaccggcgatgtatacaggttcgggccgctgagaagcgtaataccctactcctgtgttttggtggatctgtgtatgaatgagctacaaagtgtgagcccacctctcccccgttctaagctctgaatctggcaagaatcaaccaaccccttctctatgggcaaggtcctccttttatacttcaaggggataccacatgcacccttccctttccaaactggacttttcttctctttatgaacggagattggtatggttgccgtccgaatgacacttcgatgggacagcccacacctacctccactcccggcggagacgggcgcaacgtgggatcgtggctgcccgttgctgacgcgaccagtgtcagaccggtcattcttgtccaccacgcg

SEQ ID NO: 22 - DNA, 900 bp, Oryza sativa
  variation (175): /cultivar=“krr, tgr, ghm, mnk, yma, ksh, akk, ymt, hit, mmm, fom, don, or knh”/note=“ ”g“ replaced with ”a“ ”
gatgacttctactccctccgtcccctaatataagggattttgacattttgctttccttgtttgaccactcgttttatattttttgtaaatataaaaaataaaaagttgtgcttaaagtactctggataataaagtaagtcacaaataaaataaataataatttcaaaattttctgaataagacgaatggtcaaacagtgcaagtaaaatgtcaaaatcccttatattaagggacggagggagtatgtattacctccaaaatatagtaactttaagacgagattagataccacgaaaatatattcttaactctatgtattaggttgttatatttttttaacagagagtagtatcaattcaaagtggattaattactctttccgtcttaaagtataataacttctaagattcaaaatttatccccaaacaaacaacttttcacctacatttcattctcaatcgactacaatcttccactccatatattttattttctctaccaatcacattctttttcatttaacttcacactctctcttaaaacttttatattttgatacggaggtagtacaaaatttctgatcggttgatgtgcagtggcaaaagagctcatgagacattccactgcacagacaatctgggccttcttacatggactggcacaatttaacatattccaagctgtgcctggtggatgtagggatgcaagtaggtcaacccgtaaatccacttatatgcaaaataagtagatatttacgggtttaccttactaatttggtttataaatgggtttatgggtcgacccatttgcatctctaggtgcgataagtcaagccacgacatgaaaatgggttaccacttatttgacgtataagtagattggcgggtccgtttaattgca

SEQ ID NO: 23 - DNA, 960 bp, Oryza sativa
  variation (131): /cultivar=“mth, tgr, mnk, tkh, ash, mtb, hnh, hit, mmm, don, or ssk”/note=“ ”g“ replaced with ”c“ ”
aatctgatatttcttctggttcaaatgataattgacacaagtgtgccattttgcaaaaaccatacctttaatttcattttatggtacgtatgccaagtaaaacttgtgaactacactatttagttgcttagtcataaagaactcaagtattctttttttgaggaaaagccttagaagaggacagggagagcctgttttcattaaagaagaagagacttggcccagtttttgaggggaaaccaggcccaaaaacctcagaactcaagtattctattatatgaaacttaataaactgcgtcaaagctgtggtcttcttttctgttgttgcacatcgcaagtttaagcctgaaatatgttatttttccatgttgcccatttctcaataatggaagctttattaaaactcagtcaaatacaacaagatgatacattctaattgagcccactcccgacctctgcaagaaatgcacacagccacaaaacatgacctatctagacccccattgccttcttcatgctcttaatttctgtaacatactgttcacctgtctggctatctgggatttttcaaggtgtacacctacggtcattttgatggaaggtgaaggggaattacctcaactgaacagaggatgctggttatacagaactactaacatcgtaagaacattctatgattctgatgcagacatacagtacagttttaatctaatcaaggaggacgccatatgtgggacaccgcagcgaagtgtaaggagtcctagaacatctaccttaggagttaagaagaatctatgagattgtatgtataaacatcaggtttctgcaaatactcttatctaaattcctaatgcctgtattgcaaatacatactttcagttcgcattaaagtgtgatagtatgtatgtcacctttgttctcaggtagcttggcaaaatggcaggtcagacggatcaagct

SEQ ID NO: 24 - DNA, 723 bp, Oryza sativa
  variation (236): /cultivar=“krr, ghm, ksh, tkh, ymt, hit, mmm, fom, don, or knh”/note=“ ”a“ replaced with ”g“ ”
gagatatattgcaatattttgagaattatgtgaaatgatgatttaacgtgcttggatttttgtaagctctaaaattttaagtgaggataaactatataagcatataggatattataaaaagtgaaggagaggtatattgaaatattatgtggattatgtgggatgataatttaattaacatgcttgcattttaaagttctaaaacttaataaattagcatgcttgcatgagatttaagatgtattaaatgttagtggatgatgtggcatctttgcatgttgagctttaagatgtagtgggctttaactttatagaaatataggattaattcctatagaatgtcatgatgcaggatgtcattaataatcctccaagctgttcccttttaacttttttccctgttacttgaaacttgactaaggattctcttcgtattaatgtggattgtgtcactgaccatatggttgtatctttctttcagcgcttcgctgggacttggaatgtttgttgtttttcagtgctttcatggccatggaactcagaatgtctccaacgtgcaaattcttggttgtgatctagaagatggttatttgtttgaaacaatggaagcacttgatgttcccttagcatatacacttgtgagcttgtgttgatagaattgtaaagcttacatatgttttagttctactattattttgaagagggaaatgtgcagctggatgc

SEQ ID NO: 25 - DNA, 799 bp, Oryza sativa
  variation (134): /cultivar=“krr”/note=“ ”c“ replaced with ”t“ ”
aaattcggaatggctagctgttgagagtcattaactccatccatgtgatgggtaacacctactctactctacagtataatactagtgtggtactgatacggtgattatatgctgtactatcattatactactgcggccctgtttggttctatggactaatgtttagctctcacattttaattttaaattagccctcaagaatccaaacaggtgggctaattttgagctaatgtgaattagcccccctcaaaatattagcccctccaagggatgctaatagggttaattttgtgtggggatcatcaaaaagcagctctctctcctctctttctactctctccaacttttagccttgaattagcccatggatccaaatataccaccctaggctaatgtttagcatattaatttatgactaaacattagctcttaaaattagccctggttaatcttaccaacagagcctcgttgtgttacttgtgcacgcgatgcacggacagtttcattctctgtcttcaaaggcttgaagccggcaacatatcgttttcatagacagctattgtaccacaacggtagtaccctacttctccatttctcactcagcttcgtctttaacaacaccgttgtaccatgcttaccatttgcctctctatgaaaataaaaacatcatttcgattttcaaaaatatagctcaactggtttcaactcaactgtctaattaggcgatgtacatatcaacatcggaacgtctataataacttcttaaatctcaaaatatattggttgaatcatcggaggt

SEQ ID NO: 26 - DNA, 709 bp, Oryza sativa
  variation (92): /cultivar=“mth, krr, ghm, yma, hez, ksh, tkh, akk, ank, mtb, ymt, hit, mmm, fom, don, knh, or ssk”/note=“ ”g“ replaced with ”c“ ”
attttcagaacagtcatcatagacatgccaatttactacaagcgaagactggagaggttaggattcaaatagttaataattaactttttttggaatcagtatgctatatatagttaaactttaggagaaagaacattgttgatatgaagacactattgctctaaatatgaacaacacacacaataaatctaagttcggtgtactgaactatcaggtatggacctatattcaaaactaacataggaggccagcacgtggtcatatcccttgatcccgaggtgaaccagttcatatttcaacaagaggggaagttgttccaatcctggtttccagaaaccacactaaacatctttggaaagaagacactcaccacgtataatagaactgctcacaagttgatccggagcttcgtatgcaagctctatggccctgaaaacgtgaaaaaatcactcctgccagaactagagaactccatgagggaaagcttggcgtcatggataggaaaacctagtgtcgaggtgaatgatggcgtgtcaaatgtaagttaacatctgcatttctacataagtattcacaattgcacagtgctcataaaatcatcatgatgttttactatgattaatttctattgtgcagatgatcttcggcctagctgccaaacattgattggcctcgacatcaccattcaggagattgaaaaagacttcag

SEQ ID NO: 27 - DNA, 900 bp, Oryza sativa
  variation (743): /cultivar=“krr”/note=“ ”a“ replaced with ”g“ ”
cacacatcaagcacgatcggaaacgttgtactgtatctcccgcataatgattatggagttggcactcgagatcaagatatcagaatagtatatttttctgttttcaattcttcctctccacagagctcttcctagcctcggcttgtgagagtaagcgcgcctcccagccaactaagaatttgtcggccttcgcaatggttggttgcaggtctgctgttctcaacttttggattgacaaaggaagacctaattaggacattagcagggattcctgtttgcattgcagagtgtgaatcagctgagactcttcctccgctcccacatagatgcatatcattgtgctcttactaaagttgatttgcagacttgttgccctagaaaactcgtctaggcaatgctttagtggtgttacataagcaatcgaagctcgtcaaataatcaatataacattggcatactgcagagctgtacgaggtctctgagggtacagatggtgtccagagcagggtttgctctaagaaggcattggagcacatctgccggatgaacaagtaaggtgacataggatcaccttgacgtagccctctcttacaaccgatccacctgcccaggacttcattgagaaggatagttgactttgaagtctgctacatgttgatcacccaatggatccaagtatcgggaatcctcttgccctcataatatctagcagactttcccatcttatggtttaggaggaattatatggtgtttgacaaatttctaattaaatcttattggcatatatttttcagggaaatatattggttttggttctaagcacaactacaaatgtgtgaaaagtacaagaagctaaaacatactgcatatcaaaacttggagtccacggccataaatata

SEQ ID NO: 28 - DNA, 840 bp, Oryza sativa
  variation (164): /cultivar=“mth, krr, tgr, mnk, yma, hez, ksh, akk, hnh, hit, mmm, fom, or ssk”/note=“ ”t“ replaced with ”c“ ”
ttgaataaaccatggaaaattattacacatataatacattagcgaccaaattgtttcgcccctaactagatgatgccccgcgctttgctgcgggatatatgttagatactggagaaatgaacaaatgatttggattaaaatattatgaaaatggtttgagaattagtatgtttagttttggaatgaagtaaattgtagatataattactatatgcttgcatgttaaactttgtgtgcttaatgggttgatgtggcatgctacatgtaagttttaggagtgctaataaatactatgtttatatgttgagctttaggtgtttagtggacattagctttatagaaagaagagatccctttcctttttcaggtgattttctgtccagtccacctcttttcatctttttttggtataataactctcgtggacgagaatttagagtatttaccatccaattcgtgtgcctcaacttttttacactcaatccgtatgccctctataatactccgtatgacattaggactgctaactagtctatttggtaccactttctcagtttttgatgtatttctcatttcttaactcaatttgtatttcttttatggcattgtggacaattttgcccctaggcccactgtaagatcaaaggaagattgtcgtaggctctccggaccttccattatatttgagagataaagtgaataattcaagtcatcaaacaagcataatgaaacatccgacgcctcgagagagaaatattggagacattgctggacctttagcctgccagtcaacttcagcctaaaatgtgctcgtcaaaccactcgaaggcggcgaccaga

SEQ ID NO: 29 - DNA, 20 bp, Artificial Sequence
  Description of Artificial Sequence an artificially synthesized primer sequence
gcaattgccactggaagaat

SEQ ID NO: 30 - DNA, 20 bp, Artificial Sequence
  Description of Artificial Sequence an artificially synthesized primer sequence
taagttggggaatgcgatgt

SEQ ID NO: 31 - DNA, 20 bp, Artificial Sequence
  Description of Artificial Sequence an artificially synthesized primer sequence
tctgctgcctctgcacatac

SEQ ID NO: 32 - DNA, 21 bp, Artificial Sequence
  Description of Artificial Sequence an artificially synthesized primer sequence
aaaaacgacaccacatcagca

SEQ ID NO: 33 - DNA, 20 bp, Artificial Sequence
  Description of Artificial Sequence an artificially synthesized primer sequence
ggggcgctccttcaaaactt

SEQ ID NO: 34 - DNA, 20 bp, Artificial Sequence
  Description of Artificial Sequence an artificially synthesized primer sequence
ggtttggcacaccacaatgg

SEQ ID NO: 35 - DNA, 20 bp, Artificial Sequence
  Description of Artificial Sequence an artificially synthesized primer sequence
tgcaatgtgccattccatag

SEQ ID NO: 36 - DNA, 20 bp, Artificial Sequence
  Description of Artificial Sequence an artificially synthesized primer sequence
tatgacaaggtgggccctaa

SEQ ID NO: 37 - DNA, 20 bp, Artificial Sequence
  Description of Artificial Sequence an artificially synthesized primer sequence
cgccacagaacggacaaaag

SEQ ID NO: 38 - DNA, 20 bp, Artificial Sequence
  Description of Artificial Sequence an artificially synthesized primer sequence
gaccaatcctttgccgaagc

SEQ ID NO: 39 - DNA, 20 bp, Artificial Sequence
  Description of Artificial Sequence an artificially synthesized primer sequence
ccgatggcagcacaaatctt

SEQ ID NO: 40 - DNA, 20 bp, Artificial Sequence
  Description of Artificial Sequence an artificially synthesized primer sequence
tcagtttggcttgggtgtcc

SEQ ID NO: 41 - DNA, 20 bp, Artificial Sequence
  Description of Artificial Sequence an artificially synthesized primer sequence
ccattggttggtgtggctgt

SEQ ID NO: 42 - DNA, 20 bp, Artificial Sequence
  Description of Artificial Sequence an artificially synthesized primer sequence
tggtcgcggctgataagcta

SEQ ID NO: 43 - DNA, 20 bp, Artificial Sequence
  Description of Artificial Sequence an artificially synthesized primer sequence
tgcgatggagggagtattgg

SEQ ID NO: 44 - DNA, 20 bp, Artificial Sequence
  Description of Artificial Sequence an artificially synthesized primer sequence
tgcgagcgtacaccgctagt

SEQ ID NO: 45 - DNA, 20 bp, Artificial Sequence
  Description of Artificial Sequence an artificially synthesized primer sequence
gcttgaggcacgtcaaaatg

SEQ ID NO: 46 - DNA, 20 bp, Artificial Sequence
  Description of Artificial Sequence an artificially synthesized primer sequence
ttccgtcgttcatgttggtc

SEQ ID NO: 47 - DNA, 20 bp, Artificial Sequence
  Description of Artificial Sequence an artificially synthesized primer sequence
cccacggaaacagccaaaag

SEQ ID NO: 48 - DNA, 20 bp, Artificial Sequence
  Description of Artificial Sequence an artificially synthesized primer sequence
tgctgccatgcaaagaatcg

SEQ ID NO: 49 - DNA, 20 bp, Artificial Sequence
  Description of Artificial Sequence an artificially synthesized primer sequence
attcgaacgggggatccagt

SEQ ID NO: 50 - DNA, 20 bp, Artificial Sequence
  Description of Artificial Sequence an artificially synthesized primer sequence
agcggatcctgctgatgagg

SEQ ID NO: 51 - DNA, 20 bp, Artificial Sequence
  Description of Artificial Sequence an artificially synthesized primer sequence
gtgctgcaaaggggagtatg

SEQ ID NO: 52 - DNA, 20 bp, Artificial Sequence
  Description of Artificial Sequence an artificially synthesized primer sequence
cgccaacctcgtaaatcaaa

SEQ ID NO: 53 - DNA, 25 bp, Artificial Sequence
  Description of Artificial Sequence an artificially synthesized primer sequence
gaacctgaggaccaagtgaaagagt

SEQ ID NO: 54 - DNA, 25 bp, Artificial Sequence
  Description of Artificial Sequence an artificially synthesized primer sequence
ctagagaggagagggagaaggagga

SEQ ID NO: 55 - DNA, 20 bp, Artificial Sequence
  Description of Artificial Sequence an artificially synthesized primer sequence
ataccacaggtgctgcgtga

SEQ ID NO: 56 - DNA, 20 bp, Artificial Sequence
  Description of Artificial Sequence an artificially synthesized primer sequence
tgcgcaactagggattttcc

SEQ ID NO: 57 - DNA, 20 bp, Artificial Sequence
  Description of Artificial Sequence an artificially synthesized primer sequence
ccttgtggtcacacttgcgg

SEQ ID NO: 58 - DNA, 20 bp, Artificial Sequence
  Description of Artificial Sequence an artificially synthesized primer sequence
cggtcttgaggtccagggtg

SEQ ID NO: 59 - DNA, 21 bp, Artificial Sequence
  Description of Artificial Sequence an artificially synthesized primer sequence
tggcatctttgcatgttgagc

SEQ ID NO: 60 - DNA, 20 bp, Artificial Sequence
  Description of Artificial Sequence an artificially synthesized primer sequence
gcatccagctgcacatttcc

SEQ ID NO: 61 - DNA, 20 bp, Artificial Sequence
  Description of Artificial Sequence an artificially synthesized primer sequence
gaatcggttgcaggagaggg

SEQ ID NO: 62 - DNA, 22 bp, Artificial Sequence
  Description of Artificial Sequence an artificially synthesized primer sequence
gcggctatgccatgtttttacc

SEQ ID NO: 63 - DNA, 20 bp, Artificial Sequence
  Description of Artificial Sequence an artificially synthesized primer sequence
cgaccccatgaagcttttgc

SEQ ID NO: 64 - DNA, 21 bp, Artificial Sequence
  Description of Artificial Sequence an artificially synthesized primer sequence
aaatccacgacctccacccct

SEQ ID NO: 65 - DNA, 20 bp, Artificial Sequence
  Description of Artificial Sequence an artificially synthesized primer sequence
ctccctccgctcccagaaat

SEQ ID NO: 66 - DNA, 20 bp, Artificial Sequence
  Description of Artificial Sequence an artificially synthesized primer sequence
attttggtggagcgtcccct

SEQ ID NO: 67 - DNA, 20 bp, Artificial Sequence
  Description of Artificial Sequence an artificially synthesized primer sequence
gcatggatgaccctgctaat

SEQ ID NO: 68 - DNA, 20 bp, Artificial Sequence
  Description of Artificial Sequence an artificially synthesized primer sequence
tgatgccgttgactttttga

SEQ ID NO: 69 - DNA, 20 bp, Artificial Sequence
  Description of Artificial Sequence an artificially synthesized primer sequence
cttgcttgggcaatcgtcaa

SEQ ID NO: 70 - DNA, 20 bp, Artificial Sequence
  Description of Artificial Sequence an artificially synthesized primer sequence
gttgctgacgcgaccagtgt

SEQ ID NO: 71 - DNA, 22 bp, Artificial Sequence
  Description of Artificial Sequence an artificially synthesized primer sequence
gctttccttgtttgaccactcg

SEQ ID NO: 72 - DNA, 21 bp, Artificial Sequence
  Description of Artificial Sequence an artificially synthesized primer sequence
ccattttcatgtcgtggcttg

SEQ ID NO: 73 - DNA, 20 bp, Artificial Sequence
  Description of Artificial Sequence an artificially synthesized primer sequence
acacaagtgtgccattttgc

SEQ ID NO: 74 - DNA, 20 bp, Artificial Sequence
  Description of Artificial Sequence an artificially synthesized primer sequence
tgccaagctacctgagaaca

SEQ ID NO: 75 - DNA, 21 bp, Artificial Sequence
  Description of Artificial Sequence an artificially synthesized primer sequence
cgtgcttggatttttgtaagc

SEQ ID NO: 76 - DNA, 20 bp, Artificial Sequence
  Description of Artificial Sequence an artificially synthesized primer sequence
gcatccagctgcacatttcc

SEQ ID NO: 77 - DNA, 20 bp, Artificial Sequence
  Description of Artificial Sequence an artificially synthesized primer sequence
aaattcggaatggctagctg

SEQ ID NO: 78 - DNA, 20 bp, Artificial Sequence
  Description of Artificial Sequence an artificially synthesized primer sequence
acctccgatgattcaaccaa

SEQ ID NO: 79 - DNA, 21 bp, Artificial Sequence
  Description of Artificial Sequence an artificially synthesized primer sequence
caagcgaagactggagaggtt

SEQ ID NO: 80 - DNA, 20 bp, Artificial Sequence
  Description of Artificial Sequence an artificially synthesized primer sequence
acgtgctggcctcctatgtt

SEQ ID NO: 81 - DNA, 20 bp, Artificial Sequence
  Description of Artificial Sequence an artificially synthesized primer sequence
atcaagcacgatcggaaacg

SEQ ID NO: 82 - DNA, 20 bp, Artificial Sequence
  Description of Artificial Sequence an artificially synthesized primer sequence
atggccgtggactccaagtt

SEQ ID NO: 83 - DNA, 21 bp, Artificial Sequence
  Description of Artificial Sequence an artificially synthesized primer sequence
gaccaaattgtttcgccccta

SEQ ID NO: 84 - DNA, 20 bp, Artificial Sequence
  Description of Artificial Sequence an artificially synthesized primer sequence
gccttcgagtggtttgacga

SEQ ID NO: 85 - DNA, 20 bp, Artificial Sequence
  Description of Artificial Sequence an artificially synthesized primer sequence
aggtcgacacttcggccgtt

SEQ ID NO: 86 - DNA, 21 bp, Artificial Sequence
  Description of Artificial Sequence an artificially synthesized primer sequence
gaacagctgtaataagactga

SEQ ID NO: 87 - DNA, 20 bp, Artificial Sequence
  Description of Artificial Sequence an artificially synthesized primer sequence
gatgcctgcaaagtcccgac

SEQ ID NO: 88 - DNA, 20 bp, Artificial Sequence
  Description of Artificial Sequence an artificially synthesized primer sequence
cgcaaaccatcaacttacaa

SEQ ID NO: 89 - DNA, 22 bp, Artificial Sequence
  Description of Artificial Sequence an artificially synthesized primer sequence
cgattggcagataaagttggat

SEQ ID NO: 90 - DNA, 20 bp, Artificial Sequence
  Description of Artificial Sequence an artificially synthesized primer sequence
tggctagaagtagatgctgc

SEQ ID NO: 91 - DNA, 20 bp, Artificial Sequence
  Description of Artificial Sequence an artificially synthesized primer sequence
aaacaggtgagggaaagatg

SEQ ID NO: 92 - DNA, 20 bp, Artificial Sequence
  Description of Artificial Sequence an artificially synthesized primer sequence
gactgaaaagttgtgtgtgt

SEQ ID NO: 93 - DNA, 25 bp, Artificial Sequence
  Description of Artificial Sequence an artificially synthesized primer sequence
catgaaattattacagaactacaga

SEQ ID NO: 94 - DNA, 20 bp, Artificial Sequence
  Description of Artificial Sequence an artificially synthesized primer sequence
agcacctccccctcctctaa

SEQ ID NO: 95 - DNA, 20 bp, Artificial Sequence
  Description of Artificial Sequence an artificially synthesized primer sequence
ggaactagcccgtgacgctc

SEQ ID NO: 96 - DNA, 20 bp, Artificial Sequence
  Description of Artificial Sequence an artificially synthesized primer sequence
gagagtcgagatgatccaaa

SEQ ID NO: 97 - DNA, 20 bp, Artificial Sequence
  Description of Artificial Sequence an artificially synthesized primer sequence
cagctatagcctagcttgga

SEQ ID NO: 98 - DNA, 23 bp, Artificial Sequence
  Description of Artificial Sequence an artificially synthesized primer sequence
gaagacagcttctgcttgtttgt

SEQ ID NO: 99 - DNA, 20 bp, Artificial Sequence
  Description of Artificial Sequence an artificially synthesized primer sequence
aacgtcatggacgatccgct

SEQ ID NO: 100 - DNA, 20 bp, Artificial Sequence
  Description of Artificial Sequence an artificially synthesized primer sequence
gccatgaaagcactgaaaaa

SEQ ID NO: 101 - DNA, 20 bp, Artificial Sequence
  Description of Artificial Sequence an artificially synthesized primer sequence
ttgagttcttggggatttgt

SEQ ID NO: 102 - DNA, 25 bp, Artificial Sequence
  Description of Artificial Sequence an artificially synthesized primer sequence
tgttacaagcaaagcatgaggaatg

SEQ ID NO: 103 - DNA, 20 bp, Artificial Sequence
  Description of Artificial Sequence an artificially synthesized primer sequence
agctcgagctcgaagatggc

SEQ ID NO: 104 - DNA, 28 bp, Artificial Sequence
  Description of Artificial Sequence an artificially synthesized primer sequence
caaacatttaaaatataaatcatgaata

SEQ ID NO: 105 - DNA, 25 bp, Artificial Sequence
  Description of Artificial Sequence an artificially synthesized primer sequence
taagcccccggccgaaccggcaaag

SEQ ID NO: 106 - DNA, 20 bp, Artificial Sequence
  Description of Artificial Sequence an artificially synthesized primer sequence
gactacaatcttccactcca

SEQ ID NO: 107 - DNA, 26 bp, Artificial Sequence
  Description of Artificial Sequence an artificially synthesized primer sequence
tgtgaactacactatttagttgctta

SEQ ID NO: 108 - DNA, 21 bp, Artificial Sequence
  Description of Artificial Sequence an artificially synthesized primer sequence
ctgggacttggaatgtttgtt

SEQ ID NO: 109 - DNA, 22 bp, Artificial Sequence
  Description of Artificial Sequence an artificially synthesized primer sequence
gctaatgtgaattagcccccct

SEQ ID NO: 110 - DNA, 28 bp, Artificial Sequence
  Description of Artificial Sequence an artificially synthesized primer sequence
agtttaactatatatagcatactgattc

SEQ ID NO: 111 - DNA, 23 bp, Artificial Sequence
  Description of Artificial Sequence an artificially synthesized primer sequence
catcttatggtttaggaggaatt

SEQ ID NO: 112 - DNA, 21 bp, Artificial Sequence
  Description of Artificial Sequence an artificially synthesized primer sequence
gtctatttggtaccactttct

SEQ ID NO: 113 - DNA, 20 bp, Artificial Sequence
  Description of Artificial Sequence an artificially synthesized primer sequence
accgggtagggaaacaaaac

SEQ ID NO: 114 - DNA, 20 bp, Artificial Sequence
  Description of Artificial Sequence an artificially synthesized primer sequence
aataatacttcggcgcatcg

(19) United States
(12) Patent Application Publication
(10) Pub. No.: US 2006/0035227 A1
(43) Pub. Date: Feb. 16, 2006

(54) Methods for distinguishing rice varities

(76) Inventors: Yuzo Minobe, Tsukuba-shi, Ibaraki (JP); Lisa Monna, Tsukuba-shi, Ibaraki (JP); Junko Suzuki, Arakaira , Tokyo (JP); Rieko Ohta, Abiko-shi, Chiba (JP); Hiroshi Nemoto, Tsukuba-shi (JP); Osamu Ideta, Tsukuba-shi, Ibaraki (JP)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/517,543

(22) PCT Filed: Jun. 10, 2003
(86) PCT No.: PCT/JP03/07332

§ 371 Date: Jul. 20, 2005

(30) Foreign Application Priority Data
Jun. 10, 2002 (JP) 2002-168875

(51) Int. Cl. C12Q 1/68

(52) U.S. Cl. 435/6

(57) ABSTRACT

Polymorphisms were searched in 24 varieties with large planted acreages in Japan, and the polymorphic sites were compared among the varieties. Thus, polymorphic markers that can be used to distinguish varieties in a simple and quick manner were obtained. The markers showed distinct patterns for each of the varieties, demonstrating that their combination would enable the varieties to be distinguished. Thus, the inventors succeeded in obtaining molecular markers that can distinguish 24 rice varieties. The use of these markers enables closely related rice varieties to be distinguished and identified at the DNA level.

### TECHNICAL FIELD

This invention relates to methods for distinguishing between rice varieties.

### BACKGROUND ART

Traditionally, varieties of rice plants or rice have been distinguished by cultivation traits (e.g., height, number of tillers, days to heading), grain traits before/after polishing (e.g., grain shape, weight, and whiteness), and cooking qualities (e.g., taste). In addition, sorting using molecular genetic analysis such as RFLP (restriction fragment length polymorphism) and CAPS (cleaved amplified polymorphic sequence) has become feasible. However, the eyes of an experienced breeder are required to distinguish varieties by their cultivation traits, and this is not something that just anyone can judge. In addition, statistical analysis of the traits of unpolished or polished rice is required, and a certain quantity of rice is required to determine cooking qualities. Thus, it is impossible to distinguish each individual rice grain. In principle, molecular genetic analysis has solved these problems; however, in fact, while effective for distinguishing between remotely related varieties, such analysis is troublesome for closely related varieties, because it is difficult to obtain established molecular markers.

By definition, single nucleotide polymorphisms (SNPs) are single nucleotide differences existing in DNA nucleotide sequences. In practice, they often include SSR (simple sequence repeats) and insertion or deletion mutations. It is no exaggeration to say that SNPs cause all genetic differences detectable using molecular markers such as RFLP and CAPS, and all genetic differences reflected in phenotypes and such. SNP studies and SNP assay systems have made remarkable progress in recent years. Currently, an assay system has been developed that allows all steps, from PCR to a result, to be carried out in a 96-well plate, with no need for electrophoresis, enabling remarkably efficient genotyping compared to traditional molecular markers.

Recently, the reliability of labeling requirements in the food industry has become an issue, and rice is no exception. For example, the amount of rice being sold as Koshihikari exceeds the national production of Koshihikari. Thus, the possibility of false disclosure in the rice market cannot be denied, and both consumers and sellers desire assays that accurately distinguish polished rice varieties, and determine blend ratios.

### DISCLOSURE OF THE INVENTION

The present invention has been accomplished by considering the above circumstances. An objective of the present invention is to provide novel methods that enable rice varieties to be quickly and easily distinguished. More specifically, the present invention aims to provide methods for efficiently distinguishing between rice varieties by using polymorphic markers.

In order to solve these problems, the present inventors carried out intensive studies. First, using the rice genomic sequence, primers for amplifying 800 bp to 1 kbp fragments from genomic DNA were designed by selecting mainly putative intergenic regions for those chromosomal regions for which rice genomic nucleotide sequences were publicly available, and by using the sequence of RFLP marker probes and the like for other regions. The designed primers were used in PCR amplifications, with DNA extracted by a simple method from rice varieties Nipponbare, Koshihikari, Kasalath, Guang-lu-ai 4 (G4, below), Kitaake, and a wild rice (Oryza rufipogon, W1943), as a template, to prepare templates for sequencing reactions. The templates were then subjected to cycle sequencing, and samples for sequencing were prepared. The resulting sequence data was compared between varieties, to search for single nucleotide substitution polymorphisms. Each variety was sequenced at least twice with each primer, and only definite cases were deemed to be polymorphisms.

The nucleotide sequences at positions found to be polymorphic between Nipponbare and Koshihikari, and between Nipponbare and Kitaake, were examined by performing PCR and sequencing as described above, using templates of genomic DNA extracted by a simple method from Nipponbare, Hatsushimo, Mutsuhomare, Yukinosei, Kirara 397, Tsugaruroman, Gohyakumangoku, Morinokumasan, Yumeakari, Hanaechizen, Koshihikari, Tsukinohikari, Akitakomachi, Asanohikari, Aichinokaori, Matsuribare, Hinohikari, Yumetsukushi, Hitomebore, Manamusume, Fusaotome, Dontokoi, Kinuhikari, and Sasanishiki. The nucleotide sequences at the polymorphic sites were compared between the varieties.

Next, primers for detecting SNPs that are useful for distinguishing varieties were designed, and single nucleotide terminator reactions were performed using an AcycloPrime-FP kit (Perkin Elmer) to prepare samples for genotyping. Genotyping was performed by using ARVO (Perkin Elmer) to measure fluorescence polarization.

The results showed that the markers generated around those positions determined by sequencing to be SNPs displayed distinct patterns, and that they could be used in combination to sort the varieties into different groups. Thus, the inventors succeeded in obtaining polymorphic markers that could be used to distinguish between 24 rice varieties.

As described above, the inventors searched for SNPs in 24 rice varieties with a large planted acreage in Japan, and obtained polymorphic markers that enabled the varieties to be distinguished in a quick and simple manner. They thus accomplished novel methods for distinguishing rice varieties using the polymorphic markers. The methods of the invention enable closely related varieties to be distinguished and identified at the DNA level.

Thus, the present invention relates to novel methods for distinguishing rice varieties in a quick and simple manner, and more specifically, it provides:

[1] a method of distinguishing between rice varieties, comprising the following steps (a) and (b):

(a) determining the type of a nucleotide at-a position according to any of the following (1) to (28) in the rice genome, or a nucleotide on the complementary strand that composes a base pair with the nucleotide at the position:
(1) position 593 in the nucleotide sequence of SEQ ID NO: 1,
(2) position 304 in the nucleotide sequence of SEQ ID NO: 2,
(3) position 450 in the nucleotide sequence of SEQ ID NO: 3,
(4) position 377 in the nucleotide sequence of SEQ ID NO: 4,
(5) position 163 in the nucleotide sequence of SEQ ID NO: 5,
(6) position 624 in the nucleotide sequence of SEQ ID NO: 6,
(7) position 534 in the nucleotide sequence of SEQ ID NO: 7,
(8) position 358 in the nucleotide sequence of SEQ ID NO: 8,
(9) position 475 in the nucleotide sequence of SEQ ID NO: 9,
(10) position 323 in the nucleotide sequence of SEQ ID NO: 10,
(11) position 612 in the nucleotide sequence of SEQ ID NO: 11,
(12) position 765 in the nucleotide sequence of SEQ ID NO: 12,
(13) position 571 in the nucleotide sequence of SEQ ID NO: 13,
(14) position 660 in the nucleotide sequence of SEQ ID NO: 14,
(15) position 223 in the nucleotide sequence of SEQ ID NO: 15,
(16) position 247 in the nucleotide sequence of SEQ ID NO: 16,
(17) position 163 in the nucleotide sequence of SEQ ID NO: 17,
(18) position 421 in the nucleotide sequence of SEQ ID NO: 18,
(19) position 178 in the nucleotide sequence of SEQ ID NO: 19,
(20) position 141 in the nucleotide sequence of SEQ ID NO: 20,
(21) position 480 in the nucleotide sequence of SEQ ID NO: 21,
(22) position 481 in the nucleotide sequence of SEQ ID NO: 22,
(23) position 131 in the nucleotide sequence of SEQ ID NO: 23,
(24) position 510 in the nucleotide sequence of SEQ ID NO: 24,
(25) position 248 in the nucleotide sequence of SEQ ID NO: 25,
(26) position 92 in the nucleotide sequence of SEQ ID NO: 26,
(27) position 743 in the nucleotide sequence of SEQ ID NO: 27, and
(28) position 552 in the nucleotide sequence of SEQ ID NO: 28, and

(b) relating the type of the nucleotide determined in step (a) to a variety of rice;

[2] the method of [1], which distinguishes the type of a nucleotide by using a polymorphic marker characterized by a mutation of any of the following (1) to (28) in the rice genome:
(1) the nucleotide at position 593 in the nucleotide sequence of SEQ ID NO: 1 is T,
(2) the nucleotide at position 304 in the nucleotide sequence of SEQ ID NO: 2 is T,
(3) the nucleotide at position 450 in the nucleotide sequence of SEQ ID NO: 3 is A,
(4) the nucleotide at position 377 in the nucleotide sequence of SEQ ID NO: 4 is C,
(5) the nucleotide at position 163 in the nucleotide sequence of SEQ ID NO: 5 is C,
(6) the nucleotide at position 624 in the nucleotide sequence of SEQ ID NO: 6 is C,
(7) the nucleotide at position 534 in the nucleotide sequence of SEQ ID NO: 7 is C,
(8) the nucleotide at position 358 in the nucleotide sequence of SEQ ID NO: 8 is G,
(9) the nucleotide at position 475 in the nucleotide sequence of SEQ ID NO: 9 is G,
(10) the nucleotide at position 323 in the nucleotide sequence of SEQ ID NO: 10 is A,
(11) the nucleotide at position 612 in the nucleotide sequence of SEQ ID NO: 11 is A,
(12) the nucleotide at position 765 in the nucleotide sequence of SEQ ID NO: 12 is T,
(13) the nucleotide at position 571 in the nucleotide sequence of SEQ ID NO: 13 is T,
(14) the nucleotide at position 660 in the nucleotide sequence of SEQ ID NO: 14 is G,
(15) the nucleotide at position 223 in the nucleotide sequence of SEQ ID NO: 15 is A,
(16) the nucleotide at position 247 in the nucleotide sequence of SEQ ID NO: 16 is A,
(17) the nucleotide at position 163 in the nucleotide sequence of SEQ ID NO: 17 is A,
(18) the nucleotide at position 421 in the nucleotide sequence of SEQ ID NO: 18 is C,
(19) the nucleotide at position 178 in the nucleotide sequence of SEQ ID NO: 19 is G,
(20) the nucleotide at position 141 in the nucleotide sequence of SEQ ID NO: 20 is G,
(21) the nucleotide at position 480 in the nucleotide sequence of SEQ ID NO: 21 is C,
(22) the nucleotide at position 481 in the nucleotide sequence of SEQ ID NO: 22 is C,
(23) the nucleotide at position 131 in the nucleotide sequence of SEQ ID NO: 23 is C,
(24) the nucleotide at position 510 in the nucleotide sequence of SEQ ID NO: 24 is A,
(25) the nucleotide at position 248 in the nucleotide sequence of SEQ ID NO: 25 is T,
(26) the nucleotide at position 92 in the nucleotide sequence of SEQ ID NO: 26 is C,
(27) the nucleotide at position 743 in the nucleotide sequence of SEQ ID NO: 27 is G, and
(28) the nucleotide at position 552 in the nucleotide sequence of SEQ ID NO: 28 is T;

[3] the method of [1] or [2], comprising the following steps (a) to (c):

(a) preparing DNA from a test rice,

(b) amplifying a DNA comprising a nucleotide in a position of any of (1) to (28) of [1], or a nucleotide in the complementary strand composing a base pair with the nucleotide at the position, and

(c) determining the nucleotide sequence of the amplified DNA;

[4] the method of [1] or [2], comprising the following steps (a) to (d):

(a) preparing DNA from a test rice,

(b) digesting the prepared DNA with a restriction enzyme,

(c) fractionating the DNA fragments by size, and

(d) comparing the size of the detected DNA fragment with a control;

[5] the method of [1] or [2], comprising the following steps (a) to (e):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) digesting the amplified DNA with a restriction enzyme,

(d) fractionating the DNA fragments by size, and

(e) comparing the size of the detected DNA fragment with a control;

[6] the method of [1] or [2], comprising the following steps (a) to (e):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) denaturing the amplified DNA into single-stranded DNAs,

(d) fractionating the denatured single-stranded DNA on a non-denaturing gel, and

(e) comparing the mobility of the fractionated single-stranded DNA on the gel with a control;

[7] the method of [1] or [2], comprising the following steps (a) to (f):

(a) preparing DNA from a test rice,

(b) synthesizing two different oligonucleotide probes labeled with a reporter fluorescence dye and quencher fluorescence dye, where an oligonucleotide is complementary to a proximal nucleotide sequence comprising a nucleotide in a position of any of (1) to (28) of [1], or a nucleotide in the complementary strand composing a base pair with the nucleotide at the position,

(c) hybridizing the DNA prepared in step (a) with the probe synthesized in step (b),

(d) amplifying a DNA comprising a nucleotide in a position of any of (1) to (28) of [1], or a nucleotide in the complementary strand composing a base pair with the nucleotide at the position,

(e) detecting the emission of reporter fluorescence, and

(f) comparing the emission of reporter fluorescence detected in step (e) with a control;

[8] the method of [1] or [2], comprising the following steps (a) to (h):

(a) preparing DNA from a test rice,

(b) synthesizing a probe in which a sequence complementary to the 3′-flanking nucleotide sequence comprising a nucleotide in a position of any of (1) to (28) of [1], or a nucleotide in the complementary strand composing a base pair with the nucleotide at the position, is combined with a totally unrelated sequence,

(c) synthesizing a probe that is complementary to the 5′-flanking region comprising a nucleotide in a position of any of (1) to (28) of [1], or a nucleotide in the complementary strand composing a base pair with the nucleotide at the position,

(d) hybridizing the probe synthesized in step (c) with the DNA prepared in step (a),

(e) digesting the hybridized DNA in step (d) with a single-stranded DNA cleaving enzyme, and dissociating a part of the probe synthesized in step (b),

(f) hybridizing the dissociated probe in step (e) with a probe for detection,

(g) enzymatically digesting the hybridized DNA in step (f), and measuring the fluorescence intensity thus generated, and

(h) comparing the fluorescence intensity measured in step (g) with a control;

[9] the method of [1] or [2], comprising the following steps (a) to (f):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) denaturing the amplified DNA into single-stranded DNAs,

(d) separating only one strand from the denatured single-stranded DNAs,

(e) performing an elongation reaction from near a position of any of (1) to (28) of [1], or a nucleotide in the complementary strand composing a base pair with the nucleotide at the position, whereby the reaction elongates one nucleotide at a time, then enzymatically illuminating the generated pyrophosphate, and measuring the intensity of the illumination, and

(f) comparing the fluorescence intensity measured in step (e) with a control;

[10] the method of [1] or [2], comprising the following steps (a) to (f):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) synthesizing a probe complementary to a nucleotide sequence comprising a sequence covering up to a nucleotide adjacent to a position of any of (1) to (28) of [1], or a nucleotide in the complementary strand composing a base pair with the nucleotide at the position,

(d) performing a single nucleotide extension reaction in the presence of fluorescently labeled nucleotides, using the DNA amplified in step (b) as a template, and the primer synthesized in step (c),

(e) measuring the fluorescence polarization, and

(f) comparing the fluorescence polarization measured in step (e) with a control;

[11] the method of [1] or [2], comprising the following steps (a) to (f):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) synthesizing a primer complementary to a nucleotide sequence comprising a sequence covering up to the nucleotide adjacent to a position of any of (1) to (28) of [1], or a nucleotide in the complementary strand composing a base pair with the nucleotide at the position,

(d) performing a single nucleotide extension reaction in the presence of fluorescently labeled nucleotides, using the DNA amplified in step (b) as a template, and the primer synthesized in step (c),

(e) determining the nucleotide variety used in the reaction of step (d) using a sequencer, and

(f) comparing the nucleotide determined in step (e) with a control;

[12] the method of [1] or [2], comprising the following steps (a) to (d):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) measuring the molecular weight of the DNA amplified in step (b) using a mass spectrometer, and

(d) comparing the molecular weight measured in step (c) with a control;

[13] the method of [1] or [2], comprising the following steps (a) to (f):

(a) preparing DNA from a test rice,

(b) amplifying a DNA comprising a nucleotide in a position of any of (1) to (28) of [1], or a nucleotide in the complementary strand composing a base pair with the nucleotide at the position,

(c) providing a substratum on which a nucleotide probe is immobilized,

(d) contacting the DNA of step (b) with the substratum of step (c),

(e) detecting the strength of hybridization between the DNA and the nucleotide probe immobilized on the substratum, and

(f) comparing the strength detected in step (e) with a control;

[14] the method of any of [1] to [13], further comprising the following steps (a) and (b):

(a) disrupting a rice seed in an alkaline aqueous solvent, and

(b) extracting rice genomic DNA from the seed disrupted in step (a);

[15] the method of [14], wherein the rice seed is polished;

[16] a primer for distinguishing between rice varieties (or a reagent for distinguishing between rice varieties), wherein the primer is (a) an oligonucleotide for amplification of a DNA region comprising a nucleotide in a position of any of (1) to (28) of [1] in the rice genome, or a nucleotide in the complementary strand composing a base pair with the nucleotide at the position, or (b) an oligonucleotide comprising a nucleotide sequence complementary to a sequence covering up to a nucleotide adjacent to a position of any of (1) to (28) of [1] in the rice genome, or a nucleotide in the complementary strand composing a base pair with the nucleotide at the position;

[17] an oligonucleotide for distinguishing between rice varieties (or a reagent for distinguishing between rice varieties), wherein the oligonucleotide hybridizes with a DNA region comprising a nucleotide in a position of any of (1) to (28) of [1], or a nucleotide in the complementary strand composing a base pair with the nucleotide at the position, comprising at least 15 nucleotides;

[18] a kit for distinguishing between rice varieties, comprising the oligonucleotide of [16] or [17]; and

[19] the kit of [18], further comprising an alkaline aqueous solvent.

The inventors analyzed the genomic sequences of 24 rice varieties, and thus discovered polymorphic markers that enable the rice varieties to be accurately distinguished. SEQ ID NOs: 1 to 28 show the DNA regions in the rice genome that comprise the polymorphic sites identified by the inventors. The positions of each of the polymorphisms are shown in FIGS. 1 to 29, and in Tables 8 and 9.

The present invention provides methods for distinguishing rice varieties. In the methods, the type of a nucleotide at a polymorphic site, identified by the inventors in the genome of 24 rice varieties, is first determined. More specifically, a nucleotide at any of the following positions (1) to (28), or a nucleotide composing a base pair with the above nucleotide at a position in the complementary strand, is determined in the rice genome (step (A)).

(1) Position 593 in the nucleotide sequence of SEQ ID NO: 1,

(2) Position 304 in the nucleotide sequence of SEQ ID NO: 2,

(3) Position 450 in the nucleotide sequence of SEQ ID NO: 3,

(4) Position 377 in the nucleotide sequence of SEQ ID NO: 4,

(5) Position 163 in the nucleotide sequence of SEQ ID NO: 5,

(6) Position 624 in the nucleotide sequence of SEQ ID NO: 6,

(7) Position 534 in the nucleotide sequence of SEQ ID NO: 7,

(8) Position 358 in the nucleotide sequence of SEQ ID NO: 8,

(9) Position 475 in the nucleotide sequence of SEQ ID NO: 9,

(10) Position 323 in the nucleotide sequence of SEQ ID NO: 10,

(11) Position 612 in the nucleotide sequence of SEQ ID NO: 11,

(12) Position 765 in the nucleotide sequence of SEQ ID NO: 12,

(13) Position 571 in the nucleotide sequence of SEQ ID NO: 13,

(14) Position 660 in the nucleotide sequence of SEQ ID NO: 14,

(15) Position 223 in the nucleotide sequence of SEQ ID NO: 15,

(16) Position 247 in the nucleotide sequence of SEQ ID NO: 16,

(17) Position 163 in the nucleotide sequence of SEQ ID NO: 17,

(18) Position 421 in the nucleotide sequence of SEQ ID NO: 18,

(19) Position 178 in the nucleotide sequence of SEQ ID NO: 19,

(20) Position 141 in the nucleotide sequence of SEQ ID NO: 20,

(21) Position 480 in the nucleotide sequence of SEQ ID NO: 21,

(22) Position 481 in the nucleotide sequence of SEQ ID NO: 22,

(23) Position 131 in the nucleotide sequence of SEQ ID NO: 23,

(24) Position 510 in the nucleotide sequence of SEQ ID NO: 24,

(25) Position 248 in the nucleotide sequence of SEQ ID NO: 25,

(26) Position 92 in the nucleotide sequence of SEQ ID NO: 26,

(27) Position 743 in the nucleotide sequence of SEQ ID NO: 27,

(28) Position 552 in the nucleotide sequence of SEQ ID NO: 28.

With information such as the nucleotide sequences and polymorphic sites disclosed herein, it is normally easy for one skilled in the art to appropriately identify an actual position on the genome that corresponds to a polymorphic site. For example, the genomic position of a polymorphism of this invention can be identified by referring to publicly available genome databases and the like. That is, even if a slight difference is found between a nucleotide sequence in the Sequence Listing and an actual genomic sequence, the polymorphic sites of this invention on the actual genome can be precisely identified by performing homology searches and such over the genomic sequence, using a nucleotide sequence shown in the Sequence Listing.

Normally, genomic DNA is composed of complementary double-stranded DNA. Therefore, herein, even when the DNA sequence of one strand is disclosed for descriptive purposes, it should be naturally assumed that its complementary sequence (nucleotide) is also disclosed. When the DNA sequence (nucleotide) of one strand is known, its complementary sequence (nucleotide) is obvious to those skilled in the art.

Herein, a “polymorphism” is not limited to single nucleotide polymorphisms (SNPs), comprising a mutation such as the substitution, deletion, and insertion of a single nucleotide. “Polymorphism” also includes those of several continuous nucleotides. A “polymorphic marker” is herein defined as information on a nucleotide mutation at a polymorphic site (polymorphic mutation). More specifically, a polymorphic marker of this invention refers to information on a mutation in a nucleotide sequence, identified by comparing the genomic sequence of rice variety Nipponbare with that of another variety, that can be used to distinguish the rice varieties. Herein, the polymorphic markers used to determine the type of a nucleotide are preferably the polymorphic markers described in the following (1′) to (28′). Thus, in a preferred embodiment of this invention, rice varieties are distinguished by using the polymorphic markers described in the following (1′) to (28′):

(1′) The nucleotide at position 593 in the nucleotide sequence of SEQ ID NO: 1 is T. More specifically, the nucleotide at position 593 in the nucleotide sequence of SEQ ID NO: 1 in the Nipponbare genome comprises a C to T substitution.

(2′) The nucleotide at position 304 in the nucleotide sequence of SEQ ID NO: 2 is T. More specifically, the nucleotide at position 304 in the nucleotide sequence of SEQ ID NO: 2 in the Nipponbare genome comprises an A to T substitution.

(3′) The nucleotide at position 450 in the nucleotide sequence of SEQ ID NO: 3 is A. More specifically, the nucleotide at position 450 in the nucleotide sequence of SEQ ID NO: 3 in the Nipponbare genome comprises a G to A substitution.

(4′) The nucleotide at position 377 in the nucleotide sequence of SEQ ID NO: 4is C. More specifically, the nucleotide at position 377 in the nucleotide sequence of SEQ ID NO: 4 in the Nipponbare genome comprises a T to C substitution.

(5′) The nucleotide at position 163 in the nucleotide sequence of SEQ ID NO: 5 is C. More specifically, the nucleotide at position 163 in the nucleotide sequence of SEQ ID NO: 5 in the Nipponbare genome comprises a T to C substitution.

(6′) The nucleotide at position 624 in the nucleotide sequence of SEQ ID NO: 6 is C. More specifically, the nucleotides at positions 624 to 626 in the nucleotide sequence of SEQ ID NO: 6 in the Nipponbare genome are deleted.

(7′) The nucleotide at position 534 in the nucleotide sequence of SEQ ID NO: 7 is C. More specifically, the nucleotide at position 534 in the nucleotide sequence of SEQ ID NO: 7 in the Nipponbare genome comprises an A to C substitution.

(8′) The nucleotide at position 358 in the nucleotide sequence of SEQ ID NO: 8 is G. More specifically, GT is inserted between the nucleotides at positions 358 and 389 in the nucleotide sequence of SEQ ID NO: 8 in the Nipponbare genome.

(9′) The nucleotide at position 475 in the nucleotide sequence of SEQ ID NO: 9 is G. More specifically, the nucleotide at position 475 in the nucleotide sequence of SEQ ID NO: 9 in the Nipponbare genome comprises a T to G substitution.

(10′) The nucleotide at position 323 in the nucleotide sequence of SEQ ID NO: 10 is A. More specifically, the nucleotide at position 323 in the nucleotide sequence of SEQ ID NO: 10 in the Nipponbare genome comprises a G to A substitution.

(11′) The nucleotide at position 612 in the nucleotide sequence of SEQ ID NO: 11 is A. More specifically, the nucleotides at positions 612 and 613 in the nucleotide sequence of SEQ ID NO: 11 in the Nipponbare genome are substituted from CA to AG.

(12′) The nucleotide at position 765 in the nucleotide sequence of SEQ ID NO: 12 is T. More specifically, the nucleotide at position 765 in the nucleotide sequence of SEQ ID NO: 12 in the Nipponbare genome comprises a G to T substitution.

(13′) The nucleotide at position 571 in the nucleotide sequence of SEQ ID NO: 13 is T. More specifically, the nucleotide at position 571 in the nucleotide sequence of SEQ ID NO: 13 in the Nipponbare genome comprises a G to T substitution.

(14′) The nucleotide at position 660 in the nucleotide sequence of SEQ ID NO: 14 is G. More specifically, the nucleotide at position 660 in the nucleotide sequence of SEQ ID NO: 14 in the Nipponbare genome comprises an A to G substitution.

(15′) The nucleotide at position 223 in the nucleotide sequence of SEQ ID NO: 15 is A. More specifically, the nucleotide at position 223 in the nucleotide sequence of SEQ ID NO: 15 in the Nipponbare genome comprises a G to A substitution.

(16′) The nucleotide at position 247 in the nucleotide sequence of SEQ ID NO: 16 is A. More specifically, the nucleotide at position 247 in the nucleotide sequence of SEQ ID NO: 16 in the Nipponbare genome comprises a G to A substitution.

(17′) The nucleotide at position 163 in the nucleotide sequence of SEQ ID NO: 17 is A. More specifically, the nucleotide at position 163 in the nucleotide sequence of SEQ ID NO: 17 in the Nipponbare genome comprises a G to A substitution.

(18′) The nucleotide at position 421 in the nucleotide sequence of SEQ ID NO: 18 is C. More specifically, the nucleotide at position 421 in the nucleotide sequence of SEQ ID NO: 18 in the Nipponbare genome comprises an A to C substitution.

(19′) The nucleotide at position 178 in the nucleotide sequence of SEQ ID NO: 19 is G. More specifically, the nucleotide at position 178 in the nucleotide sequence of SEQ ID NO: 19 in the Nipponbare genome is deleted.

(20′) The nucleotide at position 141 in the nucleotide sequence of SEQ ID NO: 20 is G. More specifically, the nucleotide at position 141 in the nucleotide sequence of SEQ ID NO: 20 in the Nipponbare genome comprises an A to G substitution.

(21′) The nucleotide at position 480 in the nucleotide sequence of SEQ ID NO: 21 is C. More specifically, the nucleotide at position 480 in the nucleotide sequence of SEQ ID NO: 21 in the Nipponbare genome comprises a T to C substitution.

(22′) The nucleotide at position 481 in the nucleotide sequence of SEQ ID NO: 22 is C. More specifically, the nucleotide at position 481 in the nucleotide sequence of SEQ ID NO: 22 in the Nipponbare genome comprises a T to C substitution.

(23′) The nucleotide at position 131 in the nucleotide sequence of SEQ ID NO: 23 is C. More specifically, the nucleotide at position 131 in the nucleotide sequence of SEQ ID NO: 23 in the Nipponbare genome comprises a G to C substitution.

(24′) The nucleotide at position 510 in the nucleotide sequence of SEQ ID NO: 24 is A. More specifically, the nucleotide at position 510 in the nucleotide sequence of SEQ ID NO: 24 in the Nipponbare genome comprises a G to A substitution.

(25′) The nucleotide at position 248 in the nucleotide sequence of SEQ ID NO: 25 is T. More specifically, the nucleotide at position 248 in the nucleotide sequence of SEQ ID NO: 25 in the Nipponbare genome comprises a C to T substitution.

(26′) The nucleotide at position 92 in the nucleotide sequence of SEQ ID NO: 26 is C. More specifically, the nucleotide at position 92 in the nucleotide sequence of SEQ ID NO: 26 in the Nipponbare genome comprises a G to C substitution.

(27′) The nucleotide at position 743 in the nucleotide sequence of SEQ ID NO: 27 is G. More specifically, the nucleotide at position 743 in the nucleotide sequence of SEQ ID NO: 27 in the Nipponbare genome comprises an A to G substitution.

(28′) The nucleotide at position 552 in the nucleotide sequence of SEQ ID NO: 28 is T. More specifically, the nucleotide at position 552 in the nucleotide sequence of SEQ ID NO: 28 in the Nipponbare genome comprises a C to T substitution.

Herein, “determining the type of nucleotide” normally means determining the nucleotide sequence at a position described in any of the above (1) to (28) on the genome of a rice whose variety is being distinguished (also described below as a “test rice”). However, it may not be necessary to specifically determine the actual species of the nucleotide. Even when a nucleotide sequence at any of the above positions (1) to (28) is not specifically determined in the genome of a test rice, it is possible to distinguish the rice variety by examining whether or not it is identical to that of Nipponbare.

Next, in the methods of this invention, a nucleotide sequence determined in the above step (A) is related to the rice varieties (step (B)).

The rice varieties that can be distinguished by the methods of this invention are as follows (the name of each variety may be abbreviated herein, as shown in the parentheses) : Nipponbare (nhb), Hatsushimo (hts), Mutsuhomare (mth), Yukinosei (yki), Kirara 397 (krr), Tsugaruroman (tgr), Gohyakumangoku (ghm), Morinokumasan (mnk), Yumeakari (yma), Hanaechizen (hez), Koshihikari (ksh), Tsukinohikari (tkh), Akitakomachi (akk), Asanohikari (ash), Aichinokaori (ank), Matsuribare (mtb), Hinohikari (hnh), Yumetsukushi (ymt), Hitomebore (hit), Manamusume (mmm), Fusaotome (fom), Dontokoi (don), Kinuhikari (knh), Sasanishiki (ssk), Akebono (akb), and Goropikari (grp).

The methods of this invention for distinguishing rice varieties may be normally used to identify the name of a rice of unknown variety, selected from the above varieties, or to determine whether a rice belongs to one of the above varieties.

The present inventors determined the nucleotide sequences at positions described in the above (1) to (28) in the genome of the above rice varieties, and obtained polymorphic markers. Table 1 shows the details of the polymorphic markers (the names of the polymorphic markers, and the nucleotide sequences at the above positions (1) to (28) for each of the rice varieties).

TABLE 1

| | SEQ | | SNP Detection | | | | | | | |
|---|---|---|---|---|---|---|---|---|---|---|
| Marker | ID | | Nipponbare | Hatsushimo | Mutsuhomare | Yukinosei | Kirara 397 | Tsugaruroman | Gohyakumangoku | Morinokumasan |
| name | NO: | Position | nhb | hts | mth | yki | krr | tgr | ghm | mnk |
| S0015 | 1 | 593 | C | C | C | C | T | T | C | T |
| S0040 | 2 | 304 | T | T | T | A | A | A | A | A |
| S0279 | 3 | 450 | C | T | T | T | T | T | T | T |
| S0044 | 4 | 377 | T | T | T | T | T | T | T | T |
| S0252 | 5 | 163 | T | C | C | C | C | T | T | T |
| S0109 | 6 | 624 | T | T | C | T | C | C | C | C |
| S0115 | 7 | 534 | T | G | G | G | G | T | T | G |
| S0107 | 8 | 358 | A | A | G | G | G | G | G | G |
| S0126 | 9 | 475 | T | T | T | G | T | T | T | T |
| S0124 | 10 | 323 | G | G | G | G | A | G | G | G |
| S0146 | 11 | 612 | C | C | C | C | A | A | C | C |
| S0135 | 12 | 765 | G | G | G | G | G | G | T | G |
| S0155 | 13 | 571 | G | G | T | G | T | T | T | T |
| S0161 | 14 | 660 | A | G | G | A | A | G | A | A |
| S0177 | 15 | 223 | G | A | A | A | A | A | A | A |
| S0178 | 16 | 247 | C | C | C | C | T | C | T | C |
| S0174 | 17 | 163 | G | G | G | G | G | A | G | A |
| S0185 | 18 | 421 | A | A | A | A | A | C | C | C |
| S0208 | 19 | 178 | C | G | G | G | G | G | G | G |
| S0007 | 20 | 141 | A | A | A | G | G | A | G | A |
| S0070 | 21 | 480 | A | A | G | A | A | A | G | A |
| S0310 | 22 | 481 | T | T | T | T | C | C | C | C |
| S0375 | 23 | 131 | G | G | C | G | G | C | G | C |
| S0346 | 24 | 510 | G | G | G | G | A | G | A | G |
| S0013 | 25 | 248 | C | C | C | C | T | C | C | C |
| S0347 | 26 | 92 | C | C | G | C | G | C | G | C |
| S0330 | 27 | 743 | A | A | A | A | G | A | A | A |
| S0336 | 28 | 552 | C | C | T | C | T | T | C | T |

| | SNP Detection | | | | | | | | |
|---|---|---|---|---|---|---|---|---|---|
| Marker | Yumeakari | Hanaechizen | Koshihikari | Tsukinohikari | Akitakomachi | Asanohikari | Aichinokaori | Matsuribare | Hinohikari |
| name | yma | hez | ksh | tkh | akk | ash | ank | mtb | hnh |
| S0015 | T | C | T | T | T | C | C | T | C |
| S0040 | A | A | A | T | A | T | T | T | A |
| S0279 | T | T | T | C | T | T | T | T | T |
| S0044 | T | T | C | T | T | T | T | T | T |
| S0252 | C | C | T | T | T | T | C | T | T |
| S0109 | C | T | C | T | C | T | C | T | C |
| S0115 | T | G | G | T | G | T | G | T | G |
| S0107 | G | G | G | G | G | G | G | G | G |
| S0126 | G | T | T | T | T | T | T | T | T |
| S0124 | G | G | G | G | G | G | G | G | G |
| S0146 | A | C | A | A | A | A | C | A | C |
| S0135 | G | G | T | G | G | G | G | G | G |
| S0155 | T | G | G | T | T | G | G | T | T |
| S0161 | G | A | A | A | G | A | G | A | A |
| S0177 | A | G | A | G | A | A | A | A | A |
| S0178 | C | C | T | T | C | C | C | C | C |
| S0174 | A | A | A | G | A | G | G | G | A |
| S0185 | C | C | C | C | C | C | C | C | C |
| S0208 | G | C | G | C | G | C | G | G | G |
| S0007 | A | A | A | A | A | A | A | A | A |
| S0070 | A | G | A | A | A | A | A | A | A |
| S0310 | C | T | C | T | C | T | T | T | T |
| S0375 | G | G | G | C | G | C | G | C | C |
| S0346 | G | G | A | A | G | G | G | G | G |
| S0013 | C | C | C | C | C | C | C | C | C |
| S0347 | G | G | G | G | G | C | G | G | C |
| S0330 | A | A | A | A | A | A | A | A | A |
| S0336 | T | T | T | C | T | C | C | C | T |

| | SNP Detection | | | | | | | | |
|---|---|---|---|---|---|---|---|---|---|
| Marker | Yumetsukushi | Hitomebore | Manamusume | Fusaotome | Dontokoi | Kinuhikari | Sasanishiki | Akebono | Goropikari |
| name | ymt | hit | mmm | fom | don | knh | ssk | akb | grp |
| S0015 | C | T | T | T | T | C | T | C | T |
| S0040 | T | A | A | A | T | T | A | T | A |
| S0279 | T | T | T | T | T | T | T | T | T |
| S0044 | C | T | T | T | C | C | T | T | T |
| S0252 | T | T | C | C | C | T | T | C | T |
| S0109 | C | C | C | C | C | C | T | T | T |
| S0115 | G | G | G | G | T | G | T | T | G |
| S0107 | G | G | G | G | G | G | G | A | G |
| S0126 | T | T | T | T | T | T | G | T | T |
| S0124 | G | G | G | G | G | G | G | G | G |
| S0146 | A | C | C | C | A | A | C | C | C |
| S0135 | T | T | T | T | T | T | G | G | G |
| S0155 | G | G | G | G | T | G | G | G | T |
| S0161 | A | A | A | A | A | A | A | G | A |
| S0177 | A | A | A | A | A | A | A | A | G |
| S0178 | T | T | T | T | T | T | C | C | T |
| S0174 | A | A | A | A | A | A | G | G | G |
| S0185 | A | C | C | C | A | A | C | C | C |
| S0208 | G | G | G | C | G | G | G | G | C |
| S0007 | A | A | A | A | A | A | A | G | A |
| S0070 | A | A | A | A | G | A | A | G | A |
| S0310 | C | C | C | C | C | C | T | T | C |
| S0375 | G | C | C | G | C | G | C | C | C |
| S0346 | A | A | A | A | A | A | G | G | A |
| S0013 | C | C | C | C | C | C | C | T | C |
| S0347 | G | G | G | G | G | G | G | C | G |
| S0330 | A | A | A | A | A | A | A | A | A |
| S0336 | C | T | T | T | C | C | T | C | T |

Herein, the variety of a test rice can be identified by determining the nucleotide sequence at an above position (1) to (28) in its genome, and comparing that with data on the nucleotide sequences of the rice varieties, shown in Table 1. In a preferred embodiment of this invention, a nucleotide sequence is distinguished by using a polymorphic marker described in the above (1′) to (28′). In the methods of this invention, it is not necessary to determine the nucleotide sequences at all positions described in (1) to (28) above. For example, the polymorphic marker “S0124” may be used to determine the nucleotide sequence at position 323 in the nucleotide sequence of SEQ ID NO: 10, as shown in (10), above. If the nucleotide sequence is determined to be A (adenine), the test rice is identified as Kirara 397. In another case, the polymorphic markers “S0126” and “S0015” may be used in combination to determine the nucleotide sequences. If G is the nucleotide at position 475 in the nucleotide sequence of SEQ ID NO: 9, in (9) above; and C is the nucleotide at position 593 in the nucleotide sequence of SEQ ID NO: 1, in (1) above, then the test rice is identified as Yukinosei. Thus, using the nucleotide sequences determined in the genome of a test rice at positions described in the above (1) to (28), one skilled in the art can easily determine the rice variety based on Table 1, provided herein.

Furthermore, in the methods of this invention, it is not necessary to determine the type of nucleotide at the positions described in (1) to (28), above. Rice varieties may be distinguished by examining whether a nucleotide sequence, at an above position (1) to (28) in the genome of a test rice, is identical to a Nipponbare sequence at the same position. In a preferred embodiment of this invention, the polymorphic markers of the above (1′) to (28′) may be used to determine whether the nucleotide sequence in a test rice genome at a position (1) to (28) above, is identical to a Nipponbare sequence at this position.

For each of the above rice varieties, the present inventors examined whether or not the nucleotide sequences at positions (1) to (28) above are identical to those of Nipponbare, and they established combinations of polymorphic markers that enable the above varieties to be distinguished (Tables 2 to 7). In Tables 2 to 7, the combinations of polymorphic markers that can distinguish varieties are shaded. Combinations of polymorphic markers are not limited to those shown in Tables 2 to 7, and those skilled in the art can appropriately select combinations of polymorphic markers that can be used to distinguish varieties, according to nucleotide sequence information at positions (1) to (28) above in the genome of 26 rice varieties, providedbythepresent invention. In the tables, a circle shows a match with Nipponbare, while a cross indicates a mismatch.

Table 2

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Nipponbare

| | | | | | | | | | |
|---|---|---|---|---|---|---|---|---|---|
| S0107 | 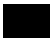 | ◯ | X | X | X | X | X | X | X |
| S0177 | 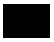 | X | X | X | X | X | X | X | X |

Hatsushimo

| | | | | | | | | | |
|---|---|---|---|---|---|---|---|---|---|
| S0107 | ◯ | 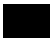 | X | X | X | X | X | X | X |
| S0177 | ◯ | 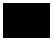 | X | X | X | X | X | X | X |
| S0185 | ◯ | 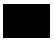 | ◯ | ◯ | ◯ | X | X | X | X |

Mutsuhomare

| | | | | | | | | | |
|---|---|---|---|---|---|---|---|---|---|
| S0070 | ◯ | ◯ | 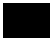 | ◯ | ◯ | ◯ | X | ◯ | ◯ |
| S0161 | ◯ | X | 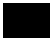 | ◯ | ◯ | X | ◯ | ◯ | X |
| S0109 | ◯ | ◯ | 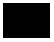 | ◯ | X | X | X | X | X |

Yukinosei

| | | | | | | | | | |
|---|---|---|---|---|---|---|---|---|---|
| S0126 | ◯ | ◯ | ◯ | 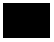 | ◯ | ◯ | ◯ | ◯ | X |
| S0015 | ◯ | ◯ | ◯ | 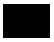 | X | X | ◯ | X | X |

Kirara 397

| | | | | | | | | | |
|---|---|---|---|---|---|---|---|---|---|
| S0124 | ◯ | ◯ | ◯ | ◯ | 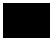 | ◯ | ◯ | ◯ | ◯ |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Nipponbare

| | | | | | | | | | |
|---|---|---|---|---|---|---|---|---|---|
| S0107 | X | X | X | X | X | X | X | X | X |
| S0177 | ◯ | X | ◯ | X | X | X | X | X | X |

Hatsushimo

| | | | | | | | | | |
|---|---|---|---|---|---|---|---|---|---|
| S0107 | X | X | X | X | X | X | X | X | X |
| S0177 | ◯ | X | ◯ | X | X | X | X | X | X |
| S0185 | X | X | X | X | X | X | X | X | ◯ |

Mutsuhomare

| | | | | | | | | | |
|---|---|---|---|---|---|---|---|---|---|
| S0070 | X | ◯ | ◯ | ◯ | ◯ | ◯ | ◯ | ◯ | ◯ |
| S0161 | ◯ | ◯ | ◯ | X | ◯ | X | ◯ | ◯ | ◯ |
| S0109 | ◯ | X | ◯ | X | ◯ | X | ◯ | X | X |

Yukinosei

| | | | | | | | | | |
|---|---|---|---|---|---|---|---|---|---|
| S0126 | ◯ | ◯ | ◯ | ◯ | ◯ | ◯ | ◯ | ◯ | ◯ |
| S0015 | ◯ | X | X | X | ◯ | ◯ | X | ◯ | ◯ |

Kirara 397

| | | | | | | | | | |
|---|---|---|---|---|---|---|---|---|---|
| S0124 | ◯ | ◯ | ◯ | ◯ | ◯ | ◯ | ◯ | ◯ | ◯ |

| Marker | Hitomebore | Manamusume | Fusaotome | Dontokoi | Kinuhikari | Sasanishiki | Akebono | Goropikari |
|---|---|---|---|---|---|---|---|---|
| Nipponbare | | | | | | | | |
| S0107 | X | X | X | X | X | X | ◯ | X |
| S0177 | X | X | X | X | X | X | X | ◯ |
| Hatsushimo | | | | | | | | |
| S0107 | X | X | X | X | X | X | ◯ | X |
| S0177 | X | X | X | X | X | X | X | ◯ |
| S0185 | X | X | X | ◯ | ◯ | X | X | X |
| Mutsuhomare | | | | | | | | |
| S0070 | ◯ | ◯ | ◯ | X | ◯ | ◯ | X | ◯ |
| S0161 | ◯ | ◯ | ◯ | ◯ | ◯ | ◯ | X | ◯ |
| S0109 | X | X | X | X | X | ◯ | ◯ | ◯ |
| Yukinosei | | | | | | | | |
| S0126 | ◯ | ◯ | ◯ | ◯ | ◯ | X | ◯ | ◯ |
| S0015 | X | X | X | X | ◯ | X | ◯ | X |
| Kirara 397 | | | | | | | | |
| S0124 | ◯ | ◯ | ◯ | ◯ | ◯ | ◯ | ◯ | ◯ |

TABLE 3

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Tsugaruroman

| | | | | | | | | | |
|---|---|---|---|---|---|---|---|---|---|
| S0161 | ◯ | X | X | ◯ | ◯ | 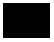 | ◯ | ◯ | X |
| S0252 | ◯ | X | X | X | X | 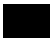 | ◯ | ◯ | X |
| S0115 | ◯ | X | X | X | X | 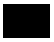 | ◯ | X | ◯ |

Gohyakumangoku

| | | | | | | | | | |
|---|---|---|---|---|---|---|---|---|---|
| S0135 | ◯ | ◯ | ◯ | ◯ | ◯ | ◯ | 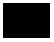 | ◯ | ◯ |
| S0174 | ◯ | ◯ | ◯ | ◯ | ◯ | X | 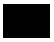 | X | X |

Morinokumasan

| | | | | | | | | | |
|---|---|---|---|---|---|---|---|---|---|
| S0178 | ◯ | ◯ | ◯ | ◯ | X | ◯ | X | 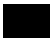 | ◯ |
| S0015 | ◯ | ◯ | ◯ | ◯ | X | X | ◯ | 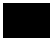 | X |
| S0161 | ◯ | X | X | ◯ | ◯ | X | ◯ | 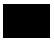 | X |
| S0109 | ◯ | ◯ | X | ◯ | X | X | X | 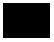 | X |

Yumeakari

| | | | | | | | | | |
|---|---|---|---|---|---|---|---|---|---|
| S0126 | ◯ | ◯ | ◯ | X | ◯ | ◯ | ◯ | ◯ | 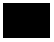 |
| S0109 | ◯ | ◯ | X | ◯ | X | X | X | X | 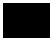 |

Hanaechizen

| | | | | | | | | | |
|---|---|---|---|---|---|---|---|---|---|
| S0177 | ◯ | X | X | X | X | X | X | X | X |
| S0174 | ◯ | ◯ | ◯ | ◯ | ◯ | X | ◯ | X | X |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Tsugaruroman

| | | | | | | | | | |
|---|---|---|---|---|---|---|---|---|---|
| S0161 | ◯ | ◯ | ◯ | X | ◯ | X | ◯ | ◯ | ◯ |
| S0252 | X | ◯ | ◯ | ◯ | ◯ | X | ◯ | ◯ | ◯ |
| S0115 | X | X | ◯ | X | ◯ | X | ◯ | X | X |

Gohyakumangoku

| | | | | | | | | | |
|---|---|---|---|---|---|---|---|---|---|
| S0135 | ◯ | X | ◯ | ◯ | ◯ | ◯ | ◯ | ◯ | X |
| S0174 | X | X | ◯ | X | ◯ | ◯ | ◯ | X | X |

Morinokumasan

| | | | | | | | | | |
|---|---|---|---|---|---|---|---|---|---|
| S0178 | ◯ | X | X | ◯ | ◯ | ◯ | ◯ | ◯ | X |
| S0015 | ◯ | X | X | X | ◯ | ◯ | X | ◯ | ◯ |
| S0161 | ◯ | ◯ | ◯ | X | ◯ | X | ◯ | ◯ | ◯ |
| S0109 | ◯ | X | ◯ | X | ◯ | X | ◯ | X | X |

Yumeakari

| | | | | | | | | | |
|---|---|---|---|---|---|---|---|---|---|
| S0126 | ◯ | ◯ | ◯ | ◯ | ◯ | ◯ | ◯ | ◯ | ◯ |
| S0109 | ◯ | X | ◯ | X | ◯ | X | ◯ | X | X |

Hanaechizen

| | | | | | | | | | |
|---|---|---|---|---|---|---|---|---|---|
| S0177 | 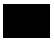 | X | ◯ | X | X | X | X | X | X |
| S0174 | 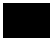 | X | ◯ | X | ◯ | ◯ | ◯ | X | X |

| Marker | Hitomebore | Manamusume | Fusaotome | Dontokoi | Kinuhikari | Sasanishiki | Akebono | Goropikari |
|---|---|---|---|---|---|---|---|---|
| Tsugaruroman | | | | | | | | |
| S0161 | ◯ | ◯ | ◯ | ◯ | ◯ | ◯ | X | ◯ |
| S0252 | ◯ | X | X | X | ◯ | ◯ | X | ◯ |
| S0115 | X | X | X | ◯ | X | ◯ | ◯ | X |
| Gohyakumangoku | | | | | | | | |
| S0135 | X | X | X | X | X | ◯ | ◯ | ◯ |
| S0174 | X | X | X | X | X | ◯ | ◯ | ◯ |
| Morinokumasan | | | | | | | | |
| S0178 | X | X | X | X | X | ◯ | ◯ | X |
| S0015 | X | X | X | X | ◯ | X | ◯ | X |
| S0161 | ◯ | ◯ | ◯ | ◯ | ◯ | ◯ | X | ◯ |
| S0109 | X | X | X | X | X | ◯ | ◯ | ◯ |
| Yumeakari | | | | | | | | |
| S0126 | ◯ | ◯ | ◯ | ◯ | ◯ | X | ◯ | ◯ |
| S0109 | X | X | X | X | X | ◯ | ◯ | ◯ |
| Hanaechizen | | | | | | | | |
| S0177 | X | X | X | X | X | X | X | ◯ |
| S0174 | X | X | X | X | X | ◯ | ◯ | ◯ |

TABLE 4

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Koshihikari

| | | | | | | | | | |
|---|---|---|---|---|---|---|---|---|---|
| S0040 | ◯ | ◯ | ◯ | X | X | X | X | X | X |
| S0044 | ◯ | ◯ | ◯ | ◯ | ◯ | ◯ | ◯ | ◯ | ◯ |

Tsukinohikari

| | | | | | | | | | |
|---|---|---|---|---|---|---|---|---|---|
| S0279 | ◯ | X | X | X | X | X | X | X | X |
| S0107 | ◯ | ◯ | X | X | X | X | X | X | X |

Akitakomachi

| | | | | | | | | | |
|---|---|---|---|---|---|---|---|---|---|
| S0115 | ◯ | X | X | X | X | ◯ | ◯ | X | ◯ |
| S0146 | ◯ | ◯ | ◯ | ◯ | X | X | ◯ | ◯ | X |
| S0178 | ◯ | ◯ | ◯ | ◯ | X | ◯ | X | ◯ | ◯ |

Asanohikari

| | | | | | | | | | |
|---|---|---|---|---|---|---|---|---|---|
| S0208 | ◯ | X | X | X | X | X | X | X | X |
| S0146 | ◯ | ◯ | ◯ | ◯ | X | X | ◯ | ◯ | X |
| S0177 | ◯ | X | X | X | X | X | X | X | X |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Koshihikari

| | | | | | | | | | |
|---|---|---|---|---|---|---|---|---|---|
| S0040 | X | 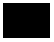 | ◯ | X | ◯ | ◯ | ◯ | X | ◯ |
| S0044 | ◯ | 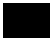 | ◯ | ◯ | ◯ | ◯ | ◯ | ◯ | X |

Tsukinohikari

| | | | | | | | | | |
|---|---|---|---|---|---|---|---|---|---|
| S0279 | X | X | 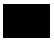 | X | X | X | X | X | X |
| S0107 | X | X | 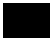 | X | X | X | X | X | X |

Akitakomachi

| | | | | | | | | | |
|---|---|---|---|---|---|---|---|---|---|
| S0115 | X | X | ◯ | 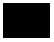 | ◯ | X | ◯ | X | X |
| S0146 | ◯ | X | X | 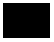 | X | ◯ | X | ◯ | X |
| S0178 | ◯ | X | X | 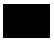 | ◯ | ◯ | ◯ | ◯ | X |

Asanohikari

| | | | | | | | | | |
|---|---|---|---|---|---|---|---|---|---|
| S0208 | ◯ | X | ◯ | X | 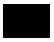 | X | X | X | X |
| S0146 | ◯ | X | X | X | 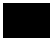 | ◯ | X | ◯ | X |
| S0177 | ◯ | X | ◯ | X | 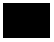 | X | X | X | X |

| Marker | Hitomebore | Manamusume | Fusaotome | Dontokoi | Kinuhikari | Sasanishiki | Akebono | Goropikari |
|---|---|---|---|---|---|---|---|---|
| Koshihikari | | | | | | | | |
| S0040 | X | X | X | ◯ | ◯ | X | ◯ | X |
| S0044 | ◯ | ◯ | ◯ | X | X | ◯ | ◯ | ◯ |
| Tsukinohikari | | | | | | | | |
| S0279 | X | X | X | X | X | X | X | X |
| S0107 | X | X | X | X | X | X | ◯ | X |
| Akitakomachi | | | | | | | | |
| S0115 | X | X | X | ◯ | X | ◯ | ◯ | X |
| S0146 | ◯ | ◯ | ◯ | X | X | ◯ | ◯ | ◯ |
| S0178 | X | X | X | X | X | ◯ | ◯ | X |

Asanohikari

| | | | | | | | | |
|---|---|---|---|---|---|---|---|---|
| S0208 | X | X | ◯ | X | X | X | X | ◯ |
| S0146 | ◯ | ◯ | ◯ | X | X | ◯ | ◯ | ◯ |
| S0177 | X | X | X | X | X | X | X | ◯ |

TABLE 5

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Aichinokaori

| | | | | | | | | | |
|---|---|---|---|---|---|---|---|---|---|
| S0109 | ◯ | ◯ | X | ◯ | X | X | X | X | X |
| S0155 | ◯ | ◯ | X | ◯ | X | X | X | X | X |
| S0104 | ◯ | ◯ | ◯ | ◯ | ◯ | X | ◯ | X | X |

Matsuribare

| | | | | | | | | | |
|---|---|---|---|---|---|---|---|---|---|
| S0109 | ◯ | ◯ | X | ◯ | X | X | X | X | X |
| S0208 | ◯ | X | X | X | X | X | X | X | X |
| S0146 | ◯ | ◯ | ◯ | ◯ | X | X | ◯ | ◯ | X |

Hinohikari

| | | | | | | | | | |
|---|---|---|---|---|---|---|---|---|---|
| S0015 | ◯ | ◯ | ◯ | ◯ | X | X | ◯ | X | X |
| S0155 | ◯ | ◯ | X | ◯ | X | X | X | X | X |
| S0174 | ◯ | ◯ | ◯ | ◯ | ◯ | X | ◯ | X | X |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Aichinokaori

| | | | | | | | | | |
|---|---|---|---|---|---|---|---|---|---|
| S0109 | ◯ | X | ◯ | X | ◯ | 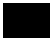 | ◯ | X | X |
| S0155 | ◯ | ◯ | X | X | ◯ | 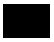 | X | X | ◯ |
| S0104 | X | X | ◯ | X | ◯ | 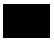 | ◯ | X | X |

Matsuribare

| | | | | | | | | | |
|---|---|---|---|---|---|---|---|---|---|
| S0109 | ◯ | X | ◯ | X | ◯ | X | 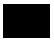 | X | X |
| S0208 | ◯ | X | ◯ | X | ◯ | X | 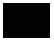 | X | X |
| S0146 | ◯ | X | X | X | X | ◯ | 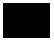 | ◯ | X |

Hinohikari

| | | | | | | | | | |
|---|---|---|---|---|---|---|---|---|---|
| S0015 | ◯ | X | X | X | ◯ | ◯ | X | 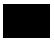 | ◯ |
| S0155 | ◯ | ◯ | X | X | ◯ | ◯ | X | 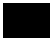 | ◯ |
| S0174 | X | X | ◯ | X | ◯ | ◯ | ◯ | 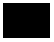 | X |

| Marker | Hitomebore | Manamusume | Fusaotome | Dontokoi | Kinuhikari | Sasanishiki | Akebono | Goropikari |
|---|---|---|---|---|---|---|---|---|
| Aichinokaori | | | | | | | | |
| S0109 | X | X | X | X | X | ◯ | ◯ | ◯ |
| S0155 | ◯ | ◯ | ◯ | X | ◯ | ◯ | ◯ | X |
| S0104 | X | X | X | X | X | ◯ | ◯ | ◯ |

| Matsuribare | | | | | | | | |
|---|---|---|---|---|---|---|---|---|
| S0109 | X | X | X | X | X | ◯ | ◯ | ◯ |
| S0208 | ◯ | ◯ | ◯ | X | ◯ | ◯ | ◯ | X |
| S0146 | ◯ | ◯ | ◯ | X | X | ◯ | ◯ | ◯ |

| Hinohikari | | | | | | | | |
|---|---|---|---|---|---|---|---|---|
| S0015 | X | X | X | X | ◯ | X | ◯ | X |
| S0155 | ◯ | ◯ | ◯ | X | ◯ | ◯ | ◯ | X |
| S0174 | X | X | X | X | X | ◯ | ◯ | ◯ |

TABLE 6

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Hitomebore

| | | | | | | | | | |
|---|---|---|---|---|---|---|---|---|---|
| S0044 | ◯ | ◯ | ◯ | ◯ | ◯ | ◯ | ◯ | ◯ | ◯ |
| S0135 | ◯ | ◯ | ◯ | ◯ | ◯ | ◯ | X | ◯ | ◯ |
| S0115 | ◯ | X | X | X | X | ◯ | ◯ | X | ◯ |
| S0252 | ◯ | X | X | X | X | ◯ | ◯ | ◯ | X |

Manamusume

| | | | | | | | | | |
|---|---|---|---|---|---|---|---|---|---|
| S0135 | ◯ | ◯ | ◯ | ◯ | ◯ | ◯ | X | ◯ | ◯ |
| S0208 | ◯ | X | X | X | X | X | X | X | X |
| S0252 | ◯ | X | X | X | X | ◯ | ◯ | ◯ | X |
| S0146 | ◯ | ◯ | ◯ | ◯ | X | X | ◯ | ◯ | X |

Fusaotome

| | | | | | | | | | |
|---|---|---|---|---|---|---|---|---|---|
| S0135 | ◯ | ◯ | ◯ | ◯ | ◯ | ◯ | X | ◯ | ◯ |
| S0208 | ◯ | X | X | X | X | X | X | X | X |

Dontokoi

| | | | | | | | | | |
|---|---|---|---|---|---|---|---|---|---|
| S0044 | ◯ | ◯ | ◯ | ◯ | ◯ | ◯ | ◯ | ◯ | ◯ |
| S0252 | ◯ | X | X | X | X | ◯ | ◯ | ◯ | X |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Hitomebore

| | | | | | | | | | |
|---|---|---|---|---|---|---|---|---|---|
| S0044 | ◯ | X | ◯ | ◯ | ◯ | ◯ | ◯ | ◯ | X |
| S0135 | ◯ | X | ◯ | ◯ | ◯ | ◯ | ◯ | ◯ | X |
| S0115 | X | X | ◯ | X | ◯ | X | ◯ | X | X |
| S0252 | X | ◯ | ◯ | ◯ | ◯ | X | ◯ | ◯ | ◯ |

Manamusume

| | | | | | | | | | |
|---|---|---|---|---|---|---|---|---|---|
| S0135 | ◯ | X | ◯ | ◯ | ◯ | ◯ | ◯ | ◯ | X |
| S0208 | ◯ | X | ◯ | X | ◯ | X | X | X | X |
| S0252 | X | ◯ | ◯ | ◯ | ◯ | X | ◯ | ◯ | ◯ |
| S0146 | ◯ | X | X | X | X | ◯ | X | ◯ | X |

Fusaotome

| | | | | | | | | | |
|---|---|---|---|---|---|---|---|---|---|
| S0135 | ◯ | X | ◯ | ◯ | ◯ | ◯ | ◯ | ◯ | X |
| S0208 | ◯ | X | ◯ | X | ◯ | X | X | X | X |

Dontokoi

| | | | | | | | | | |
|---|---|---|---|---|---|---|---|---|---|
| S0044 | ◯ | X | ◯ | ◯ | ◯ | ◯ | ◯ | ◯ | X |
| S0252 | X | ◯ | ◯ | ◯ | ◯ | X | ◯ | ◯ | ◯ |

| Marker | Hitomebore | Manamusume | Fusaotome | Dontokoi | Kinuhikari | Sasanishiki | Akebono | Goropikari |
|---|---|---|---|---|---|---|---|---|
| Hitomebore | | | | | | | | |
| S0044 | 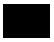 | ◯ | ◯ | X | X | ◯ | ◯ | ◯ |
| S0135 | 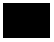 | X | X | X | X | ◯ | ◯ | ◯ |
| S0115 | 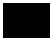 | X | X | ◯ | X | ◯ | ◯ | X |
| S0252 | 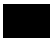 | X | X | X | ◯ | ◯ | X | ◯ |
| Manamusume | | | | | | | | |
| S0135 | X | 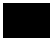 | X | X | X | ◯ | ◯ | ◯ |
| S0208 | X | 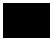 | ◯ | X | X | X | X | ◯ |
| S0252 | ◯ | 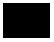 | X | X | ◯ | ◯ | X | ◯ |
| S0146 | ◯ | 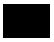 | ◯ | X | X | ◯ | ◯ | ◯ |
| Fusaotome | | | | | | | | |
| S0135 | X | X | 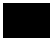 | X | X | ◯ | ◯ | ◯ |
| S0208 | X | X | 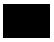 | X | X | X | X | ◯ |
| Dontokoi | | | | | | | | |
| S0044 | ◯ | ◯ | ◯ | 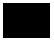 | X | ◯ | ◯ | ◯ |
| S0252 | ◯ | X | X | 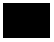 | ◯ | ◯ | X | ◯ |

TABLE 7

| | | | | | | | | | |
|---|---|---|---|---|---|---|---|---|---|
| Marker | Nipponbare | Hatsushimo | Mutsuhomare | Yukinosei | Kirara 397 | Tsugaruroman | Gohyakumangoku | Morinokumasan | Yumeakari |

Yumetsukushi - Kinuhikari

| | | | | | | | | | |
|---|---|---|---|---|---|---|---|---|---|
| S0044 | ◯ | ◯ | ◯ | ◯ | ◯ | ◯ | ◯ | ◯ | ◯ |
| S0015 | ◯ | ◯ | ◯ | ◯ | X | X | ◯ | X | X |

Sasanishiki

| | | | | | | | | | |
|---|---|---|---|---|---|---|---|---|---|
| S0126 | ◯ | ◯ | ◯ | X | ◯ | ◯ | ◯ | ◯ | X |
| S0252 | ◯ | X | X | X | X | ◯ | ◯ | ◯ | X |

Akebono

| | | | | | | | | | |
|---|---|---|---|---|---|---|---|---|---|
| S0161 | ◯ | X | X | ◯ | ◯ | X | ◯ | ◯ | X |
| S0007 | ◯ | ◯ | ◯ | X | X | ◯ | X | ◯ | ◯ |

Goropikari

| | | | | | | | | | |
|---|---|---|---|---|---|---|---|---|---|
| S0177 | ◯ | X | X | X | X | X | X | X | X |
| S0155 | ◯ | ◯ | X | ◯ | X | X | X | X | X |
| S0115 | ◯ | X | X | X | X | ◯ | ◯ | X | ◯ |

| | | | | | | | | | |
|---|---|---|---|---|---|---|---|---|---|
| Marker | Hanaechizen | Koshihikari | Tsukinohikari | Akitakomachi | Asanohikari | Aichinokaori | Matsuribare | Hinohikari | Yumetsukushi |

Yumetsukushi - Kinuhikari

| | | | | | | | | | |
|---|---|---|---|---|---|---|---|---|---|
| S0044 | ◯ | X | ◯ | ◯ | ◯ | ◯ | ◯ | ◯ | 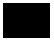 |
| S0015 | ◯ | X | X | X | ◯ | ◯ | X | ◯ | 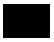 |

Sasanishiki

| | | | | | | | | | |
|---|---|---|---|---|---|---|---|---|---|
| S0126 | ◯ | ◯ | ◯ | ◯ | ◯ | ◯ | ◯ | ◯ | ◯ |
| S0252 | X | ◯ | ◯ | ◯ | ◯ | X | ◯ | ◯ | ◯ |

Akebono

| | | | | | | | | | |
|---|---|---|---|---|---|---|---|---|---|
| S0161 | ◯ | ◯ | ◯ | X | ◯ | X | ◯ | ◯ | ◯ |
| S0007 | ◯ | ◯ | ◯ | ◯ | ◯ | ◯ | ◯ | ◯ | ◯ |

Goropikari

| | | | | | | | | | |
|---|---|---|---|---|---|---|---|---|---|
| S0177 | ◯ | X | ◯ | X | X | X | X | X | X |
| S0155 | ◯ | ◯ | X | X | ◯ | ◯ | X | X | ◯ |
| S0115 | X | X | ◯ | X | ◯ | X | ◯ | X | X |

| Marker | Hitomebore | Manamusume | Fusaotome | Dontokoi | Kinuhikari | Sasanishiki | Akebono | Goropikari |
|---|---|---|---|---|---|---|---|---|
| Yumetsukushi - Kinuhikari | | | | | | | | |
| S0044 | ◯ | ◯ | ◯ | X | 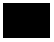 | ◯ | ◯ | ◯ |
| S0015 | X | X | X | X | 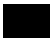 | X | ◯ | X |
| Sasanishiki | | | | | | | | |
| S0126 | ◯ | ◯ | ◯ | ◯ | ◯ | 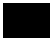 | ◯ | ◯ |
| S0252 | ◯ | X | X | X | ◯ | 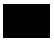 | X | ◯ |

Akebono

| S0161 | ◯ | ◯ | ◯ | ◯ | ◯ | ◯ | 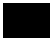 | ◯ |
|---|---|---|---|---|---|---|---|---|
| S0007 | ◯ | ◯ | ◯ | ◯ | ◯ | ◯ | 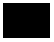 | ◯ |
| Goropikari | | | | | | | | |
| S0177 | X | X | X | X | X | X | X | 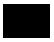 |
| S0155 | ◯ | ◯ | ◯ | X | ◯ | ◯ | ◯ | 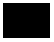 |
| S0115 | X | X | X | ◯ | X | ◯ | ◯ | 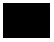 |

For example, a test rice may be examined using the polymorphic marker “S0135” for the nucleotide sequence at position 765 in the nucleotide sequence of SEQ ID NO: 12, as shown in the above (12), and “S0208” for the nucleotide sequence at position 178 of SEQ ID NO: 19, as shown in (19). If the nucleotide sequence of a test rice does not match with that of Nipponbare at the former site, but matches at the latter site, then the test rice is identified as Fusaotome. By determining the nucleotide sequences at the above positions using each of the polymorphic markers (1′) to (28′) above, the nucleotide sequence of a test rice and Nipponbare at a position (1) to (28) above can be found to match, or not to match, and the variety of a test rice can be easily distinguished based on Tables 2 to 7.

One skilled in the art can determine the nucleotide species in the above step (A) of this invention using a publicly known method for determining nucleotide sequences, a method for detecting polymorphic mutations, or the like. For example, in a preferred embodiment of this invention, the following method may be used: First, DNAs are prepared from a test rice. Herein, a test rice includes its leaf, root, seed, callus, leaf sheath, cultured cell, and the like, but it is not limited thereto. Furthermore, one skilled in the art may prepare DNAs from chromosomal DNAs extracted from the test rice. For example, in a preferred method, rice seeds may be disrupted in an alkaline aqueous solution, and then genomic DNAs may be extracted from the disrupted seeds; however, the methods are not limited thereto. In the above, the seeds are preferably polished.

In these methods, a DNA comprising a nucleotide at a position described in any of the above (1) to (28), or a nucleotide composing a base pair with the above nucleotide at a position on the complementary strand, is then amplified. Herein, a method for amplifying a DNA may be PCR, but is not limited thereto, as long as it enables DNA amplification.

In this method, the nucleotide sequence of an amplified DNA is then determined. Nucleotide sequences can be determined by methods commonly known to those skilled in the art.

In these methods, a determined nucleotide sequence is then compared with that of a control. Herein, the control is normally Nipponbare, which is represented by the sequences described in SEQ ID NO: 1 to NO: 28. Alternatively, one skilled in the art may obtain the nucleotide sequence information of a wild type Nipponbare genome from a variety of gene databases, references, or the like. In this method, polymorphisms are determined to be present or absent in a test rice genome by comparison with a control.

The methods for distinguishing rice varieties of this invention may be performed by a variety of methods for enabling polymorphism detection, instead of by directly determining the nucleotide sequence of a DNA derived from a test rice, as described above. For example, the methods of this invention for distinguishing rice varieties may be performed using the following methods:

First, DNAs are prepared from a test rice. Then, the prepared DNA is digested with a restriction enzyme. The DNA fragments are then fractionated by size, and the sizes of the detected fragments are compared with a control. In an alternative embodiment, DNAs are first prepared from a test rice. Then, DNA comprising a nucleotide described in any of the above (1) to (28), or a nucleotide composing a base pair with the above nucleotide in the complementary strand, is amplified. Then, the amplified DNA is digested with a restriction enzyme. The DNA fragments are then fractionated by size, and the sizes of the detected DNA fragments are compared with a control.

Examples of such methods are methods utilizing RFLP (restriction fragment length polymorphism), PCR-RFLP, or the like. Specifically, if a mutation exists in a restriction enzyme recognition site, or if a DNA fragment generated by restriction enzyme treatment comprises a base insertion or deletion, then the size of the fragment that results from restriction enzyme treatment should be different from that of a control. A region comprising such a mutation may be amplified by PCR, and treated with the corresponding restriction enzyme to detect the mutation by differences in band mobility after electrophoresis. Alternatively, chromosomal DNA may be treated with such restriction enzymes, separated by electrophoresis, and then Southern blotting may be performed using an oligonucleotide of this invention to detect the presence or absence of a mutation. One skilled in the art can appropriately choose restriction enzymes according to the mutations.

Furthermore, in another method, DNAs are first prepared from a test rice. Then, a DNA comprising a nucleotide described in any of the above (1) to (28), or a nucleotide composing a base pair with the above nucleotide on the complementary strand, is amplified. The amplified DNA is then dissociated into single strands. Dissociated single-strand DNA is separated on a non-denaturing gel. The mobility of the separated single-strand DNA on the gel is compared with that of a control.

An example of the above method is PCR-SSCP (single-strand conformation polymorphism) (Cloning and polymerase chain reaction-single-strand conformation polymorphism analysis of anonymous Alu repeats on chromosome 11. Genomics 12(1): 139-146 (1992, Jan. 1); Detection of p53 gene mutations in human brain tumors by single-strand conformation polymorphism analysis of polymerase chain reaction products. Oncogene 6(8): 1313-1318 (1991, Aug. 1); Multiple fluorescence-based PCR-SSCP analysis with postlabeling., PCR Methods Appl. 4(5): 275-282 (1995, Apr. 1)). This method is particularly suitable for screening a large number of DNA samples, due to advantages such as the relative ease of manipulation and the small amount of sample required. The principle of the method is as follows: When a double-stranded DNA fragment is dissociated into single strands, each strand gives rise to a unique conformation, according to its nucleotide sequence. Thus, when the dissociated DNA strands are separated by electrophoresis on a non-denaturing polyacrylamide gel, complementary single-stranded DNAs of the same length migrate to different positions, according to the differences in their respective conformations. The substitution of a single nucleotide can change the conformation of a single-stranded DNA, resulting in a different mobility during electrophoresis on a polyacrylamide gel. Thus, the presence of a mutation in a DNA fragment, such as a point mutation, deletion, and insertion, can be detected by detecting the mobility shift.

Specifically, a DNA comprising a nucleotide described in any of the above (1) to (28), or a nucleotide composing a base pair with the above nucleotide on the complementary strand, is first amplified by PCR or the like. Normally, a region for amplification is preferably about 200 bp to 400 bp long. One skilled in the art can perform PCR by appropriately selecting reaction conditions and the like. An amplified DNA product may be labeled during PCR using a primer labeled with an isotope such as ³²P, or a fluorescent dye, biotin, or the like. Alternatively, an amplified DNA product may be labeled by performing PCR in which nucleotide substrates labeled with an isotope such as ³²P, or a fluorescent dye, biotin, or the like have been added to the PCR mixture. Furthermore, amplified DNA fragments may be labeled after PCR by using Klenow enzyme or the like to attach nucleotide substrates labeled with an isotope such as ³²P, or a fluorescent dye, biotin, or the like. The resulting labeled DNA fragment is denatured by heating, for example, and subjected to electrophoresis on a polyacrylamide gel, without a denaturing agent such as urea. Conditions for separating DNA fragments may be improved by adding an appropriate amount of glycerol (about 5 to 10%) to the polyacrylamide gel. Conditions for electrophoresis may differ according to the properties of each DNA fragment; normally electrophoresis is performed at room temperature (20 to 25° C.). If a desired separation is not achieved, temperatures between 4 and 30° C. may be tested for optimal mobility. Following electrophoresis, the mobilities of DNA fragments are detected and analyzed by autoradiography using X-ray film, or by scanning on a scanner for fluorescence detection, or the like. When a band with different mobility is detected, it is directly cut from the gel, re-amplified using PCR, and subjected to direct sequencing to confirm the presence of a mutation. In addition, if not using labeled DNAs, the bands may be detected by staining the gels after electrophoresis with ethidium bromide, by silver staining, or the like.

Furthermore, in another method, DNAs are first prepared from a test rice (step (a)). Next, two different probes, oligonucleotides complementary to a nucleotide sequence near a DNA comprising a nucleotide sequence described in any of the above (1) to (28), or near to a nucleotide composing a base pair with the above nucleotide in the complementary strand, which are labeled with a fluorescent reporter and fluorescence quencher, are synthesized (step (b)). Then (step (c)), the DNA prepared in step (a) is hybridized with the probes synthesized in step (b). A DNA comprising a nucleotide described in any of the above (1) to (28), or a nucleotide composing a base pair with the above nucleotide in the complementary strand, is then amplified (step (d)), and reporter fluorescence emission is detected (step (e)). Then (step (f)), the fluorescent reporter emission detected in step (e) is compared with that of a control.

An example of such methods is TaqMan PCR (Strategies in SNP gene polymorphism. Kennichi Matsubara and Yoshiyuki Sakaki. Nakayama-Shoten p94-105; Genet. Anal. 14: 143-149 (1999)). Specifically, the 5′-end of probe is first labeled with a fluorescent reporter. Herein, fluorescent reporters include FAM and VIC, but are not limited thereto. In addition, the 3′-end of the above probe is labeled with a fluorescence quencher. Herein, a fluorescence quenchers may be any substance that can quench a fluorescent reporter. Then, probes labeled with the fluorescent reporter and fluorescence quencher are hybridized with the prepared DNA. Hybridization is normally performed under stringent conditions. Stringent conditions are, for example, normally 42° C., 2×SSC and 0.1% SDS, preferably 50° C., 2×SSC and 0.1% SDS, and more preferably 65° C., 0.1×SSC and 0.1% SDS, but are not limited thereto. A number of factors such as temperature and salt concentration can affect hybridization stringency, and one skilled in the art can achieve optimal stringencies by appropriately selecting the above factors.

A DNA comprising a nucleotide sequence described in any of the above (1) to (28), or a nucleotide composing a base pair with the above nucleotide in the complementary strand, is amplified using a DNA polymerase comprising 5′-nuclease activity. As a result, the fluorescent reporter-labeled moiety of the probe labeled with the fluorescent reporter and fluorescence quencher is digested, and the fluorescent reporter is released. Herein, the DNA polymerase with 5′-nuclease activity is preferably Taq DNA polymerase, but is not limited thereto. In this method, the released fluorescent reporter is then detected, and the fluorescent reporter emission is compared with that of a control.

Furthermore, in another method, DNAs are first prepared from a test rice (step (a)). Then, a probe is synthesized in which a sequence complementary to the 3′-flanking nucleotide sequence comprising a nucleotide sequence described in any of the above (1) to (28), or a nucleotide composing a base pair with the above nucleotide in the complementary strand, is combined with an unrelated sequence (step (b)). Then another probe, complementary at the 5′-flanking region of the nucleotide sequence described in any of the above (1) to (28), or the nucleotide composing a base pair with the above nucleotide in the complementary strand, is synthesized (step (c)). Then (step (d)), the probe synthesized in step (c) is hybridized with the DNA prepared in step (a). The DNA hybridized in step (d) is then digested with a single strand DNA cleavage enzyme, liberating part of the probe synthesized in step (b) (step (e)). Herein, the single strand DNA cleavage enzyme is not specifically limited; for example, cleavase can be used as described below. In this method, the probe liberated in step (e) is then hybridized with a probe for detection (step (f)). Then (step (g)), the DNA hybridized in step (f) is enzymatically digested, and the intensity of fluorescence thus emitted is measured (step (g)). Then (step (h)), the fluorescence intensity measured in step (g) is compared with that of a control.

An example of the above method is the Invader method (Strategies in SNP gene polymorphism. Kennichi Matsubara and Yoshiyuki Sakaki. Nakayama-Shoten p 94-105; Genome Research 10: 330-343 (2000)). Specifically, a probe complementary to a template in the 3′-flanking region of a nucleotide sequence described in any of the above (1) to (28), or a nucleotide composing a base pair with the above nucleotide in the complementary strand, and comprising a sequence unrelated to the template (flap) in the 5′-flanking region, is first synthesized (probe A). Next, a probe comprising a sequence complementary to the template in the 5′-flanking region of the nucleotide sequence described in any of the above (1) to (28), or the nucleotide composing a base pair with the above nucleotides in the complementary strand, is synthesized (probe B). In probe B, the nucleotide corresponding to a nucleotide described in any of the above (1) to (28), or the nucleotide composing a base pair with the above nucleotide in the complementary strand, may be any species. Then, the probes are hybridized with the prepared template DNA. The nucleotide in probe B that corresponds to the nucleotide sequence described in any of the above (1) to (28), or the nucleotide composing a base pair with the above nucleotide in the complementary strand, then creates a region comprising a flap at the 5′-end as a result of invasion. Thus, the hybridized DNA is digested with an endonuclease (cleavase) that recognizes the region comprising the flap, and cuts probe A at the 3′-side of the corresponding nucleotide. As a result, the flap is released. The released flap is then hybridized with a detection probe. The detection probe is generally called a fluorescence resonance energy transfer (FRET) probe. The probe has a 5′-region in which it can form complementary binding, and the 3′-region is complementary to the flap. Within the 5′-region, which can associate with itself complementarily, the 5′-end and 3′-side thereof are respectively labeled with the fluorescent reporter and fluorescence quencher. Upon hybridization, the nucleotides in the 3′-end of the released flap invade into the complementary binding sites of the FRET probe labeled with the fluorescent reporter, and create a structure that is recognized by cleavase. Herein, the fluorescent reporter released by digestion of the region labeled with the fluorescent reporter using cleavase is detected, and the intensity of the measured fluorescence is compared with that of a control.

Furthermore, in another method, DNAs are first prepared from a test rice (step (a)). Then, a DNA comprising a nucleotide as described in any of the above (1) to (28), or a nucleotide composing a base pair with the above nucleotide in the complementary strand, is amplified (step (b)). The amplified DNA is then dissociated into single-strands (step (c)). Next (step (d)), only one strand of the dissociated single-strand DNAs is isolated. A single nucleotide elongation reaction is then performed from close to a nucleotide described in any of the above (1) to (28), or a nucleotide composing a base pair with the above nucleotide in the complementary strand; the pyrophosphate thus generated is enzymatically illuminated; and the intensity of this illumination is measured (step (e)). Then, the fluorescence intensity measured in step (e) is compared with that of a control (step (f)). An example of such a method is the Pyrosequencing method (Anal. Biochem. 10: 103-110 (2000)).

Furthermore, in another method, DNAs are first prepared from a test rice (step (a)). Then, a DNA comprising a nucleotide described in any of the above (1) to (28), or a nucleotide composing a base pair with the above nucleotide in the complementary strand, is amplified (step (b)). A primer is then synthesized that is complementary to the sequence up until the nucleotide next to the nucleotide described in any of the above (1) to (28), or the nucleotide composing a base pair with the above nucleotide in the complementary strand (step (c)). Then (step (d)), a single nucleotide extension reaction is carried out in the presence of fluorescently labeled nucleotides, using the DNA amplified in step (b) as a template, with the primers synthesized in step (c). Then, fluorescence polarization is measured (step (e)). The fluorescence polarization measured in step (e) is then compared with that of a control (step (f)). An example of such a method is the AcycloPrime method (Genome Research 9: 492-498 (1999)).

The AcycloPrime method uses a pair of primers for amplifying the genome, and a single primer for detecting SNPs. First, a genomic region containing SNPs is amplified by PCR. This step is performed as in standard genomic PCR. Next, the amplified PCR product is annealed with a primer for detecting polymorphisms, and an elongation reaction is carried out. The primer for detecting polymorphisms is designed to anneal to a region adjacent to the target polymorphic site. Normally, the nucleotide substrate for the elongation reaction is nucleotide derivatives labeled with a fluorescent dye with its 3′-OH blocked (terminator). Therefore, the elongation is stopped on incorporation of a single nucleotide complementary to a nucleotide at a polymorphic site. Incorporation of the nucleotide derivative into the primer can be detected as an increase in fluorescence polarization (FP), due to an increase in molecular weight. By labeling using two different FP dyes that comprise distinct wavelengths, a nucleotide at a particular polymorphic site can be distinguished between two nucleotides. Because the level of fluorescence polarization can be quantified, it is possible to determine whether a target allele is homogenous or heterogeneous by running a single analysis. The above step (A) in the methods of the present invention may be preferably performed by the AcycloPrime method.

One skilled in the art can appropriately prepare the primers for genomic amplification and primers for detecting polymorphisms used in the AcycloPrime method, according to the information on genomic sequence and polymorphic sites. Examples of the primers for genomic amplification and primers for detecting polymorphisms, used in the methods of this invention to distinguish rice varieties using the AcycloPrime method, are shown in Tables 8 and 9, but they are not limited thereto.

TABLE 8

| | | | | | | | Polymorphism | | | | | | | |
|---|---|---|---|---|---|---|---|---|---|---|---|---|---|---|
| | | Genomic | | | | | (SNP) | | | | | | | |
| | | PCR | | | | | detection | | | | | | | |
| | | | | | | | | | Primer | | | | | |
| | | | | SEQ | | | SEQ | | nucleotide | | SEQ | | | |
| | | Marker | Primer nucleotide | ID | | | ID | Posi- | sequence | | ID | Termi- | | |
| chr | cM | name | sequence (5′-3′) | NO: | mer | Size | NO: | tion | (5′-3′) | mer | NO: | nator | SNPs | |
| 1 | 96.1 | S0015 | GCA ATT CCC ACT GGA AGA AT | 29 | 20 | 803 | 1 | 593 | AGG TCG ACA CTT CGG | 20 | 85 | C/T | Nipponbare | Others |
| | | | TAA GTT GGG GAA TGC GAT GT | 30 | 20 | bp | | | CCG TT | | | | C | T |
| 3 | 20.3 | S0040 | TCT GCT GCC TCT GCA CAT AC | 31 | 20 | 902 | 2 | 304 | GAA CAG CTG TAA TAA | 21 | 86 | A/T | Nipponbare | Others |
| | | | AAA AAC GAC ACC ACA TCA GCA | 32 | 21 | bp | | | GAC TGA | | | | T | A |
| 3 | 69.2 | S0279 | GGG GCG CTC CTT CAA AAC TT | 33 | 20 | 796 | 3 | 450 | GAT CCC TGC AAA GTC | 20 | 87 | C/T | Nipponbare | Others |
| | | | GGT TTG GCA CAC CAC AAT GG | 34 | 20 | bp | | | CCC AC | | | | C | T |
| 3 | 146.4 | S0044 | TGC AAT GTG CCA TTC CAT AG | 35 | 20 | 901 | 4 | 377 | CGC AAA CCA TCA ACT | 20 | 88 | C/T | Nipponbare | Others |
| | | | TAT GAC AAG GTG GGC CCT AA | 36 | 20 | bp | | | TAC AA | | | | T | C |
| 6 | 19.1 | S0252 | CGC CAC AGA ACG GAC AAA AG | 37 | 20 | 804 | 5 | 163 | CGA TTG GCA GAT AAA | 22 | 89 | C/T | Nipponbare | Others |
| | | | GAC CAA TCC TTT GCC GAA GC | 38 | 20 | bp | | | GTT GGA T | | | | T | C |
| 7 | 35.7 | S0109 | CCG ATG GCA GCA CAA ATC TT | 39 | 20 | 850 | 6 | 624 | TGG CTA GAA GTA GAT | 20 | 90 | C/T | Nipponbare | Others |
| | | | TCA GTT TGG CTT GGG TGT CC | 40 | 20 | bp | | | GCT GC | | | | T | C |
| 7 | 84.1 | S0115 | CCA TTG GTT GGT GTG GCT GT | 41 | 20 | 784 | 7 | 534 | AAA CAG GTG AGG GAA | 20 | 91 | G/T | Nipponbare | Others |
| | | | TGG TCG CGG CTG ATA AGC TA | 42 | 20 | bp | | | AGA TG | | | | T | G |
| 7 | 91.7 | S0107 | TGC GAT GGA GGG AGT ATT GG | 43 | 20 | 808 | 8 | 358 | GAC TGA AAA GTT GTG | 20 | 92 | A/G | Nipponbare | Others |
| | | | TGC GAG CGT ACA CCG CTA GT | 44 | 20 | bp | | | TGT GT | | | | A | G |
| 7 | 99.3 | S0126 | GCT TGA GGC ACG TCA AAA TG | 45 | 20 | 791 | 9 | 475 | CAT GAA ATT ATT ACA | 25 | 93 | G/T | Nipponbare | Others |
| | | | TTC CGT CGT TCA TGT TGG TC | 46 | 20 | bp | | | GAA CTA CAG A | | | | T | G |
| 7 | 105.7 | S0124 | CCC ACG GAA ACA GCC AAA AG | 47 | 20 | 956 | 10 | 323 | AGC ACC TCC CCC TCC | 20 | 94 | A/G | Nipponbare | Others |
| | | | TGC TGC CAT GCA AAG AAT CG | 48 | 20 | bp | | | TCT AA | | | | G | A |
| 8 | 20.2 | S0146 | ATT CGA ACG GGG GAT CCA GT | 49 | 20 | 859 | 11 | 612 | GGA ACT AGC CCG TGA | 20 | 95 | A/C | Nipponbare | Others |
| | | | AGC GGA TCC TGC TGA TGA GG | 50 | 20 | bp | | | CGC TC | | | | C | A |
| 8 | 44.6 | S0135 | GTG CTG CAA AGG GGA GTA TG | 51 | 20 | 852 | 12 | 765 | GAG AGT CGA GAT GAT | 20 | 96 | G/T | Nipponbare | Others |
| | | | CGC CAA CCT CGT AAA TCA AA | 52 | 20 | bp | | | CCA AA | | | | G | T |
| 9 | 55.9 | S0155 | GAA CCT GAG GAC CAA GTG AAA GAG T | 53 | 25 | 1300 | 13 | 571 | CAG CTA TAG CCT AGC | 20 | 97 | G/T | Nipponbare | Others |
| | | | GTA GAG AGG AGA GGG AGA AGG AGG A | 54 | 25 | bp | | | TTG GA | | | | G | T |
| 10 | 5.5 | S0161 | ATA CCA CAG GTG CTG CGT GA | 55 | 20 | 340 | 14 | 660 | GAA GAC AGC TTC TGC | 23 | 98 | A/G | Nipponbare | Others |
| | | | TGC GCA ACT AGG GAT TTT CC | 56 | 20 | bp | | | TGG TTT GT | | | | A | G |

TABLE 9

| | | | | | | | Polymorphism | | | | | | | |
|---|---|---|---|---|---|---|---|---|---|---|---|---|---|---|
| | | Genomic | | | | | (SNP) | | | | | | | |
| | | PCR | | | | | detection | | | | | | | |
| | | | | | | | | | Primer | | | | | |
| | | | | SEQ | | | SEQ | | nucleotide | | SEQ | | | |
| | | Marker | Primer nucleotide | ID | | | ID | Posi- | sequence | | ID | Termi- | | |
| chr | cM | name | sequence (5′-3′) | NO: | mer | Size | NO: | tion | (5′-3′) | mer | NO: | nator | SNPs | |
| 11 | 20.3 | S0177 | CCT TGT GGT CAC ACT TGC GG | 57 | 20 | 488 | 15 | 223 | AAC GTC ATG GAC GAT | 20 | 99 | A/G | Nipponbare | Others |
| | | | CGG TCT TGA GGT CCA GGG TG | 58 | 20 | bp | | | CCG CT | | | | G | A |
| 11 | 35.6 | S0178 | TGG CAT CTT TGC ATG TTG AGC | 59 | 21 | 460 | 16 | 247 | GCC ATG AAA GCA CTG | 20 | 100 | C/T | Nipponbare | Others |
| | | | GCA TCC AGC TGC ACA TTT CC | 60 | 20 | bp | | | AAA AA | | | | C | T |
| 11 | 80.5 | S0174 | GAA TCG GTT GCA GGA GAG GG | 61 | 20 | 311 | 17 | 163 | TTG AGT TCT TGG GGA | 20 | 101 | A/G | Nipponbare | Others |
| | | | GCG GCT ATG CCA TGT TTT TAC C | 62 | 22 | bp | | | TTT GT | | | | G | A |
| 11 | 85.7 | S0185 | CGA CCC CAT GAA GCT TTT GC | 63 | 20 | 644 | 18 | 421 | TGT TAC AAG CAA AGC | 25 | 102 | A/C | Nipponbare | Others |
| | | | AAA TCC ACG ACC TCC ACC CCT | 64 | 21 | bp | | | ATG AGG AAT G | | | | A | C |
| 12 | 42.7 | S0208 | CTC CCT CCG CTC CCA GAA AT | 65 | 20 | 500 | 19 | 178 | AGC TCG AGC TCG AAG | 20 | 103 | C/G | Nipponbare | Others |
| | | | ATT TTG GTG GAG CGT CCC CT | 66 | 20 | bp | | | ATG GC | | | | C | G |
| 1 | 181.8 | S0007 | GCA TGG ATG ACC CTG CTA AT | 67 | 20 | 802 | 20 | 141 | CAA ACA TTT AAA ATA | 28 | 104 | A/G | Nipponbare | Others |
| | | | TGA TGC CGT TGA CTT TTT GA | 68 | 20 | bp | | | TAA ATC ATG AAT A | | | | A | G |
| 5 | 55.5 | S0070 | CTT GCT TGG GCA ATC GTC AA | 69 | 20 | 897 | 21 | 480 | TAA GCC CCC GGC CGA | 25 | 105 | A/G | Nipponbare | Others |
| | | | GTT GCT GAC GCG ACC AGT GT | 70 | 20 | bp | | | ACC GGC AAA G | | | | A | G |
| 8 | 40.2 | S0310 | GCT TTG CTT GTT TGA CCA CTC G | 71 | 22 | 802 | 22 | 481 | GAC TAC AAT CTT CCA | 20 | 106 | C/T | Nipponbare | Others |
| | | | CCA TTT TCA TGT CGT GGC TTG | 72 | 21 | bp | | | CTC CA | | | | T | C |
| 4 | 97.7 | S0375 | ACA CAA GTG TGC CAT TTT GC | 73 | 20 | 901 | 23 | 131 | TGT GAA CTA CAC TAT | 26 | 107 | G/C | Nipponbare | Others |
| | | | TGC CAA GCT ACC TGA GAA CA | 74 | 20 | bp | | | TAA GTT GCT TA | | | | G | C |
| 11 | 35.6 | S0346 | CGT GCT TGG ATT TTT GTA AGC | 75 | 21 | 677 | 24 | 510 | CTG GGA CTT GGA ATG | 21 | 108 | G/A | Nipponbare | Others |
| | | | GCA TCC AGC TGC ACA TTT CC | 76 | 20 | bp | | | TTT GTT | | | | G | A |
| 1 | 161.5 | S0013 | AAA TTC GGA ATG GCT AGC TG | 77 | 20 | 798 | 25 | 248 | GCT AAT GTG AAT TAG | 22 | 109 | C/T | Nipponbare | Others |
| | | | ACC TCC GAT GAT TCA ACC AA | 78 | 20 | bp | | | CCC CCC T | | | | C | T |
| 11 | 55.1 | S0347 | CAA GCG AAG ACT GGA GAG GTT | 79 | 21 | 292 | 26 | 92 | AGT TTA ACT ATA TAT | 28 | 110 | G/C | Nipponbare | Others |
| | | | ACG TGC TGG CCT CCT ATG TT | 80 | 20 | bp | | | AGC ATA CTG ATT C | | | | C | G |
| 3 | 94.9 | S0330 | ATC AAG CAC GAT CGG AAA CG | 81 | 20 | 888 | 27 | 743 | CAT CTT ATG GTT TAG | 23 | 111 | A/G | Nipponbare | Others |
| | | | ATG GCC GTG GAC TCC AAG TT | 82 | 20 | bp | | | GAG GAA TT | | | | A | G |
| 8 | 55.4 | S0336 | GAC GAA ATT GTT TCG CCC CTA | 83 | 21 | 787 | 28 | 552 | GTC TAT TTG GTA CCA | 21 | 112 | C/T | Nipponbare | Others |
| | | | GCC TTC GAG TGG TTT GAC GA | 84 | 20 | bp | | | CTT TCT | | | | C | T |

Furthermore, in another method, DNAs are first prepared from a test rice (step (a)). Then, a DNA comprising a nucleotide described in any of the above (1) to (28), or a nucleotide composing a base pair with the above nucleotide in the complementary strand, is amplified (step (b)). Then, a primer that is complementary to a sequence covering up to the nucleotide sequence next to the nucleotide described in any of the above (1) to (28), or the nucleotide composing a base pair with the above nucleotide in the complementary strand, is synthesized (step (c)). Then (step (d)), a single nucleotide elongation reaction is carried out in the presence of fluorescently labeled nucleotides, using the DNA amplified in step (b) as a template, with the primer synthesized in step (c). Then (step (e)), the nucleotides used in the reaction of step (d) are determined using a sequencer. The nucleotide sequence determined in step (e) is then compared with a control (step (f)). An example of such methods is the SNuPe method (Rapid Commun. Mass Spectrom. 14: 950-959 (2000)).

Furthermore, in another method, DNAs are first prepared from a test rice (step (a)). Then, a DNA comprising a nucleotide described in any of the above (1) to (28), or a nucleotide composing a base pair with the above nucleotide in the complementary strand, is amplified (step (b)). The molecular weight of the DNA amplified in step (b) is then measured using a mass spectrometer (step (c)). The molecular weight obtained in step (c) is then compared with that of a control (step (d)). An example of such a method is the MALDI-TOF MS method (Trends Biotechnol. 18: 77-84 (2000)).

Furthermore, in another method, DNAs are first prepared from a test rice (step (a)). Then, a DNA comprising a nucleotide described in any of the above (1) to (28), or a nucleotide composing a base pair with the above nucleotide in the complementary strand, is amplified (step (b)). Then, a substratum on which a nucleotide probe is immobilized is provided (step (c)).

Herein, “substratum” means a planar material that can immobilize nucleotides. In this invention, nucleotides include oligonucleotides and polynucleotides. The substratum of this invention is not limited to any specific substratum, as long as it allows nucleotide immobilization, but in general, substrata used in DNA array technology may be suitably used. DNA arrays are generally composed of thousands of nucleotides printed on a substratum at high density. Normally, DNAs are printed on the non-porous surface of a substratum. Generally, the surface of a substratum is glass, but a porous membrane such as a nitrocellulose membrane may be used.

In this invention, an example of a method for immobilizing nucleotides (array) is an array developed by Affymetrix, mainly composed of oligonucleotides. In such an oligonucleotide array, the oligonucleotides are normally synthesized in situ. For example, methods for in situ oligonucleotide synthesis using photolithographic technology (Affymetrix) and inkjet for immobilizing chemical compounds (Rosetta Inpharmatics) are already known, and any technique can be used to construct the substrata of this invention.

The nucleotide probes immobilized on the substratum are not limited to any specific probes, as long as they allow detection of single nucleotide polymorphisms at a nucleotide described in any of the above (1) to (28), or a nucleotide composing a base pair with the above nucleotide in the complementary strand. Thus, the probe may be a probe that can specifically hybridize with a DNA comprising a nucleotide described in any of the above (1) to (28), or a nucleotide composing a base pair with the above nucleotide in the complementary strand, for example. The nucleotide probe may not be completely complementary to a DNA comprising the nucleotide described in any of the above (1) to (28), or a nucleotide composing a base pair with the above nucleotide in the complementary strand, as long as the hybridization is specific. In this invention, when an oligonucleotide is immobilized, the length of the nucleotide probe immobilized on a substratum is normally 10 to 100 bases, preferably 10 to 50 bases, and more preferably 15 to 25 bases.

In this method, the DNA of step (b) is then contacted with the substratum of step (c) (step (d)). This step allows the DNA to hybridize with the above nucleotide probe. The solutions and conditions for hybridization may vary depending on many factors, including the length of the nucleotide probe immobilized on the substratum, but hybridization can generally be performed using a method commonly known to one skilled in the art.

In this method, the strength of hybridization between the DNA and nucleotide probe immobilized on the substratum is then detected (step (e)). The detection may be performed by scanning the fluorescence signal on a scanner, for example. In a DNA array, a DNA immobilized on slide glass is generally called a probe, and a labeled DNA in solution is called a target. Thus, in the present description, the above nucleotides immobilized on the substratum are described as nucleotide probes. In this method, the intensity detected in step (e) is then compared with that of a control (step (f)).

Examples of such methods are methods using DNA arrays (Strategies in SNP gene polymorphism. Kennichi Matsubara and Yoshiyuki Sakaki. Nakayama-Shoten p 128-135; Nature Genetics 22: 164-167 (1999)).

In addition to the above methods, allele-specific oligonucleotide (ASO) hybridization may be used to detect only those mutations at specific positions. An oligonucleotide comprising a nucleotide sequence in which a mutation is supposed to exist is prepared, and used for hybridization with a DNA. If a mutation is present, the efficiency of hybrid formation is reduced. Such changes can be detected by methods such as Southern blotting, or methods using the properties of special fluorescent reagents that are quenched upon intercalation into gaps within hybrids, or the like.

In addition, the present invention provides oligonucleotides that are primers for distinguishing between rice varieties, for use in amplifying DNA regions that comprise a nucleotide described in any of the above (1) to (28), or a nucleotide composing a base pair with an above nucleotide in the complementary strand. An example of such an oligonucleotide may be an oligonucleotide designed such that it spans a nucleotide described in any of the above (1) to (28), or a nucleotide composing a base pair with the above nucleotide in the complementary strand. PCR primers may be designed and synthesized by methods commonly known to those skilled in the art. The PCR primers are not limited in length, and are normally 15 to 100 bp, and preferably 17 to 30 bp. The present invention also provides oligonucleotides that comprise a nucleotide sequence complementary to a sequence covering up to a nucleotide next to a nucleotide described in any of the above (1) to (28), or a nucleotide composing a base pair with the above nucleotide in the complementary strand. Such an oligonucleotide is useful as a primer for use in the methods of this invention, for distinguishing rice varieties by the AcycloPrime method, for example. Examples of such oligonucleotides are those shown in Table 8 or 9.

Furthermore, the present invention provides oligonucleotides comprising at least 15 nucleotides, for use in the methods for distinguishing between rice varieties, which can hybridize with a DNA region comprising a nucleotide described in any of the above (1) to (28), or a nucleotide composing a base pair with the above nucleotide in the complementary strand. The oligonucleotides can be used as a probe, for example.

Such oligonucleotides can hybridize specifically with a DNA region comprising a nucleotide described in any of the above (1) to (28), or a nucleotide composing a base pair with the above nucleotide in the complementary strand. Here, to “hybridize specifically” means that the oligonucleotide does not generate a significant amount of cross-hybridization with other DNAs under standard hybridization conditions, and preferably under stringent conditions (for example, the conditions described in Sambrook et al. Molecular Cloning. Cold Spring Harbor Laboratory Press, New York, U.S.A. second edition, (1989)). As long as hybridization is specific, an oligonucleotide does not need to be completely complementary to a DNA region comprising a nucleotide as described in any of the above (1) to (28), or a nucleotide composing a base pair with the above nucleotide in the complementary strand. The length of the oligonucleotide is not limited, as long as it is 15 nucleotides or longer. Such oligonucleotides can be prepared using a commercial oligonucleotide synthesizer, for example. Alternatively, they may be prepared as double-stranded DNA fragments obtained by restriction enzyme treatment and the like.

In addition, the oligonucleotides to be used are preferably appropriately labeled. The methods for labeling may include a method in which the 5′-end of the oligonucleotide is labeled with ³²P by phosphorylating with T4 polynucleotide kinase, a method in which nucleotide substrates labeled with an isotope such as ³²P, a fluorescent dye, biotin, or the like are incorporated into the oligonucleotides using a primer such as random hexamer oligonucleotides and a DNA polymerase such as Klenow enzyme (the random primer method). Furthermore, the present invention also includes oligonucleotides of 15 nucleotides or longer that comprise a polymorphic mutation, according to any of the above (1′) to (28′), in a nucleotide described in any of the above (1) to (28), or a nucleotide composing a base pair with the above nucleotide in the complementary strand.

Furthermore, the present invention provides kits for distinguishing rice varieties, comprising the above oligonucleotides of the invention. The kits of this invention may further comprise an alkaline aqueous solution. The kits may be also packaged with a standard rice sample for use as a control, instructions describing the method for using the kit, and the like.

### BRIEF DESCRIPTION OF THE DRAWINGS

FIG. 1 shows the nucleotide sequence of SEQ ID NO: 1, which indicates a site polymorphic between 24 rice varieties, as well as the sequences of primers for amplifying a DNA region comprising that site.

FIG. 2 shows the nucleotide sequence of SEQ ID NO: 2, which indicates a site polymorphic between 24 rice varieties, as well as the sequences of primers for amplifying a DNA region comprising that site.

FIG. 3 shows the nucleotide sequence of SEQ ID NO: 3, which indicates a site polymorphic between 24 rice varieties, as well as the sequences of primers for amplifying a DNA region comprising that site.

FIG. 4 shows the nucleotide sequence of SEQ ID NO: 4, which indicates a site polymorphic between 24 rice varieties, as well as the sequences of primers for amplifying a DNA region comprising that site.

FIG. 5 shows the nucleotide sequence of SEQ ID NO: 5, which indicates a site polymorphic between 24 rice varieties, as well as the sequences of primers for amplifying a DNA region comprising that site.

FIG. 6 shows the nucleotide sequence of SEQ ID NO: 6, which indicates a site polymorphic between 24 rice varieties, as well as the sequences of primers for amplifying a DNA region comprising that site.

FIG. 7 shows the nucleotide sequence of SEQ ID NO: 7, which indicates a site polymorphic between 24 rice varieties, as well as the sequences of primers for amplifying a DNA region comprising that site.

FIG. 8 shows the nucleotide sequence of SEQ ID NO: 8, which indicates a site polymorphic between 24 rice varieties, as well as the sequences of primers for amplifying a DNA region comprising that site.

FIG. 9 shows the nucleotide sequence of SEQ ID NO: 9, which indicates a site polymorphic between 24 rice varieties, as well as the sequences of primers for amplifying a DNA region comprising that site.

FIG. 10 shows the nucleotide sequence of SEQ ID NO: 10, which indicates a site polymorphic between 24 rice varieties, as well as the sequences of primers for amplifying a DNA region comprising that site.

FIG. 11 shows the nucleotide sequence of SEQ ID NO: 11, which indicates a site polymorphic between 24 rice varieties, as well as the sequences of primers for amplifying a DNA region comprising that site.

FIG. 12 shows the nucleotide sequence of SEQ ID NO: 12, which indicates a site polymorphic between 24 rice varieties, as well as the sequences of primers for amplifying a DNA region comprising that site.

FIG. 13 shows the nucleotide sequence of SEQ ID NO: 13, which indicates a site polymorphic between 24 rice varieties, as well as the sequences of primers for amplifying a DNA region comprising that site.

FIG. 14 shows the nucleotide sequence of SEQ ID NO: 14, which indicates a site polymorphic between 24 rice varieties, as well as the sequences of primers for amplifying a DNA region comprising that site.

FIG. 15 shows the nucleotide sequence of SEQ ID NO: 15, which indicates a site polymorphic between 24 rice varieties, as well as the sequences of primers for amplifying a DNA region comprising that site.

FIG. 16 shows the nucleotide sequence of SEQ ID NO: 16, which indicates a site polymorphic between 24 rice varieties, as well as the sequences of primers for amplifying a DNA region comprising that site.

FIG. 17 shows the nucleotide sequence of SEQ ID NO: 17, which indicates a site polymorphic between 24 rice varieties, as well as the sequences of primers for amplifying a DNA region comprising that site.

FIG. 18 shows the nucleotide sequence of SEQ ID NO: 18, which indicates a site polymorphic between 24 rice varieties, as well as the sequences of primers for amplifying a DNA region comprising that site.

FIG. 19 shows the nucleotide sequence of SEQ ID NO: 19, which indicates a site polymorphic between 24 rice varieties, as well as the sequences of primers for amplifying a DNA region comprising that site.

FIG. 20 shows the nucleotide sequence of SEQ ID NO: 20, which indicates a site polymorphic between 24 rice varieties, as well as the sequences of primers for amplifying a DNA region comprising that site.

FIG. 21 shows the nucleotide sequence of SEQ ID NO: 21, which indicates a site polymorphic between 24 rice varieties, as well as the sequences of primers for amplifying a DNA region comprising that site.

FIG. 22 shows the nucleotide sequence of SEQ ID NO: 22, which indicates a site polymorphic between 24 rice varieties, as well as the sequences of primers for amplifying a DNA region comprising that site.

FIG. 23 shows the nucleotide sequence of SEQ ID NO: 23, which indicates a site polymorphic between 24 rice varieties, as well as the sequences of primers for amplifying a DNA region comprising that site.

FIG. 24 shows the nucleotide sequence of SEQ ID NO: 24, which indicates a site polymorphic between 24 rice varieties, as well as the sequences of primers for amplifying a DNA region comprising that site.

FIG. 25 shows the nucleotide sequence of SEQ ID NO: 25, which indicates a site polymorphic between 24 rice varieties, as well as the sequences of primers for amplifying a DNA region comprising that site.

FIG. 26 shows the nucleotide sequence of SEQ ID NO: 26, which indicates a site polymorphic between 24 rice varieties, as well as the sequences of primers for amplifying a DNA region comprising that site.

FIG. 27 shows the nucleotide sequence of SEQ ID NO: 27, which indicates a site polymorphic between 24 rice varieties, as well as the sequences of primers for amplifying a DNA region comprising that site.

FIG. 28 shows the nucleotide sequence of SEQ ID NO: 28, which indicates a site polymorphic between 24 rice varieties, as well as the sequences of primers for amplifying a DNA region comprising that site.

FIG. 29 shows the nucleotide sequence of SEQ ID NO: 29, which indicates a site polymorphic between 24 rice varieties, as well as the sequences of primers for amplifying a DNA region comprising that site.

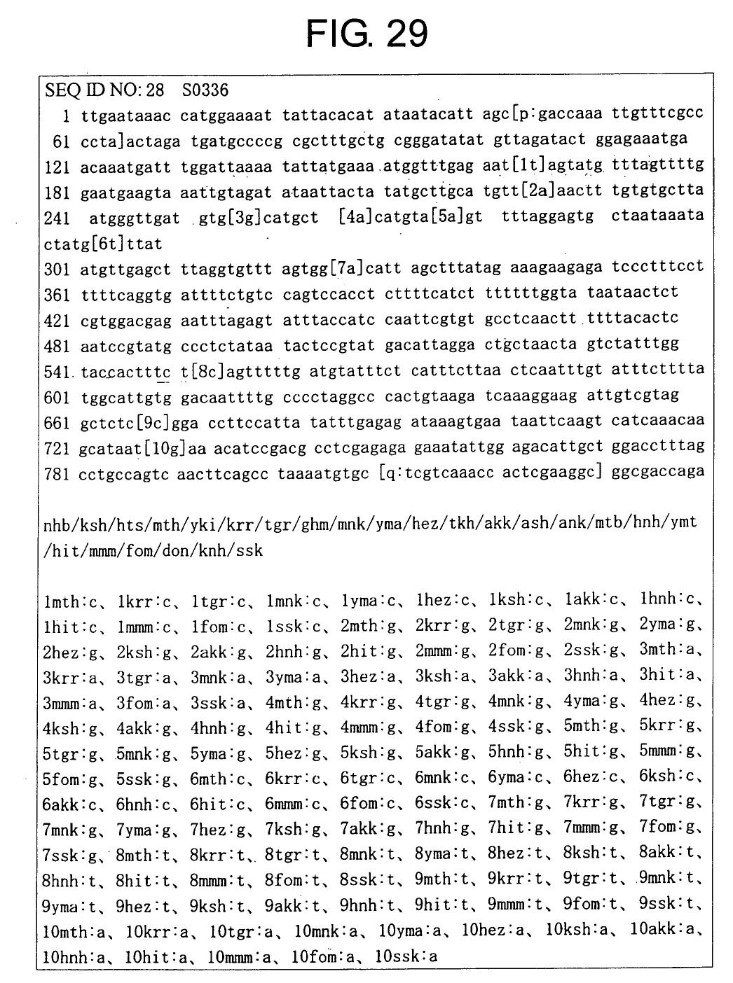

FIG. 30 shows a photograph showing the results of PCR using DNA extracted from polished rice as a template. The polished rice sample was a commercial rice, said to be “Akitakomachi produced in Ibaraki Prefecture in Heisei 12 (the year 2000)”. The primers used for the PCR were PGC1001 (U: 5′-accgggtagggaaacaaaac-3′/SEQ ID NO: 113; L: 5′-aataatacttcggcgcatcg-3′/SEQ ID NO: 114). PCR was carried out using DNA extracted by the methods below as a template, and the reaction mixture was separated by electrophoresis on a 1.5% agarose gel.

M: molecular weight marker (φX/HaeIII);

1: Method 1 (CTAB);

2: Method 2 (alkali+CTAB);

3: Method 3 (simple extraction);

4: Method 4 (simple extraction+phenol:chloroform treatment);

5: Method 5 (alkali+simple extraction);

6: Method 6 (alkali+simple extraction+phenol:chloroform treatment);

7: control (DNA extracted from a green leaf of Habataki by CTAB, 40 ng); and

8: control (DNA extracted from a green leaf of Sasanishiki by CTAB, 40 ng).

### BEST MODE FOR CARRYING OUT THE INVENTION

This invention will be explained in detail below with reference to Examples, but it is not to be construed as being limited thereto.

### EXAMPLE 1

### Detection of Single Nucleotide Polymorphisms (SNPs)

Primers for amplifying 800 bp to 1 kbp of rice genomic DNA were designed using publicly available rice genome analysis information on the Rice Genome Research Program homepage (http://rgp.dna.affrc.go.jp/), and rice genomic sequences registered in DDBJ (http://www.ddbj.nig.ac.jp/). Regions not predicted to comprise genes were mainly used for the chromosomal regions with publicly available rice genomic nucleotide sequences, and RFLP marker probe sequences and the like were used for regions other than these. The primer design support site, Primer3 (http://www-genome.wi.mit.edu/cgi-bin/primer/primer3_www.cgi), was used to design the primers.

Using the designed primers, first, PCR amplification was performed using Ampli Taq Gold (Applied Biosystems) and DNA extracted by a simple method from rice varieties Nipponbare, Koshihikari, Kasalath, Guang-lu-ai 4 (G4, below), Kitaake, and a wild rice (Oryza rufipogon, W1943) as a template. To confirm the amplified fragments, a portion of the reaction mixture was separated by electrophoresis on an agarose gel. The rest of the reaction mixture was treated with ExoSAP-IT (Amersham Biosciences) to remove unreacted primers and dNTPs, and then subjected to a sequencing reaction as a template. One of the original primers, used for the first amplification, was again added to the template, and samples for sequencing were prepared by performing a cycle sequencing reaction using the DYEnamic ET Dye Terminator Cycle Sequencing kit for MegaBACE (Amersham Biosciences). Sequencing was carried out using the MegaBACE 1000 DNA Sequencing System (Molecular Dynamics). The obtained sequence data was compared between the varieties to search for single nucleotide substitution polymorphisms. Sequencing was performed at least twice for each variety with each primer, and only certain cases were considered to be polymorphisms.

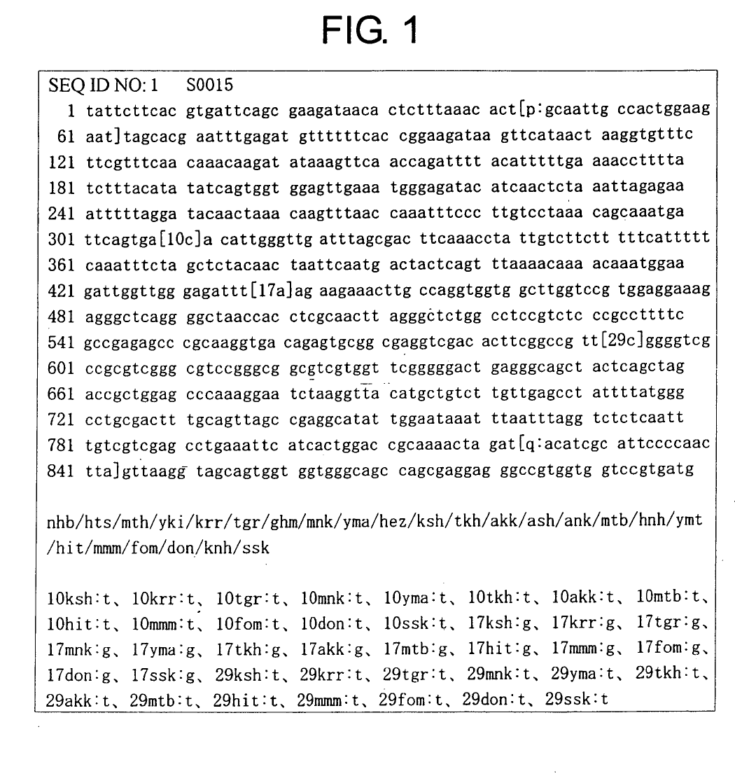

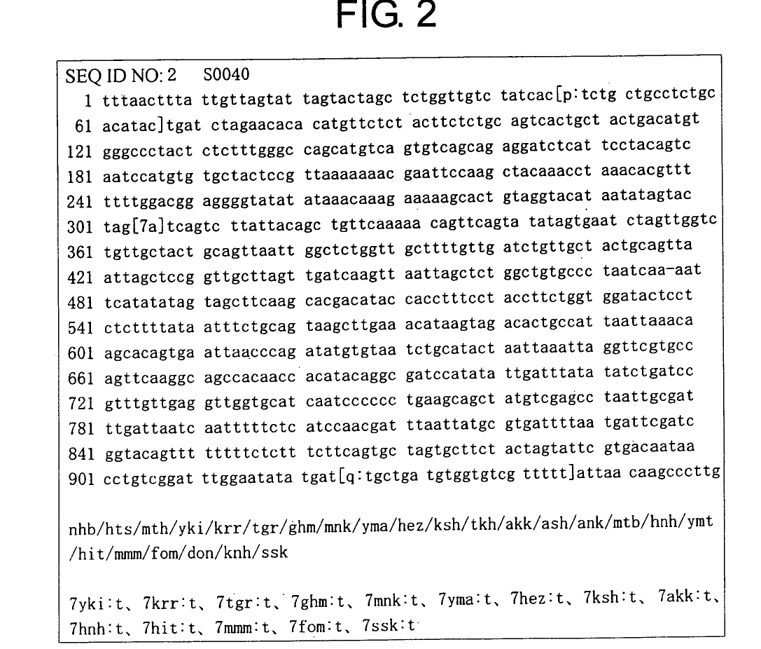

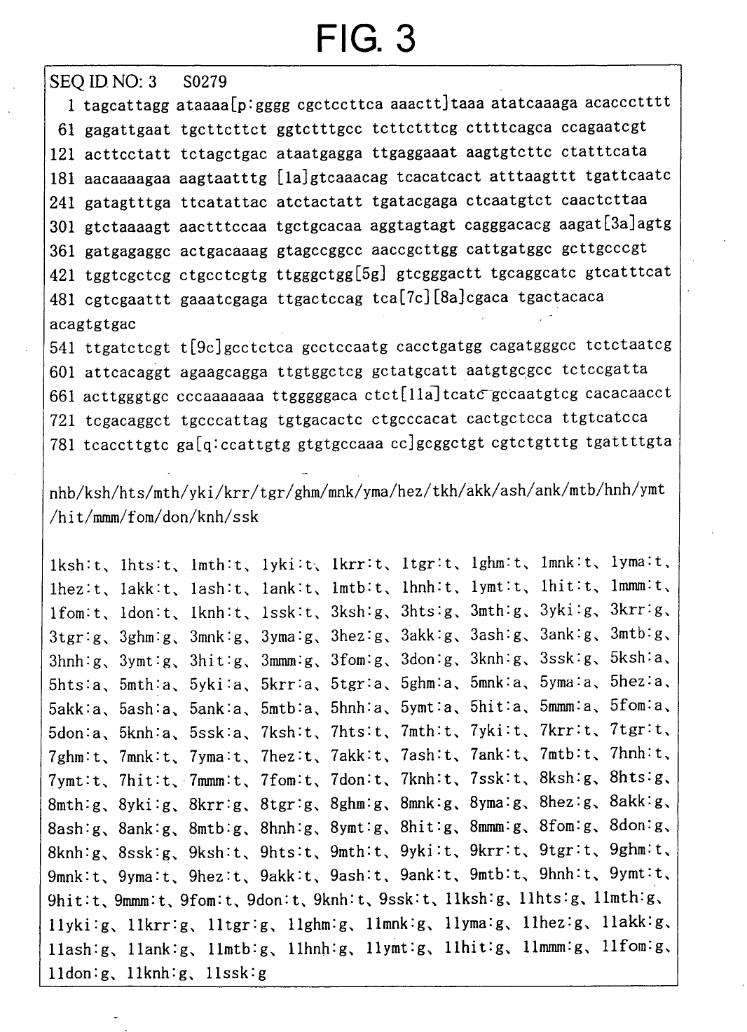

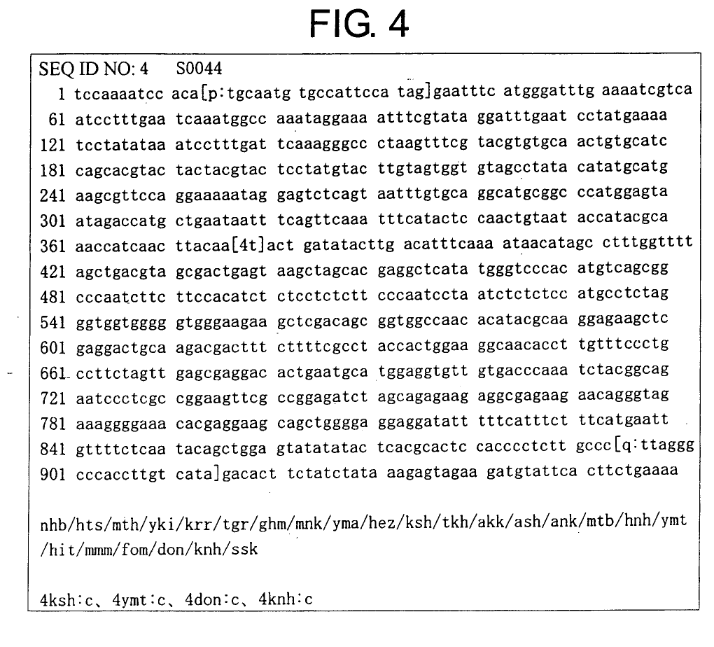

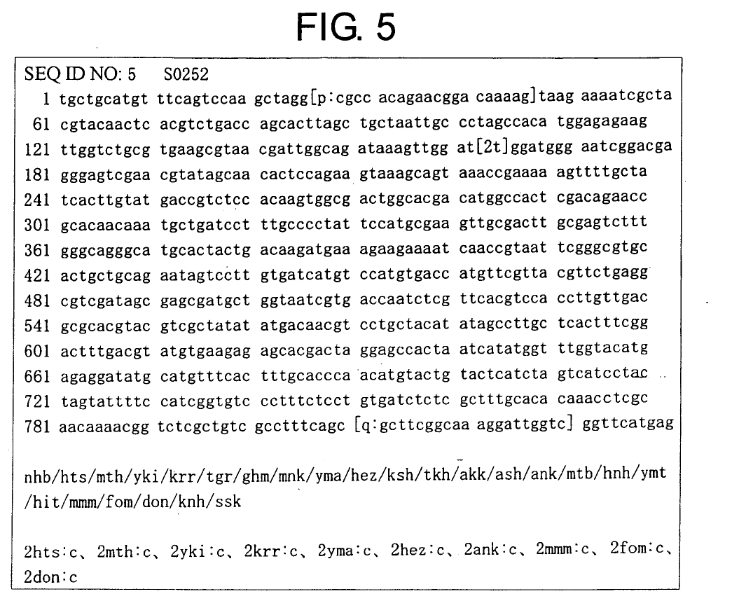

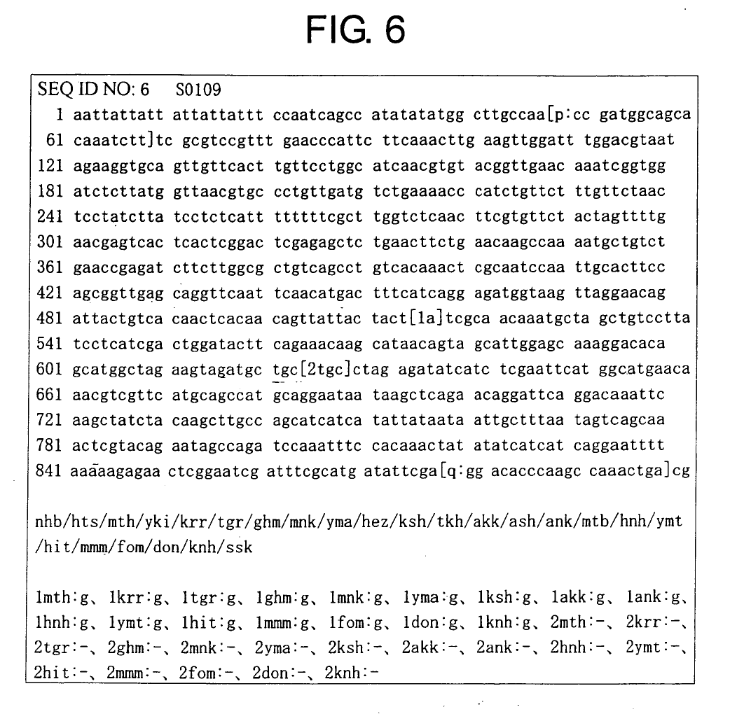

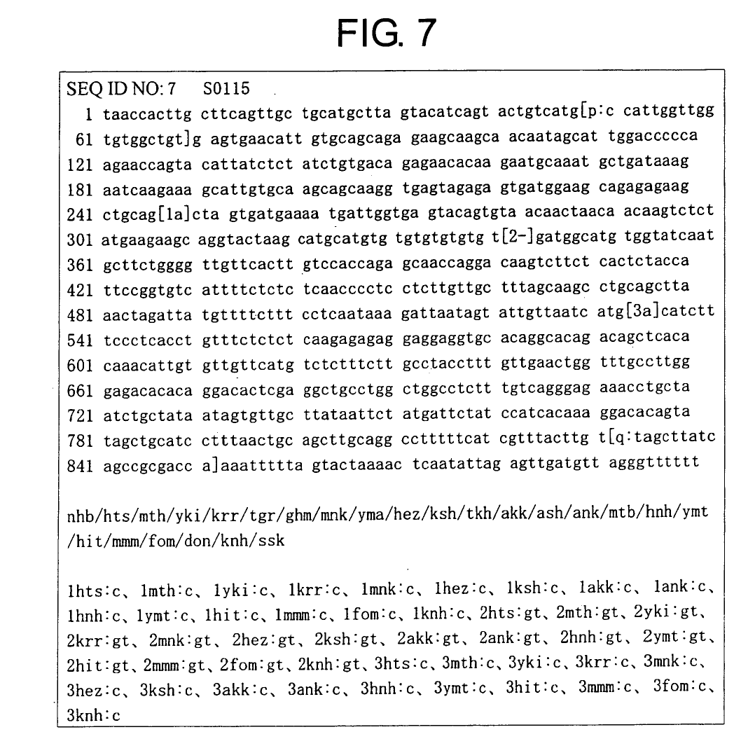

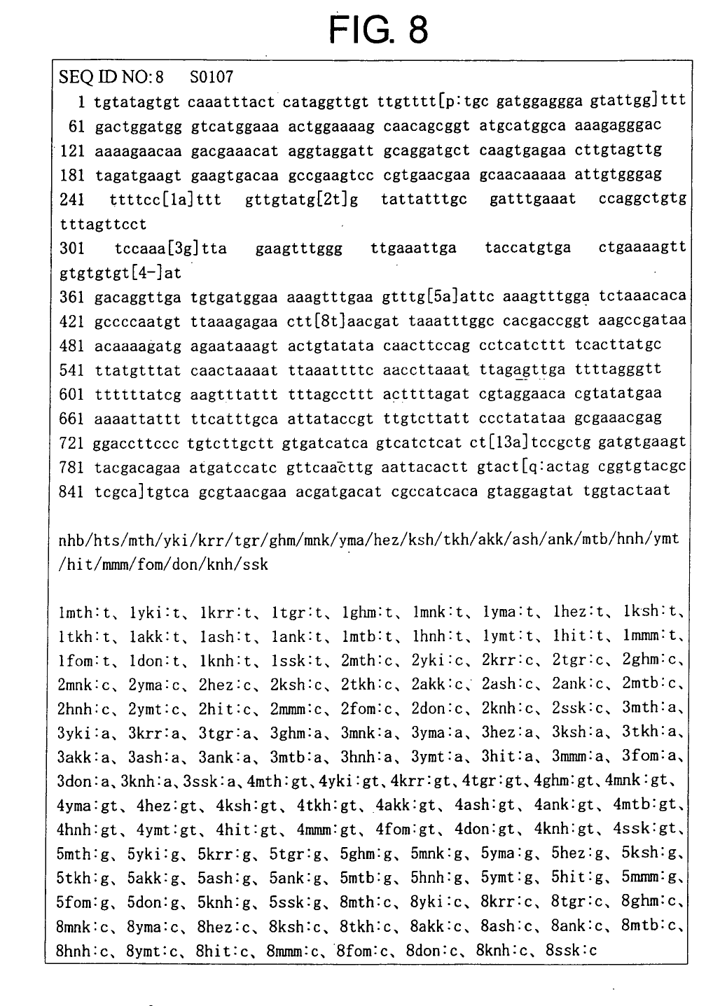

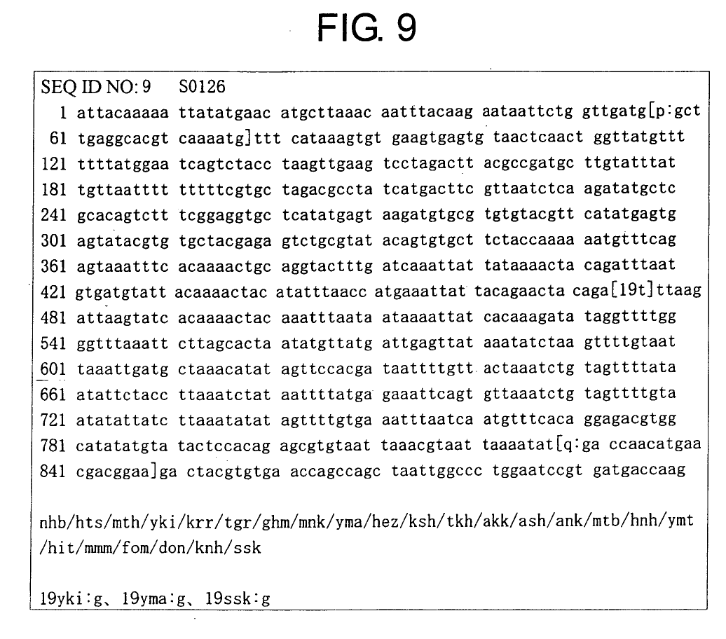

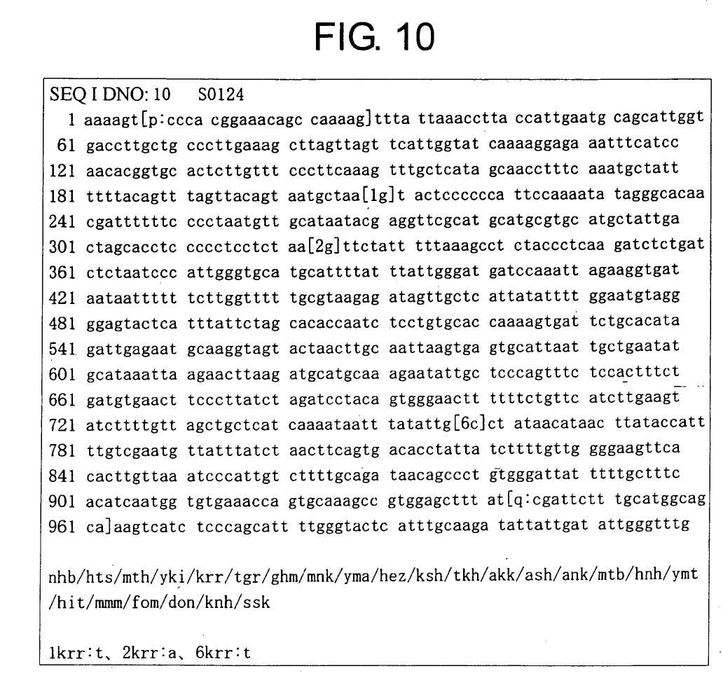

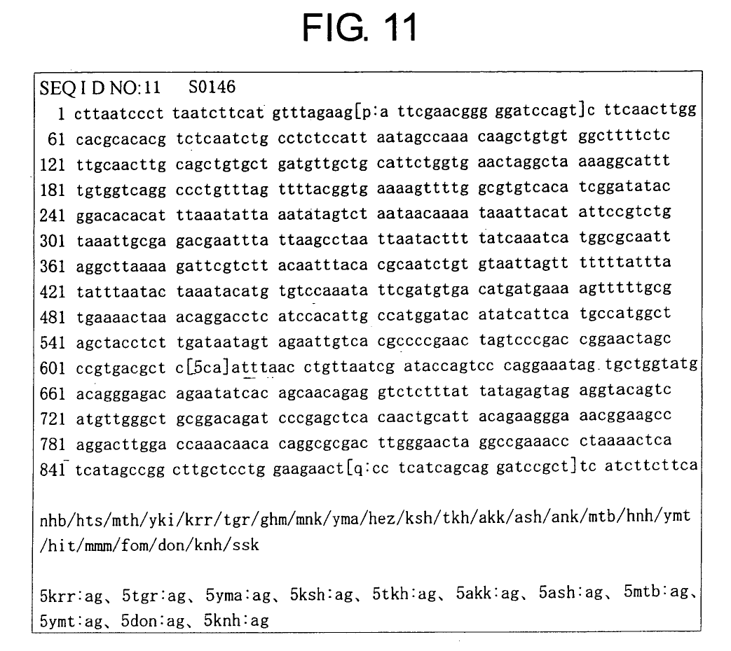

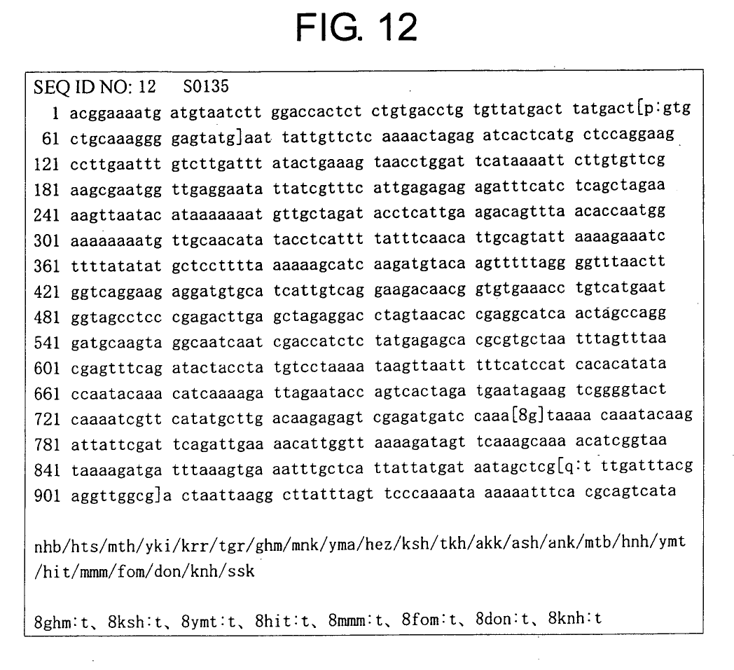

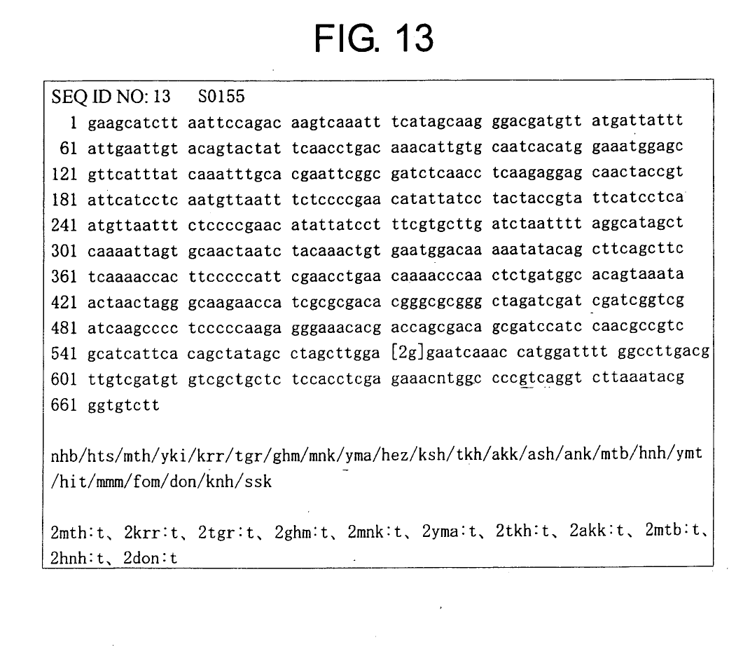

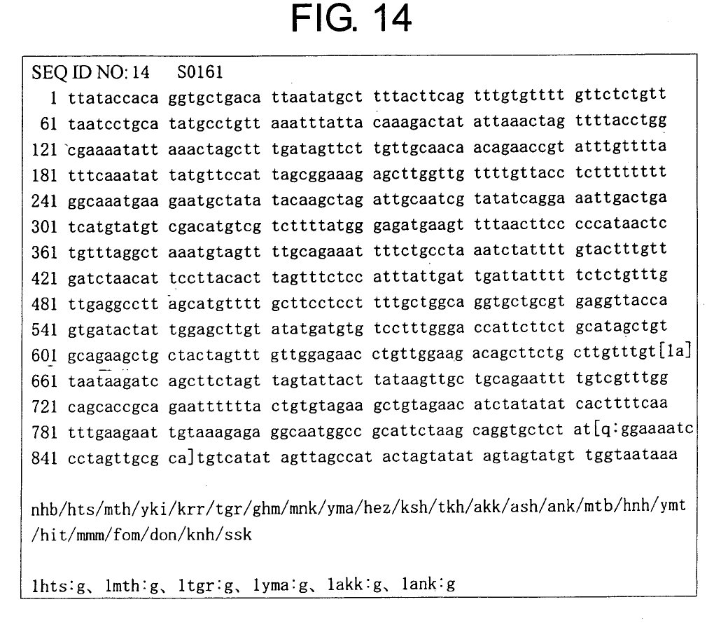

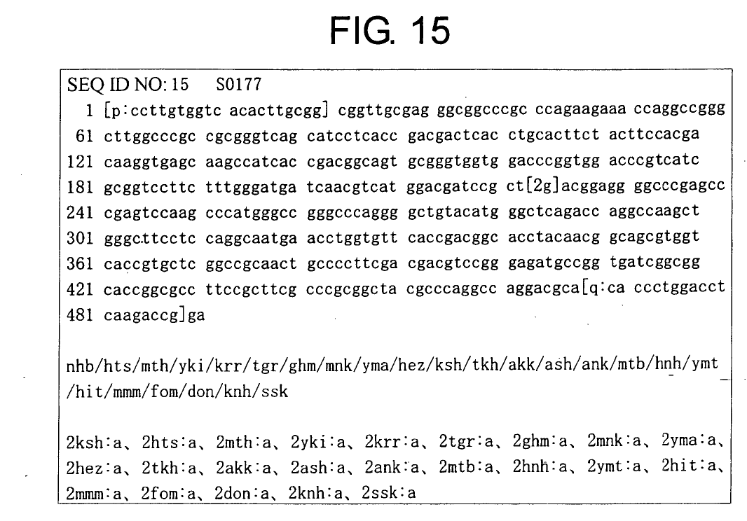

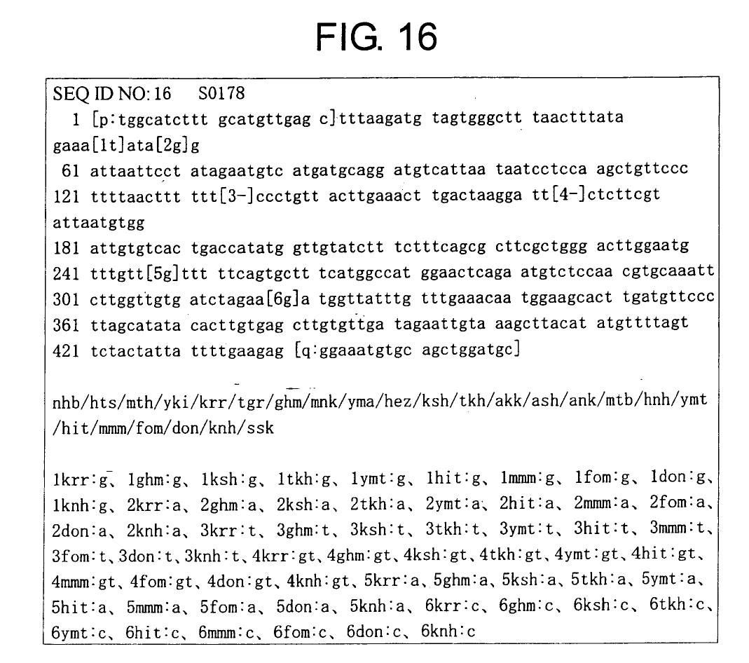

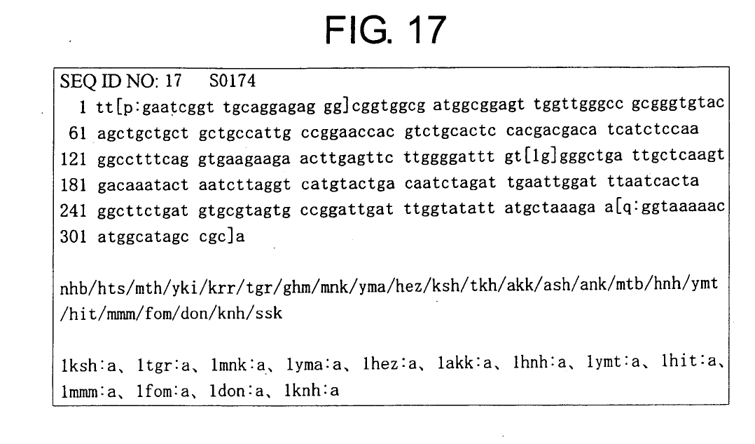

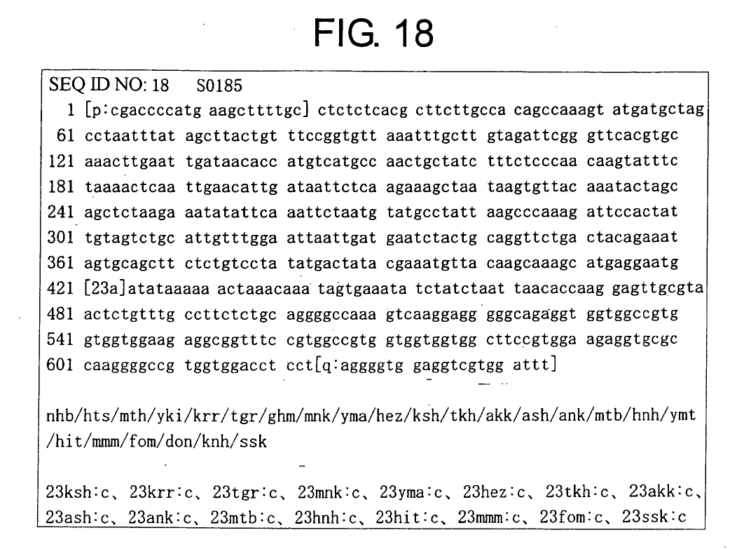

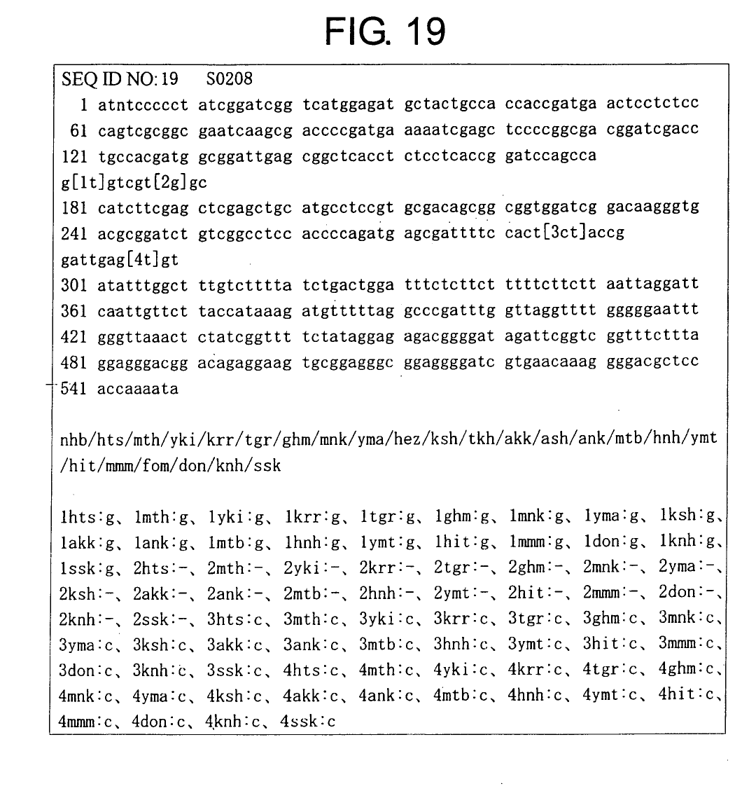

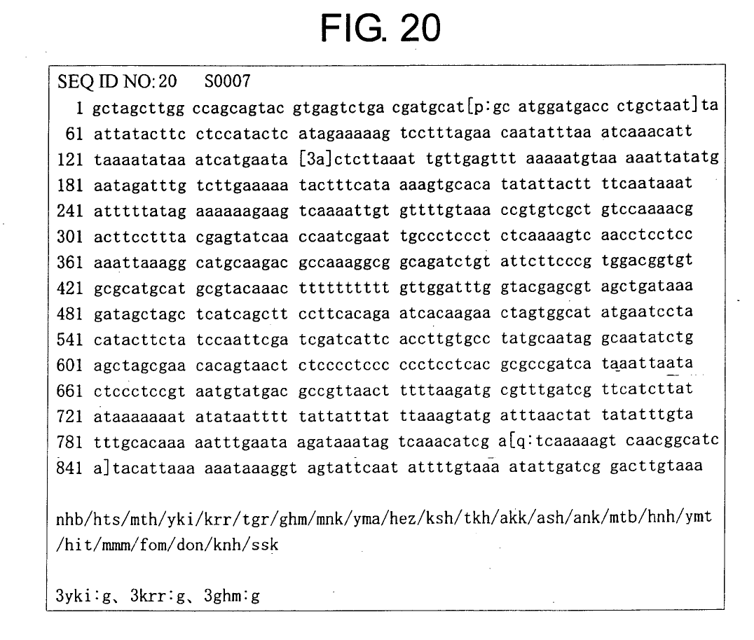

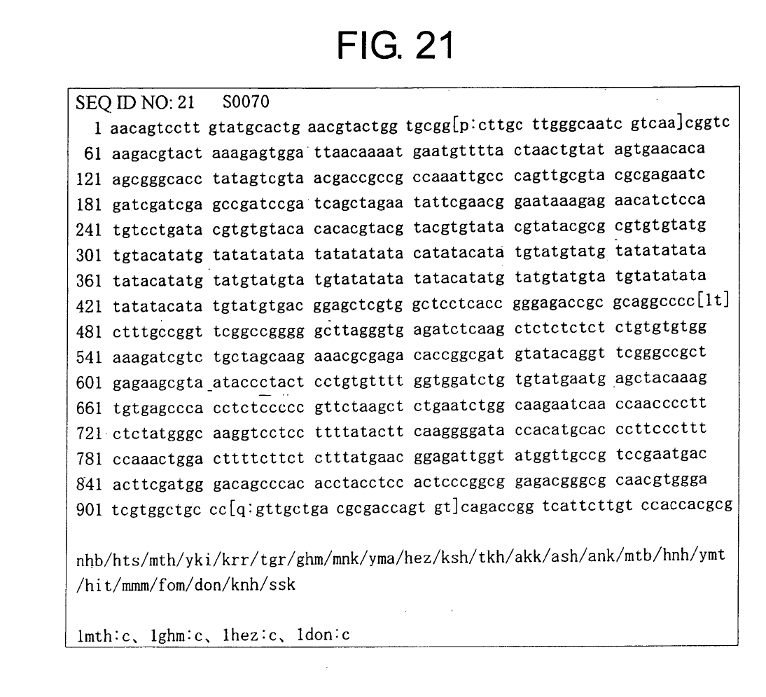

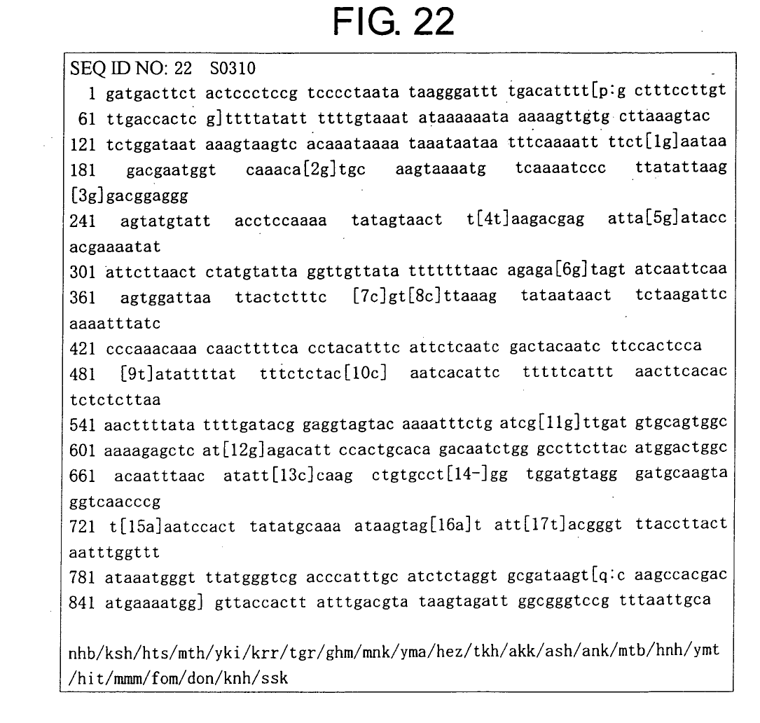

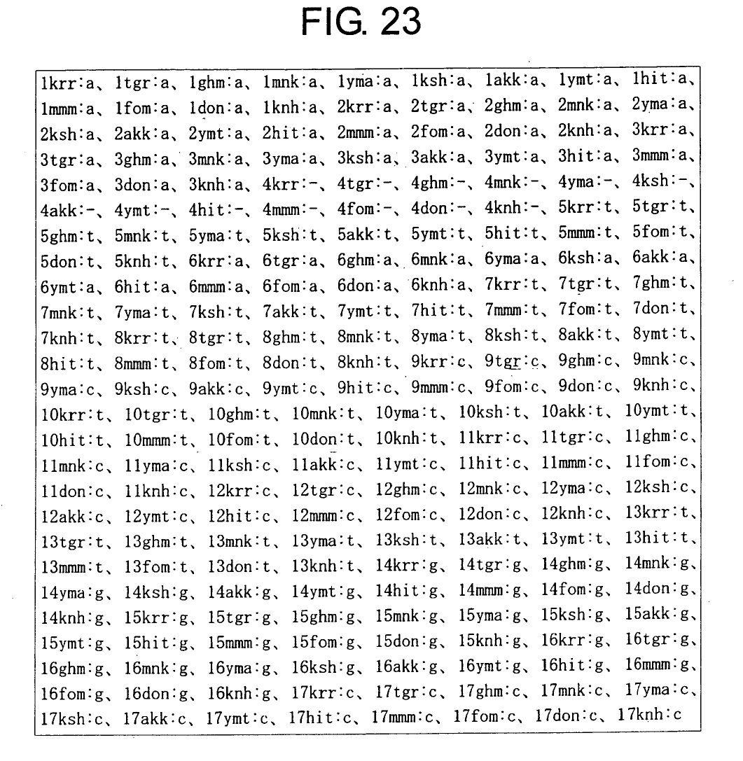

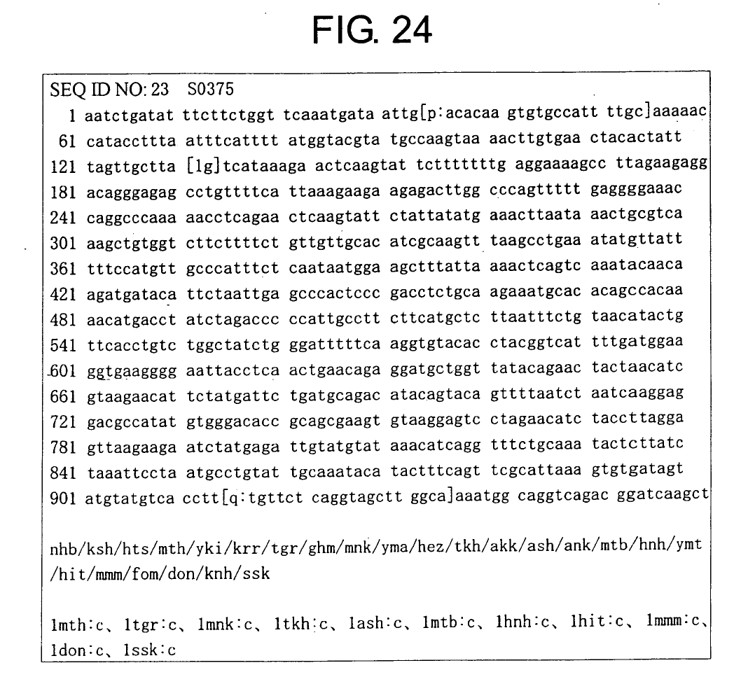

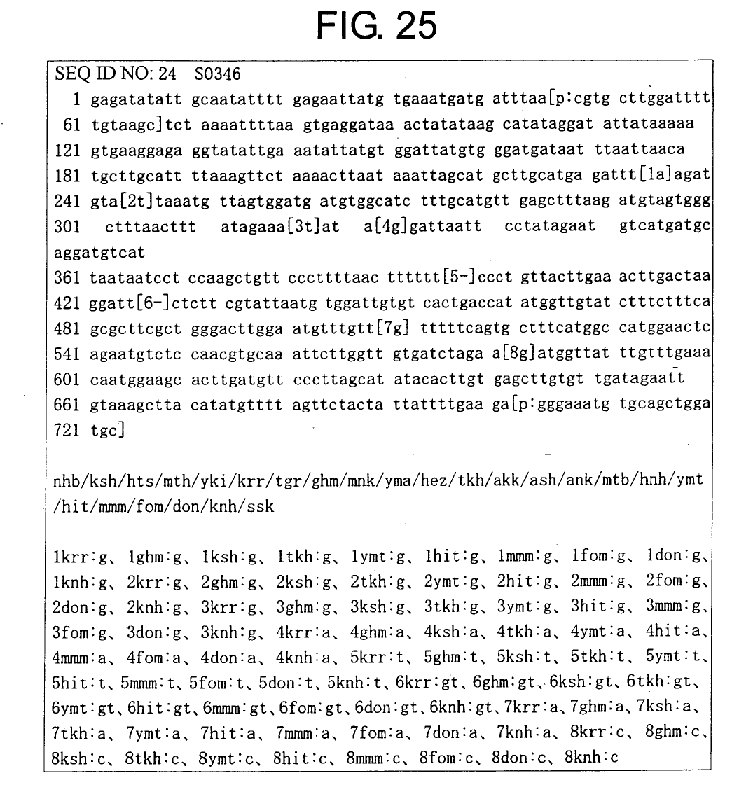

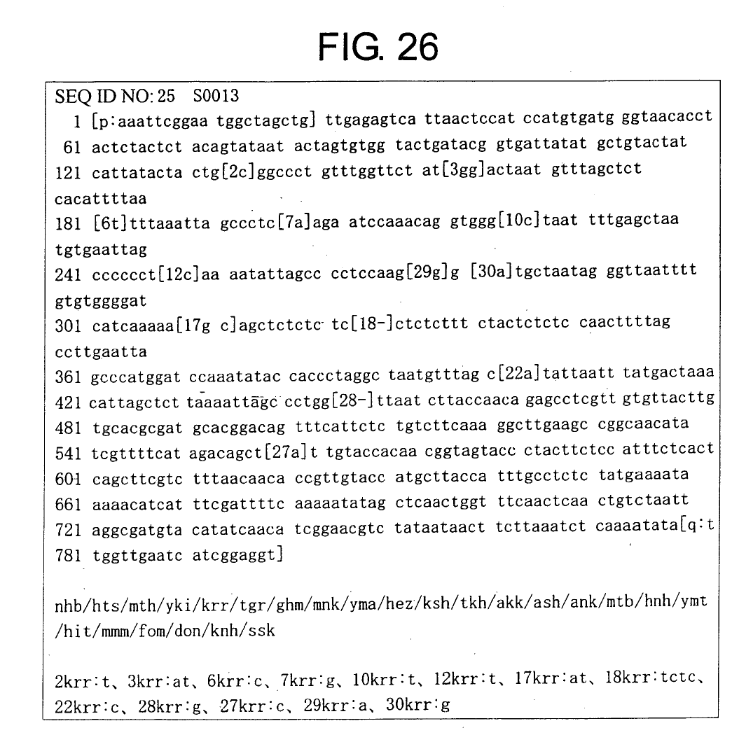

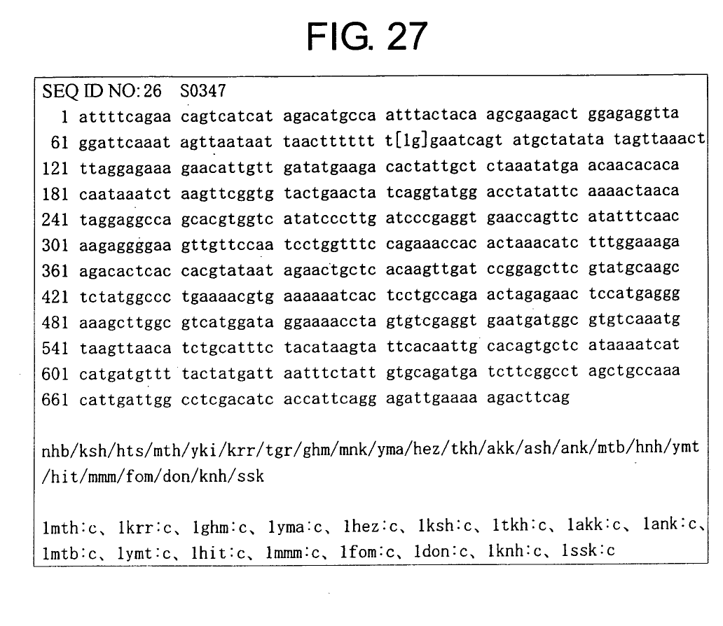

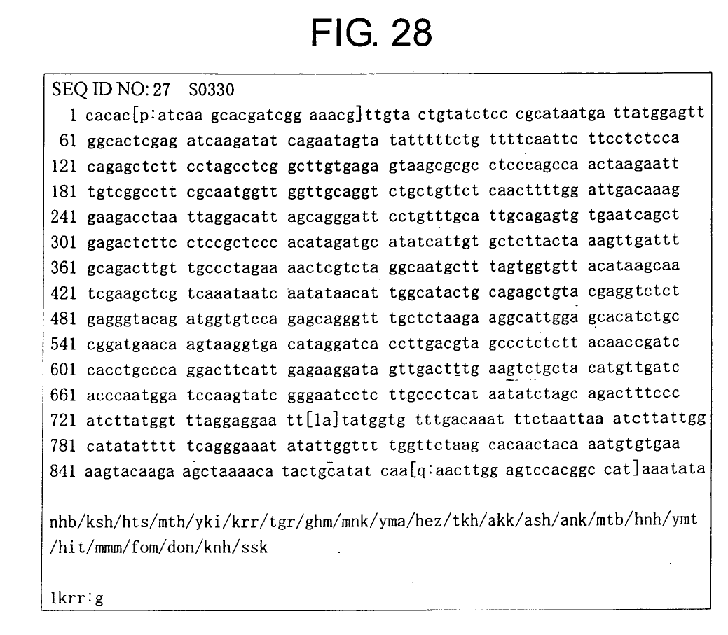

Sites showing single nucleotide polymorphisms between Nipponbare and Koshihikari, and between Nipponbare and Kitaake, were further examined by similarly performing PCR and sequencing using genomic DNA extracted by a simple method from Nipponbare, Hatsushimo, Mutsuhomare, Yukinosei, Kirara 397, Tsugaruroman, Gohyakumangoku, Morinokumasan, Yumeakari, hanaechizen, Koshihikari, Tsukinohikari, Akitakomachi, Asanohikari, Aichinokaori, Matsuribare, Hinohikari, Yumetsukushi, Hitomebore, Manamusume, Fusaotome, Dontokoi, Kinuhikari, and Sasanishiki as templates, and comparing the nucleotide sequences at the polymorphic sites for each of the varieties. FIGS. 1 to 28 show the polymorphisms found among the above 24 rice varieties. Polymorphic data are shown according to the following rules:

[Rules for Data Description]

(1) Primer sites are indicated by brackets, and the upper primer site and lower primer site are marked with “p:” and “q:”, respectively.

Example: actctactta a[p:gcagagcga tgaacctgca] atattgagaa aactc [q:aatcacgccc atccttgcct]

(2) SNP positions are shown by brackets and an identification number.

Example: cg[1a]agag[2aa]cttc[3a[4c4]cattt gggg[5c5]acac3]c

Note: In general, identification numbers were attached to both the beginning and ending brackets; however, the number might be omitted from the latter bracket where the correspondence was obvious.

(3) The analyzed variety is indicated by a code below the attached sequence. Variety codes are separated by “/”.

Example: nhb/ksh/kal/gla/pwl/kta

[Variety code] Each of the above rice varieties is indicated by an abbreviation using three alphabet letters. For example, Nipponbare and Koshihikari are “nhb” and “ksh”, respectively.

(4) The variety code was followed by SNP data, shown as “identification number, variety code: SNP”.

Example: 1 ksh:g

### OTHER EXAMPLES

(5) Deletions are indicated by “−”. Regardless of the number of deleted nucleotides, only one “−” was used.

Example: g[5agg]ggtcat ctgttacatt atag

5kal:−

(6) Where deletions occurred in the same position but varied in length depending on the variety:

Example: gtttg[20a:gtat[20b:t ccattatgta ttatttcatt tgct20b]t20a]ttatg

20akal:−, 20bgla:−

Since the deletion occurs in the same position, the same identification number was used. However, differences in deletion length were clarified by alphabet letters to differentiate, such as “20a:” and “20b:”.

(7) For insertions, “−” was inserted in the published sequence. A single “−” was used.

Example: tacaca[7−]gtca attttattca

7kal:aa

Next, primers for detecting SNPs were designed for those SNPs useful in distinguishing the varieties, and a single nucleotide terminator reaction was performed using the AcycloPrime-FP kit (Perkin Elmer), to prepare samples for genotyping. Genotyping was performed by measuring fluorescence polarization with ARVO (Perkin Elmer).

The results showed that the markers generated at those positions determined by sequencing to be SNPs showed distinct patterns among the varieties, and could be used in combination for variety classification (Tables 2 to 7). Tables 8 and 9 show data for the generated SNP markers, such as primer sequences and the SNP sites used.

### EXAMPLE 2

### Examination of the Methods for DNA Extraction From Polished Rice, Unpolished Rice, and Cooked Rice

Methods for extracting DNA from polished, unpolished, and cooked rice were examined. First, a single kernel of polished, unpolished, and cooked rice was placed into a 2 ml tube (Eppendorf), and 0.4 ml of extraction buffer (1 M KCl, 10 mM Tris-HCl, 1 mM EDTA, 0.1 N NaOH) and zirconia balls of 3 mm in diameter were added thereto. The tubes were stood with their lid on for 30 minutes at 4° C. The kernels were disrupted using a Retch disrupter mixer mill MM300 for two rounds at 300 Hz for two minutes, and a milky solution was obtained. The solution was centrifuged at 10,000 rpm for ten minutes, and the resulting supernatant (0.3 ml) was transferred to a fresh tube. After the addition of 0.3 ml of isopropanol, the solution was well mixed and centrifuged again at 10,000 rpm for ten minutes. The supernatant was discarded, and 1 ml of 70% ethanol was added to the pellet, and then centrifuged at 10,000 rpm for three minutes. The supernatant was discarded, and the pellet was dried and then dissolved in 30 μl of sterilized water (Method 5).

Alternatively, after transferring the supernatant (0.3 ml) to a fresh tube in Method 5, 0.3 ml of phenol:chloroform (1:1) was added, and the solution was mixed well and centrifuged at 10,000 rpm for ten minutes. The supernatant was then transferred to a fresh tube, and processed for isopropanol precipitation (Method 6).

Alternatively, the composition of the extraction buffer used in Methods 5 and 6 was changed to 1 M KCl, 10 mM Tris-HCl, and 1 mM EDTA (Methods 3 and 4, respectively).

In other alternative methods, CTAB extraction was used. Specifically, a polished rice kernel and 0.2 ml of CTAB buffer (Method 1), or 0.2 ml of 0.1 N NaOH (Method 2) were put in a 2 ml tube, and zirconia balls of 3 mm in diameter were added thereto. With the tube top closed, the kernel was disrupted under the same conditions as for Method 5. 0.7 ml of CTAB buffer was then added, and heated at 56° C. for 20 minutes. 640 μl of phenol:chloroform (1:1) was added to the solution, which was then mixed, and then centrifuged at 10,000 rpm for ten minutes. The supernatant (0.7 ml) was transferred to a fresh tube, and 1.3 ml of CTAB precipitation buffer was added. This was then centrifuged at 10,000 rpm for ten minutes. The pellet was dissolved by adding 0.5 ml of 1 N NaCl containing RNase, then 1 ml ethanol was added, mixed, and centrifuged at 10,000 rpm for ten minutes. The pellet was washed with 1 ml of 70% ethanol, dried, and dissolved in 30 μl of sterilized water.

The DNAs obtained by the above methods were used as templates for PCR using the primers PGC1001 (U: 5′-accgggtagggaaacaaaac-3′/SEQ ID NO: 113; L: 5′-aataatacttcggcgcatcg-3′/SEQ ID NO: 114).

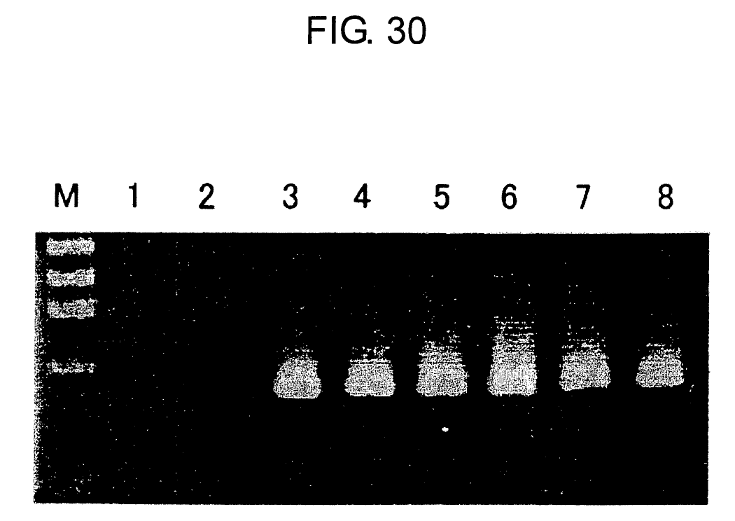

These results are shown in FIG. 30. While no amplified product was obtained using DNA extracted from polished rice by Method 1 or 2, good amplification was observed with DNA extracted using Methods 3 to 6. This accordingly indicates that phenol:chloroform treatment is unnecessary when extracting DNA from polished rice, and thus that Methods 3 or 5 are the simplest methods. The difference between Methods 3 and 5 lay in the buffer used to disrupt the kernels, which was alkaline in Method 5. An alkaline buffer is advantageous in that the polished rice tissues are quickly rendered fragile, and satisfactory disruption is easily achieved. Thus, Method 5 was chosen as the simplest and most efficient method.

For unpolished rice and cooked rice, an amplified product was not obtained for DNA extracted by Methods 1 and 2, although amplification was observed for that of Methods 3 to 6. The best amplification was observed for DNA extracted by Method 6. Thus, a method using alkaline buffer and phenol:chloroform treatment was shown most effective for extracting DNA from unpolished or cooked rice.

### EXAMPLE 3

### Distinguishing Varieties of Polished Rice

Commercial polished rice indicated to be “100% Akitakomachi produced in Ibaraki Prefecture in Heisei 12 (the year 2000)” was purchased. 32 kernels were randomly selected, and DNA was separately extracted from every single kernel using Method 5. PCR was carried out using the extracted DNAs as templates, and primers for the three markers (S0115, S0146, and S0178) necessary and sufficient to distinguish Akitakomachi from the other 25 rice varieties. Furthermore, AcycloPrime reactions were performed using the PCR products as templates, and the single nucleotide polymorphisms were determined.

As a result, 27 kernels were identified as Akitakomachi, but three kernels turned out to be varieties other than Akitakomachi. Two of these kernels were not distinguished since one of the three markers did not give a result. Based on their patterns, the three kernels determined not to be Akitakomachi were presumably either Kirara 397, Koshihikari, Yumetsukushi, or Kinuhikari.

The above results confirmed that the present invention could be used to distinguish between varieties of polished rice.

### EXAMPLE 4

### Identification of Varieties of Polished Rice

In order to determine the variety of the three kernels which were determined in Example 3 to not be Akitakomachi, and which might be Kirara 397, Koshihikari, Yumetsukushi, or Kinuhikari, PCR was performed using the extracted DNAs as templates and primers for the two markers (S0015, S0045) required and sufficient to distinguish between the three varieties. Furthermore, AcycloPrime reactions were performed using the PCR products as templates, and the single nucleotide polymorphisms were determined.

The results showed that all three of the kernels had the same pattern as Koshihikari. Therefore, it was presumed that the polished rice used in Example 3 very likely contained Koshihikari in addition to Akitakomachi.

### EXAMPLE 5

### Inspection of the Blending Ratio of Polished Rice

A polished rice said to be “Kirara 397, 30%; Tsugaruroman, 40%; Hitomebore, 30%” was inspected to determine whether the three varieties were blended as indicated. 32 kernels were randomly selected from the polished rice, and DNA was separately extracted from every kernel using Method 5. PCR was performed using the extracted DNAs as templates, and primers for the seven markers (S0115, S0135, S0161, S0252, S0310, S0336, and S0375) necessary and sufficient to distinguish Kirara 397, Tsugaruroman, and Hitomebore from among the 26 rice varieties that can be distinguished. Furthermore, AcycloPrime reactions were performed using the PCR products as templates, and the single nucleotide polymorphisms were determined.

The results indicated that seven kernels were from Kirara 397, eleven were from Tsugaruroman, and five were from Hitomebore, while two kernels were not from any of these three varieties. The other seven kernels were not determined since some of the seven markers did not provide data. According to the ratio of the three varieties based on the 25 kernels for which data could be collected, the blending ratio of the inspected polished rice was presumed to be Kirara 397, 28%; Tsugaruroman, 44%; and Hitomebore, 20%; with other varieties being 4%.

### INDUSTRIAL APPLICABILITY

The present invention provides methods for distinguishing between rice varieties. Traditional methods for distinguishing varieties based on their cultivation traits require inspection by the eyes of experienced breeders, and thus simple distinctions are difficult. Furthermore, the variety of every rice kernel could not be distinguished. In contrast, the methods of this invention examine polymorphisms in the rice genome, and thus enable varieties to be accurately distinguished using a minute amount of rice sample. Furthermore, the methods of this invention can be applied to accurately distinguish between closely related varieties.

1. A method of distinguishing between rice varieties, comprising the following steps (a) and (b):
(a) determining the type of a nucleotide at a position according to any of the following (1) to (28) in the rice genome, or a nucleotide on the complementary strand that composes a base pair with the nucleotide at the position:
(1) position 593 in the nucleotide sequence of SEQ ID NO: 1,
(2) position 304 in the nucleotide sequence of SEQ ID NO: 2,
(3) position 450 in the nucleotide sequence of SEQ ID NO: 3,
(4) position 377 in the nucleotide sequence of SEQ ID NO: 4,
(5) position 163 in the nucleotide sequence of SEQ ID NO: 5,
(6) position 164 in the nucleotide sequence of SEQ ID NO: 6,
(7) position 534 in the nucleotide sequence of SEQ ID NO: 7,
(8) position 358 in the nucleotide sequence of SEQ ID NO: 8,
(9) position 475 in the nucleotide sequence of SEQ ID NO: 9,
(10) position 323 in the nucleotide sequence of SEQ ID NO: 10,
(11) position 612 in the nucleotide sequence of SEQ ID NO: 11,
(12) position 765 in the nucleotide sequence of SEQ ID NO: 12,
(13) position 571 in the nucleotide sequence of SEQ ID NO: 13,
(14) position 660 in the nucleotide sequence of SEQ ID NO: 14,
(15) position 223 in the nucleotide sequence of SEQ ID NO: 15,
(16) position 247 in the nucleotide sequence of SEQ ID NO: 16,
(17) position 163 in the nucleotide sequence of SEQ ID NO: 17,
(18) position 421 in the nucleotide sequence of SEQ ID NO: 18,
(19) position 178 in the nucleotide sequence of SEQ ID NO: 19,
(20) position 141 in the nucleotide sequence of SEQ ID NO: 20,
(22) position 480 in the nucleotide sequence of SEQ ID NO: 21,
(22) position 481 in the nucleotide sequence of SEQ ID NO: 22,
(23) position 131 in the nucleotide sequence of SEQ ID NO: 23,
(24) position 510 in the nucleotide sequence of SEQ ID NO: 24,
(25) position 248 in the nucleotide sequence of SEQ ID NO: 25,
(26) position 92 in the nucleotide sequence of SEQ ID NO: 26,
(27) position 743 in the nucleotide sequence of SEQ ID NO: 27, and
(28) position 552 in the nucleotide sequence of SEQ ID NO: 28, and
(b) relating the type of the nucleotide determined in step (a) to a variety of rice.

2. The method of claim 1, which distinguishes the type of a nucleotide by using a polymorphic marker characterized by a mutation of any of the following (1) to (28) in the rice genome:
(1) the nucleotide at position 593 in the nucleotide sequence of SEQ ID NO: 1 is T,
(2) the nucleotide at position 304 in the nucleotide sequence of SEQ ID NO: 2 is T,
(3) the nucleotide at position 450 in the nucleotide sequence of SEQ ID NO: 3 is A,
(4) the nucleotide at position 377 in the nucleotide sequence of SEQ ID NO: 4 is C,
(5) the nucleotide at position 163 in the nucleotide sequence of SEQ ID NO: 5 is C,
(6) the nucleotide at position 624 in the nucleotide sequence of SEQ ID NO: 6 is C,
(7) the nucleotide at position 534 in the nucleotide sequence of SEQ ID NO: 7 is C,
(8) the nucleotide at position 358 in the nucleotide sequence of SEQ ID NO: 8 is G,
(9) the nucleotide at position 475 in the nucleotide sequence of SEQ ID NO: 9 is G,
(10) the nucleotide at position 323 in the nucleotide sequence of SEQ ID NO: 10 is A,
(11) the nucleotide at position 612 in the nucleotide sequence of SEQ ID NO: 11 is A,
(12) the nucleotide at position 765 in the nucleotide sequence of SEQ ID NO: 12 is T,
(13) the nucleotide at position 571 in the nucleotide sequence of SEQ ID NO: 13 is T,
(14) the nucleotide at position 660 in the nucleotide sequence of SEQ ID NO: 14 is G,
(15) the nucleotide at position 223 in the nucleotide sequence of SEQ ID NO: 15 is A,
(16) the nucleotide at position 247 in the nucleotide sequence of SEQ ID NO: 16 is A,
(17) the nucleotide at position 163 in the nucleotide sequence of SEQ ID NO: 17 is A,
(18) the nucleotide at position 421 in the nucleotide sequence of SEQ ID NO: 18 is C,
(19) the nucleotide at position 178 in the nucleotide sequence of SEQ ID NO: 19 is G,
(20) the nucleotide at position 141 in the nucleotide sequence of SEQ ID NO: 20 is G,
(21) the nucleotide at position 480 in the nucleotide sequence of SEQ ID NO: 21 is C,
(22) the nucleotide at position 481 in the nucleotide sequence of SEQ ID NO: 22 is C,
(23) the nucleotide at position 131 in the nucleotide sequence of SEQ ID NO: 23 is C,
(24) the nucleotide at position 510 in the nucleotide sequence of SEQ ID NO: 24 is A,
(25) the nucleotide at position 248 in the nucleotide sequence of SEQ ID NO: 25 is T,
(26) the nucleotide at position 92 in the nucleotide sequence of SEQ ID NO: 26 is C,
(27) the nucleotide at position 743 in the nucleotide sequence of SEQ ID NO: 27 is G, and
(28) the nucleotide at position 552 in the nucleotide sequence of SEQ ID NO: 28 is T.

3. The method of claim 1 or comprising the following steps (a) to (c):
(a) preparing DNA from a test rice,
(b) amplifying a DNA comprising a nucleotide in a position of any of (1) to (28) of claim 1, or a nucleotide in the complementary strand composing a base pair with the nucleotide at the position, and
(c) determining the nucleotide sequence of the amplified DNA.

4. The method of claim 1 I comprising the following steps (a) to (d):
(a) preparing DNA from a test rice,
(b) digesting the prepared DNA with a restriction enzyme,
(c) fractionating the DNA fragments by size, and
(d) comparing the size of the detected DNA fragment with a control.

5. The method of claim 1, comprising the following steps (a) to (e):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) digesting the amplified DNA with a restriction enzyme,
(d) fractionating the DNA fragments by size, and
(e) comparing the size of the detected DNA fragment with a control.

6. The method of claim 1 or comprising the following steps (a) to (e):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) denaturing the amplified DNA into single-stranded DNA,
(d) fractionating the denatured single-stranded DNA on a non-denaturing gel, and
(e) comparing the mobility of the fractionated single-stranded DNA on the gel with a control.

7. The method of claim 1 o, comprising the following steps (a) to (f):
(a) preparing DNA from a test rice,
(b) synthesizing two different oligonucleotide probes labeled with a reporter fluorescence dye and quencher fluorescence dye, where an oligonucleotide is complementary to a proximal nucleotide sequence comprising a nucleotide in a position of any of (1) to (28) of claim 1, or a nucleotide in the complementary strand composing a base pair with the nucleotide at the position,
(c) hybridizing the DNA prepared in step (a) with the probe synthesized in step (b),
(d) amplifying a DNA comprising a nucleotide in a position of any of (1) to (28) of claim 1, or a nucleotide in the complementary strand composing a base pair with the nucleotide at the position,
(e) detecting the emission of reporter fluorescence, and
(f) comparing the emission of reporter fluorescence detected in step (e) with a control.

8. The method of claim 1 I; comprising the following steps (a) to (h):
(a) preparing DNA from a test rice,
(b) synthesizing a probe in which a sequence complementary to the 3′-flanking nucleotide sequence comprising a nucleotide in a position of any of (1) to (28) of claim 1, or a nucleotide in the complementary strand composing a base pair with the nucleotide at the position, is combined with a totally unrelated sequence,
(c) synthesizing a probe that is complementary to the 5′-flanking region comprising a nucleotide in a position of any of (1) to (28) of claim 1, or a nucleotide in the complementary strand composing a base pair with the nucleotide at the position,
(d) hybridizing the probe synthesized in step (c) with the DNA prepared in step (a),
(e) digesting the hybridized DNA in step (d) with a single-stranded DNA cleaving enzyme, and dissociating a part of the probe synthesized in step (b),
(f) hybridizing the dissociated probe in step (e) with a probe for detection,
(g) enzymatically digesting the hybridized DNA in step (f), and measuring the fluorescence intensity thus generated, and
(h) comparing the fluorescence intensity measured in step (g) with a control.

9. The method of claim 1 o, comprising the following steps (a) to (f):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) denaturing the amplified DNA into single-stranded DNAs,
(d) separating only one strand from the denatured single-stranded DNAs,
(e) performing an elongation reaction from near a position of any of (1) to (28) of claim 1, or a nucleotide in the complementary strand composing a base pair with the nucleotide at the position, whereby the reaction elongates one nucleotide at a time, then enzymatically illuminating the generated pyrophosphate, and measuring the intensity of the illumination, and
(f) comparing the fluorescence intensity measured in step (e) with a control.

10. The method of claim 1, comprising the following steps (a) to (f):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) synthesizing a probe complementary to a nucleotide sequence comprising a sequence covering up to a nucleotide adjacent to a position of any of (1) to (28) of claim 1, or a nucleotide in the complementary strand composing a base pair with the nucleotide at the position,
(d) performing a single nucleotide extension reaction in the presence of fluorescently labeled nucleotides, using the DNA amplified in step (b) as a template, and the primer synthesized in step (c),
(e) measuring the fluorescence polarization, and
(f) comparing the fluorescence polarization measured in step (e) with a control.

11. The method of claim 1 o, comprising the following steps (a) to (f):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) synthesizing a primer complementary to a nucleotide sequence comprising a sequence covering up to the nucleotide adjacent to a position of any of (1) to (28) of claim 1, or a nucleotide in the complementary strand composing a base pair with the nucleotide at the position,
(d) performing a single nucleotide extension reaction in the presence of fluorescently labeled nucleotides, using the DNA amplified in step (b) as a template, and the primer synthesized in step (c),
(e) determining the nucleotide variety used in the reaction of step (d) using a sequencer, and
(f) comparing the nucleotide determined in step (e) with a control.

12. The method of claim 1 o, comprising the following steps (a) to (d):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) measuring the molecular weight of the DNA amplified in step (b) using a mass spectrometer, and
(d) comparing the molecular weight measured in step (c) with a control.

13. The method of claim 1 o, comprising the following steps (a) to (f):
(a) preparing DNA from a test rice,
(b) amplifying a DNA comprising a nucleotide in a position of any of (1) to (28) of claim 1, or a nucleotide in the complementary strand composing a base pair with the nucleotide at the position,
(c) providing a substratum on which a nucleotide probe is immobilized,
(d) contacting the DNA of step (b) with the substratum of step (c),
(e) detecting the strength of hybridization between the DNA and the nucleotide probe immobilized on the substratum, and
(f) comparing the strength detected in step (e) with a control.

14. The method of claim 1, further comprising the following steps (a) and (b):
(a) disrupting a rice seed in an alkaline aqueous solvent, and
(b) extracting rice genomic DNA from the seed disrupted in step (a).

15. The method of claim 14, wherein the rice seed is polished.

16. A primer for distinguishing between rice varieties, wherein the primer is
(a) an oligonucleotide for amplification of a DNA region comprising a nucleotide in a position of any of (1) to (28) of claim 1 in the rice genome, or a nucleotide in the complementary strand composing a base pair with the nucleotide at the position, or
(b) an oligonucleotide comprising a nucleotide sequence complementary to a sequence covering up to a nucleotide adjacent to a position of any of (1) to (28) of claim 1 in the rice genome, or a nucleotide in the complementary strand composing a base pair with the nucleotide at the position.

17. An oligonucleotide for distinguishing between rice varieties, wherein the oligonucleotide hybridizes with a DNA region comprising a nucleotide in a position of any of (1) to (28) of claim 1, or a nucleotide in the complementary strand composing a base pair with the nucleotide at the position, comprising at least 15 nucleotides.

18. A kit for distinguishing between rice varieties, comprising the oligonucleotide of claim 17.

19. The kit of claim 18, further comprising an alkaline aqueous solvent.

20. A kit for distinguishing between rice varieties, comprising the primer of claim 16.

21. The kit of claim 20, further comprising an alkaline aqueous solvent.
